(19) United States
(12) Patent Application Publication
(10) Pub. No.: US 2015/0250725 A1
(43) Pub. Date: Sep. 10, 2015

(54) METHOD FOR PRODUCING A LIPID PARTICLE, THE LIPID PARTICLE ITSELF AND ITS USE

(71) Applicant: HOFFMANN-LA ROCHE INC., Little Falls, NJ (US)

(72) Inventors: Martin Bader, Penzberg (DE); Monika Baehner, Munich (DE); Adelbert Grossmann, Eglfing (DE); Anton Jochner, Oberammergau (DE); Hubert Kettenberger, Munich (DE); Silke Mohl, Basel (CH)

(73) Assignee: HOFFMANN-LA ROCHE INC., Little Falls, NJ (US)

(21) Appl. No.: 14/704,705

(22) Filed: May 5, 2015

Related U.S. Application Data
(63) Continuation of application No. 13/217,536, filed on Aug. 25, 2011, now abandoned.

(30) Foreign Application Priority Data
Aug. 30, 2010 (EP) 10008994.5
Aug. 30, 2010 (EP) 10008995.2

(51) Int. Cl. A61K 9/127; C07K 14/775; C07K 14/47; A61K 38/17

(52) U.S. Cl. CPC A61K 9/1275; A61K 38/1709; A61K 9/1277; C07K 14/775; C07K 14/47; C07K 2319/00

(57) ABSTRACT

A method for producing a lipid particle comprising the following: i) providing a first solution comprising denatured apolipoprotein, ii) adding the first solution to a second solution comprising at least two lipids and a detergent but no apolipoprotein, and iii) removing the detergent from the solution obtained in ii) and thereby producing a lipid particle.

### CROSS-REFERENCE TO RELATED APPLICATIONS

This application claims priority under 35 U.S.C. §119 to European Application No. EP 10008994.5 filed Aug. 30, 2010 and European Application No. EP 10008995.2 filed Aug. 30, 2010, the disclosures of which are incorporated herein by reference.

### FIELD OF THE INVENTION

The current invention is in the field of lipoproteins and lipid particles. It is reported herein a method for producing a lipid particle comprising an apolipoprotein, a phosphatidylcholine and a lipid, wherein the formation of the lipid particle is performed in the presence of the synthetic detergent Zwittergent.

### BACKGROUND OF THE INVENTION

Plasma lipoproteins are soluble protein-lipid complexes that carry out lipid transport and metabolism in blood. Several major classes of lipoproteins are distinguished on the basis of their density, size, chemical compositions, and functions. Among them high-density-lipoprotein (HDL) particles alternatively denoted as high-density-lipid particles, are made up of several subclasses that vary in their average molecular weight of from 180 kDa to 360 kDa. Their average lipid and protein content is 50% by weight of each. Phosphatidylcholine (PC) accounts for 38% of the total lipid followed by cholesteryl esters and small amounts of other polar and non-polar lipids, including free cholesterol. The main protein component is apolipoprotein A-I (Apo A-I), representing about 60% of total protein weight in human HDL.

Cholesterol in the human body, especially in circulating body fluids such as blood, is not present as isolated molecule but in form of a complex with certain proteins (lipoproteins). The major fraction of the cholesterol is complexed with low density lipoprotein (LDL) or with high density lipoprotein (HDL). LDL particles comprise apolipoprotein B as major proteinaceous compound whereas HDL particles comprise apolipoprotein A-I as major proteinaceous compound.

Cholesterol taken up by HDL particles is esterified by the enzyme lecithin-cholesterol-acyl-transferase (LCAT). The cholesterol ester has an increased hydrophobicity and diffuses towards the core of the HDL particle. The HDL-cholesterol-ester particle may be delivered to the liver and removed from circulation.

HDL particles and its major polypeptide apolipoprotein A-I participate in the reverse cholesterol transport (RCT). Therein the apolipoprotein A-I increases the efflux of cholesterol from cells, e.g. from cells of the wall of blood vessels, the binding of the lipid and the activation of the lecithin-cholesterol-acetyl-transferase and thereby the elimination of cholesterol via plasmatic flow by the liver. This is an active transport process involving the cell membrane protein ATP-binding-cassette-transporter-A-I (ABCA-I).

Apolipoprotein A-I and apolipoprotein-based therapeutics, e.g. reconstituted HDL particles, were already identified in the late 70ties and early 80ties of the last century. For apolipoprotein A-I-Milano containing lipid particles the clinical proof (meaning significant plaque reduction in arteriosclerotic patients) could be shown. Apolipoprotein A-I-Milano, a dimeric form of wild-type apolipoprotein A-I, was designed according to a naturally occurring mutant of the apolipoprotein A-I molecule. The dimer formation is enabled by the exchange of amino acid residue 173 (arginine) by cysteine allowing the formation of a disulfide bond.

In WO 2009/131704 nanostructures suitable for sequestering cholesterol and other molecules comprising a core comprising an inorganic material are reported. Methods for producing nanoscale bound bilayers comprising the depletion of detergents from intermediate mixtures within about one hour of obtaining the mixture are reported in WO 2009/097587. In WO 2006/125304 pharmaceutical compositions for treating or preventing coronary artery disease are reported. Compositions encoding apolipoproteins that are related to lipid metabolism and cardiovascular disease in reported in US 2002/10142953. In WO 2005/084642 an apoprotein-cochelate composition is reported. In WO 2007/137400 a method and compound for the treatment of valvular stenosis is reported. Pharmaceutical formulations, methods and dosing regimens for the treatment and prevention of acute coronary syndromes are reported in WO 2005/041866.

In U.S. Pat. No. 6,287,590 a peptide/lipid complex formation by co-lyophilization is reported. Apolipoprotein A-I agonists and their use to treat dyslipidemic disorders is reported in U.S. Pat. No. 6,037,323.

In WO 2009/097587 nanoscale bound bilayers, methods of use and production are reported. The formulations of hydrophobic proteins in an immunogenic composition having improved tolerability is reported in WO 2005/065708. In WO 2006/069371 a method of plasma lipidation tom prevent, inhibit and/or reverse atherosclerosis is reported. Compositions, uses and methods creating reverse micelles for the clarification of biological fluids to obtain undistorted assay of analytes following clarification is reported in U.S. Pat. No. 4,608,347.

### SUMMARY OF THE INVENTION

Herein is reported a method for producing a lipid particle, wherein the lipid particle is formed in the presence of a synthetic detergent. It has been found that lipid particles can be formed in the presence of a synthetic detergent such as Zwittergent. The use of a synthetic detergent e.g. avoids the use of animal derived components.

Herein is also reported a method for producing a lipid particle comprising a protein. It has been found that lipid particles can be formed starting from a solution comprising the denatured protein by rapid dilution into a solution comprising at least one lipid and a detergent. With this method a preceding naturation step can be omitted and, thus, with the method as reported herein a faster production of lipid particles is possible.

One aspect as reported herein is a method for producing a lipid particle, wherein the lipid particle is formed in the presence of a synthetic detergent.

In one embodiment the synthetic detergent is Zwittergent. In another embodiment the Zwittergent is Zwittergent 3-8 or Zwittergent 3-10.

In one embodiment the method comprises the following:
i) providing a first solution comprising denatured polypeptide,
ii) adding the first solution to a second solution comprising at least one lipid and a synthetic detergent, which does not comprise the polypeptide, i.e. which is free of the polypeptide, and
iii) removing the detergent from the solution obtained in ii) and thereby producing a lipid particle.

In another embodiment the method comprises the following:
i) providing a solution comprising native polypeptide,
ii) adding a lipid and a synthetic detergent to the solution of i), and
iii) removing the detergent from the solution obtained in ii) and thereby producing a lipid particle.

Another aspect reported herein is a method for producing a lipid particle comprising:
i) providing a first solution comprising denatured protein,
ii) adding the first solution to a second solution comprising at least one lipid and a detergent but which does not comprise the protein, and
iii) removing the detergent from the solution obtained in ii) and thereby producing a lipid particle.

In one embodiment the polypeptide is an apolipoprotein. In another embodiment the apolipoprotein is a purified apolipoprotein.

In one embodiment the apolipoprotein has an amino acid sequence selected from the amino acid sequences of SEQ ID NO: 01, 02, 04 to 52, 66 and 67 or comprises at least a contiguous fragment comprising at least 80% of the amino acid sequence of SEQ ID NO: 01, 02, 04 to 52, 66 and 67.

In one embodiment the apolipoprotein has an amino acid sequence or is at least a contiguous fragment of at least 80% of an amino acid sequence selected from SEQ ID NO: 01, 02, 04 to 52, 66 or 67.

In one embodiment the apolipoprotein is an apolipoprotein A-I. In one embodiment the apolipoprotein A-I is human apolipoprotein A-I. In a further embodiment the apolipoprotein is a tetranectin-apolipoprotein A-I that has the amino acid sequence of SEQ ID NO: 01, or SEQ ID NO: 02, or SEQ ID NO: 66, or SEQ ID NO: 67.

In one embodiment the apolipoprotein has the amino acid sequence of SEQ ID NO: 06 with a mutation selected from R151C and R197C.

In one embodiment the at least one lipid is selected from phospholipids, fatty acids and steroid lipids.

In one embodiment the at least one lipid is at least two lipids, optionally selected independently of each other from phospholipids, fatty acids and steroid lipids. In another embodiment the at least one lipid is of from one to four lipids, i.e. it is selected from the group comprising one lipid, two lipids, three lipids, and four lipids.

In one embodiment the second solution comprises a phospholipid, a lipid, and a detergent.

In one embodiment the second solution is consisting of a phospholipid, a lipid, a detergent and a buffer salt.

In one embodiment the lipids are two different phospholipids. In another embodiment the lipids are two different phosphatidylcholines. In another embodiment the first phosphatidylcholine and the second phosphatidylcholine differ in one or two fatty acid residues or fatty acid residue derivatives which are esterified to the glycerol backbone of the phosphatidylcholine. In one embodiment the first phosphatidylcholine is POPC and the second phosphatidylcholine is DPPC.

In one embodiment of the methods as reported herein the first solution is substantially free of lipid particles.

In one embodiment the method comprises after ii) and prior to iii) the following iia) incubating the solution obtained in ii). In one embodiment the incubating and/or removing is at a temperature of from 4° C. to 45° C. In one embodiment the incubating is for about 0.5 hours to about 20 hours, in another embodiment for about 12 hours to about 20 hours. In one embodiment the incubating is for about 16 hours.

In one embodiment the detergent is a detergent with a high CMC. In another embodiment the detergent is a detergent with a CMC of at least 5 mM. In another embodiment the detergent is a detergent with a CMC of at least 10 mM.

In one embodiment the concentration of the detergent is at least 0.5×CMC in the second solution.

In one embodiment the removing is by diafiltration or dialysis or adsorption.

In one embodiment the adsorption is selected from affinity or hydrophobic chromatography.

In one embodiment the first solution has a first volume, the second solution has a second volume, the apolipoprotein in the first solution is present at a defined concentration, and the lipids and the detergent in the second solution are each present at a defined concentration wherein in ii) the concentration of the apolipoprotein, of the lipids, and of the detergent is changed/reduced allowing the formation of a lipid particle.

In one embodiment the first solution has a first volume, the second solution has a second volume, the protein in the first solution has a defined concentration, and the lipids and the detergent in the second solution each have a defined concentration, and in ii) the concentration of the apolipoprotein, of the lipids and of the detergent is changed/reduced allowing the formation of a lipid particle.

In one embodiment the method comprises the following:
iv) purifying the lipid particle and thereby producing a lipid particle.

In one embodiment the second method comprises the following ii):
ii) adding the at least one lipid and the synthetic detergent to the solution of i) and adjusting the concentrations and concentration ratios of the lipid, the detergent and the apolipoprotein.

One aspect as reported herein is a lipid particle obtained by a method as reported herein.

One aspect as reported herein is a pharmaceutical composition comprising a lipid particle comprising apolipoprotein obtained with a method as reported herein as well as the use of a lipid particle as reported herein for the manufacture of a medicament for the treatment of arteriosclerosis.

### DESCRIPTION OF THE SEQUENCE LISTING

### SEQ ID NO: 01 Tetranectin-apolipoprotein A-I (1).

### SEQ ID NO: 02 Tetranectin-apolipoprotein A-I (2).

### SEQ ID NO: 03 Peptide.

SEQ ID NO: 04 Apolipoprotein A-I mimetic (1).
SEQ ID NO: 05 Apolipoprotein A-I mimetic (2).
SEQ ID NO: 06 Human apolipoprotein A-I.
SEQ ID NO: 07 Human apolipoprotein A-II.
SEQ ID NO: 08 Human apolipoprotein A-IV.
SEQ ID NO: 09 Human apolipoprotein A-V.
SEQ ID NO: 10 Human apolipoprotein C-I.
SEQ ID NO: 11 Human apolipoprotein C-II.
SEQ ID NO: 12 Human apolipoprotein C-III.
SEQ ID NO: 13 Human apolipoprotein C-IV.
SEQ ID NO: 14 Human apolipoprotein D.
SEQ ID NO: 15 Human apolipoprotein E.
SEQ ID NO: 16 Human apolipoprotein F.
SEQ ID NO: 17 Human apolipoprotein H.
SEQ ID NO: 18 Human apolipoprotein L-I.
SEQ ID NO: 19 Human apolipoprotein L-II.
SEQ ID NO: 20 Human apolipoprotein L-III.
SEQ ID NO: 21 Human apolipoprotein L-IV.
SEQ ID NO: 22 Human apolipoprotein L-V.
SEQ ID NO: 23 Human apolipoprotein L-VI.
SEQ ID NO: 24 Human apolipoprotein M.
SEQ ID NO: 25 Human apolipoprotein O.
SEQ ID NO: 26 Human apolipoprotein OL.
SEQ ID NO: 27 Human apolipoprotein clus.

### SEQ ID NO: 28 Apolipoprotein.

### SEQ ID NO: 29 Apolipoprotein.

### SEQ ID NO: 30 Apolipoprotein.

### SEQ ID NO: 31 Apolipoprotein.

### SEQ ID NO: 32 Apolipoprotein.

### SEQ ID NO: 33 Apolipoprotein.

### SEQ ID NO: 34 Apolipoprotein.

### SEQ ID NO: 35 Apolipoprotein.

### SEQ ID NO: 36 Apolipoprotein.

### SEQ ID NO: 37 Apolipoprotein.

### SEQ ID NO: 38 Apolipoprotein.

### SEQ ID NO: 39 Apolipoprotein.

### SEQ ID NO: 40 Apolipoprotein.

### SEQ ID NO: 41 Apolipoprotein.

### SEQ ID NO: 42 Apolipoprotein.

### SEQ ID NO: 43 Apolipoprotein.

### SEQ ID NO: 44 Apolipoprotein.

### SEQ ID NO: 45 Apolipoprotein.

### SEQ ID NO: 46 Apolipoprotein.

### SEQ ID NO: 47 Apolipoprotein.

### SEQ ID NO: 48 Apolipoprotein.

### SEQ ID NO: 49 Apolipoprotein.

### SEQ ID NO: 50 Apolipoprotein.

### SEQ ID NO: 51 Apolipoprotein.

### SEQ ID NO: 52 Apolipoprotein.

SEQ ID NO: 53 Human tetranectin trimerization domain.
SEQ ID NO: 54 Shortened human tetranectin trimerization domain.
SEQ ID NO: 55 Human interferon fragment.

### SEQ ID NO: 56 Hexahistidine tag.

SEQ ID NO: 57 Fusion protein.

### SEQ ID NO: 58 Primer N1.

### SEQ ID NO: 59 Primer N2.

SEQ ID NO: 60 IgA protease cleavage site.
SEQ ID NO: 61 IgA protease cleavage site.
SEQ ID NO: 62 IgA protease cleavage site.
SEQ ID NO: 63 IgA protease cleavage site.
SEQ ID NO: 64 IgA protease cleavage site.
SEQ ID NO: 65 IgA protease cleavage site.

### SEQ ID NO: 66 Tetranectin-apolipoprotein A-I.

SEQ ID NO: 67 Tetranectin-apolipoprotein A-I with his-tag.

### SEQ ID NO: 68 to 105 Linker.

### DESCRIPTION OF THE FIGURES

FIG. 1 Results of in vivo rabbit studies conducted with five lipid particles differing in their lipid composition. Top: cholesterol mobilization and, thus, efficacy could be shown for all prepared batches. Bottom: Increase of liver enzyme was noticed for lipid particles generated by the use of DPPC as single phospholipid.

FIG. 2 SEC-MALLS analysis of lipid particles of POPC and apolipoprotein according to the current invention; molar ratios 1:20 to 1:160.

FIG. 3 Impact of DPPC and POPC on LCAT activity.

FIG. 4 Initial velocity of cholesterol esterification in lipid particles containing POPC and/or DPPC.

FIG. 5 Cholesterol efflux to THP-1 derived foam cells in cells not primed with a RXR-LXR agonist.

FIG. 6 Cholesterol efflux to THP-1 derived foam cells after ABCA-I pathway activation using an RXR-LXR agonist.

FIG. 7 Time dependent plasma concentration of different apolipoprotein compositions.

FIG. 8 Time and concentration course of cholesterol mobilization and esterification in plasma.

FIG. 9 Comparison of liver enzyme release by different compositions comprising apolipoprotein according to the invention in mice after a single i.v. injection of 100 mg/kg.

FIG. 10 In vivo rabbit study—spontaneous hemolysis in plasma.

FIG. 11 Analytical SEC of lipid particles using 250 mM Tris-HCl, 140 mM NaCl, pH 7.5.

FIG. 12 Analytical SEC of lipid particles using 50 mM K₂HPO₄, 250 mM arginine hydrochloride, 7.5% trehalose at pH 7.5.

FIG. 13 Native PAGE of lipid particles of POPC and tetranectin-apolipoprotein A-I in molar ratios of from 1:20 to 1:320 (lane 1: native Marker; lane 2: molar ratio 1:320; lane 3: molar ratio 1:160; lane 4: molar ratio 1:80; lane 5: molar ratio 1:80 (f/t); lane 6: molar ratio 1:40; lane 7: molar ratio 1:20; lane 8: apolipoprotein (forming hexamers)).

FIG. 14 SEC-MALLS analysis of lipid particles of POPC and tetranectin-apolipoprotein A-I in molar ratios of from 1:20 to 1:160.

FIG. 15 Superposition of SEC chromatograms (UV280 signal) of lipid particle of POPC and tetranectin-apolipoprotein A-I.

FIG. 16 SEC-MALLS analysis of a lipid particle of POPC and tetranectin-apolipoprotein A-I obtained at a molar ratio of 1:40.

FIG. 17 Native PAGE of lipid particles of DPPC and tetranectin-apolipoprotein A-I obtained with molar ratios of from 1:20 to 1:100 (1: molecular weight marker; 2: tetranectin-apolipoprotein A-I without lipid; 3: 1:20; 4: 1:40; 5: 1:60; 6: 1:80; 7: 1:100).

FIG. 18 SEC-MALLS analysis (UV280 signal) of a lipid particle of a mixture of POPC:DPPC=3:1 and tetranectin-apolipoprotein A-I obtained at molar ratios of from 1:60 (uppermost curve) to 1:100 (lowest curve).

FIG. 19 Native PAGE SDS of a lipid particle of tetranectin-apolipoprotein A-I using cholate, Zwittergent 3-8, 3-10 and 3-12. Lane 1 on each gel: pure apolipoprotein; lane 2 on each gel: 0.1×CMC cholate lipidated sample as references.

FIG. 20 SEC-MALLS protein conjugate analysis of lipid particle of tetranectin-apolipoprotein A-I using 3×CMC Zwittergent 3-8 and POPC (molar ratio apolipoprotein:phospholipid=1:60).

FIG. 21 SEC-MALLS protein conjugate analysis of lipid particle of tetranectin-apolipoprotein A-I using 2×CMC Zwittergent 3-10 and POPC (molar ratio apolipoprotein:phospholipid=1:60).

FIG. 22 SEC-MALLS protein conjugate analysis of lipid particle of tetranectin-apolipoprotein A-I using POPC. Upper: lipid particle formed from native tetranectin-apolipoprotein A-I; lower: lipid particle formed from denatured tetranectin-apolipoprotein A-I.

FIG. 23 Results of in vivo rabbit studies performed with tetranectin-apolipoprotein A-I lipidated with DMPC (1:100) (di myristoyl phosphatidylcholine) (a) and not lipidated in PBS (b).

FIG. 24 SE-HPLC chromatogram of lipid particles containing wild-type apolipoprotein A-I (A) and tetranectin-apolipoprotein A-I as reported herein (B) stored at 5° C. and 40° C.

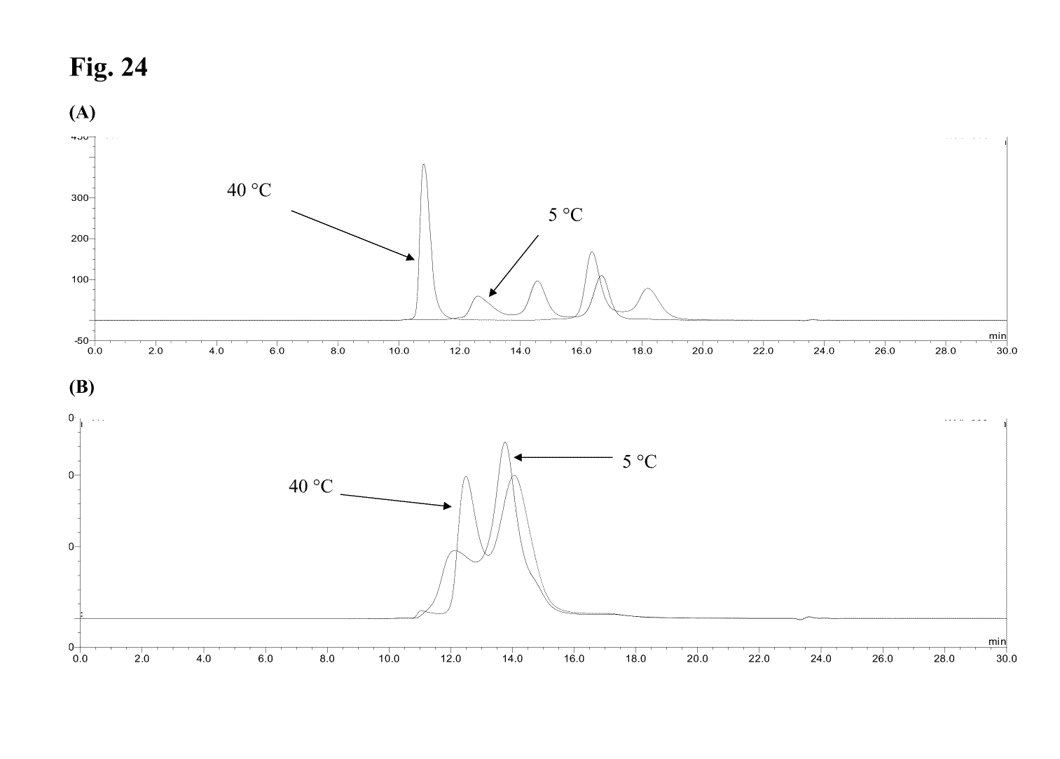

### DETAILED DESCRIPTION OF THE INVENTION

### Definitions

The term “apolipoprotein” denotes a protein that is comprised in a lipid or lipoprotein particle, respectively.

The term “apolipoprotein A-I” denotes an amphiphilic, helical polypeptide with protein-lipid and protein-protein interaction properties. Apolipoprotein A-I is synthesized by the liver and small intestine as prepro-apolipoprotein of 267 amino acid residues which is secreted as a pro-apolipoprotein that is cleaved to the mature polypeptide having 243 amino acid residues. Apolipoprotein A-I is consisting of 6 to 8 different amino acid repeats consisting each of 22 amino acid residues separated by a linker moiety which is often proline, and in some cases consists of a stretch made up of several residues. An exemplary human apolipoprotein A-I amino acid sequence is reported in GenPept database entry NM-000039 or database entry X00566; GenBank NP-000030.1 (gi 4557321). Of human apolipoprotein A-I (SEQ ID NO: 06) naturally occurring variants exist, such as P27H, P27R, P28R, R34L, G50R, L84R, D113E, A-A119D, D127N, deletion of K131, K131M, W132R, E133K, R151C (amino acid residue 151 is changed from Arg to Cys, apolipoprotein A-I-Paris), E160K, E163G, P167R, L168R, E171V, P189R, R197C (amino acid residue 173 is change from Arg to Cys, apolipoprotein A-I-Milano) and E222K. Also included are variants that have conservative amino acid modifications.

In one embodiment the tetranectin-apolipoprotein A-I comprises a fragment of the cleavage site of Immunoglobulin A protease (IgA protease). The recognition sites known from IgA proteases comprise the following sequences with “↓” denoting the position of the cleaved bond:
Pro-Ala-Pro↓Ser-Pro (SEQ ID NO: 61)
Pro-Pro↓Ser-Pro (SEQ ID NO: 62)
Pro-Pro↓Ala-Pro (SEQ ID NO: 63)
Pro-Pro↓Thr-Pro (SEQ ID NO: 64)
Pro-Pro↓Gly-Pro (SEQ ID NO: 65),
   wherein the first three are more frequently chosen and cleaved.

The term “apolipoprotein mimic” denotes a synthetic polypeptide that mimics the function of the respective apolipoprotein. For example an “apolipoprotein A-I mimic” is a synthetic polypeptide that shows comparable biological function with respect to removal of cholesterol, i.e. reverse cholesterol efflux, as the natural apolipoprotein A-I. In one embodiment the apolipoprotein A-I mimic comprises at least one amphiphilic alpha-helix with positively charged amino acid residues clustered at a hydrophobic-hydrophilic interface and negatively-charged amino acid residues clustered at a center of a hydrophilic face. In order to mimic the function of apolipoprotein A-I the apolipoprotein mimic comprise a repeat polypeptide of from 15 to 29 amino acid residues, in one embodiment of 22 amino acid residues (PVLDEFREKLNEELEALKQKLK (SEQ ID NO: 04); PVLDLFRELLNELLEALKQKLK (SEQ ID NO: 05)).

The term “at least one” denotes one, two, three, four, five, six, seven, eight, nine, ten or more. The term “at least two” denotes two, three, four, five, six, seven, eight, nine, ten or more.

The term “cardiovascular disease” in general denotes a disease or condition with respect to heart or blood vessels, such as arteriosclerosis, coronary heart disease, cerebrovascular disease, aortoiliac disease, ischemic heart disease or peripheral vascular disease. Such a disease may not be discovered prior to an adverse event as a result of the disease, such as myocardial infarct, stroke, angina pectoris, transient ischemic attacks, congestive heart failure, aortic aneurysm, mostly resulting in death of the subject.

The term “cholate” denotes 3α,7α,12α-trihydroxy-5β-cholan-24-oic acid or a salt thereof, especially the sodium salt. The formation of lipid particles may be performed by incubating the apolipoprotein with detergent solubilized lipids at their respective transition temperature.

The term “critical micelle concentration” and its abbreviation “CMC”, which can be used interchangeably, denotes the concentration of surfactants or detergents above which the addition of further surfactant or detergent does not further reduce the surface tension of the solution.

The term “conservative amino acid modification” denotes modifications of the amino acid sequence which do not affect or alter the characteristics of the lipid particle or the apolipoprotein according to the invention. Modifications can be introduced by standard techniques known in the art, such as site-directed mutagenesis and PCR-mediated mutagenesis. Conservative amino acid modifications include ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), non-polar side chains (e.g. alanine, valine, leucine, isoleucine, proline, phenylalanine, methionine), beta-branched side chains (e.g. threonine, valine, isoleucine), and aromatic side chains (e.g. tyrosine, phenylalanine, tryptophan, histidine). A “variant” protein, refers therefore herein to a molecule which differs in amino acid sequence from a “parent” protein's amino acid sequence by up to ten, in one embodiment from about two to about five, additions, deletions, and/or substitutions Amino acid sequence modifications can be performed by mutagenesis based on molecular modeling as described by Riechmann, L., et al., Nature 332 (1988) 323-327, and Queen, C., et al., Proc. Natl. Acad. Sci. USA 86 (1989) 10029-10033.

The term “detergent” denotes a surface active chemical substance. In one embodiment the detergent is selected from sugar-based detergents, polyoxyalkylene-based detergents, bile-salt based detergents, synthetic detergents or a combination thereof. The term “sugar-based detergent” denotes a detergent selected from n-octyl-beta-D-glucopyranoside, n-nonyl-beta-D-glucopyranoside, n-dodecyl-beta-D-maltopyranoside, or 5-cyclohexylpentyl-beta-D-maltopyranoside, and derivatives thereof. The term “bile-salt based detergent” denotes a detergent selected from sodium cholate, potassium cholate, lithium cholate, 3-[(3-chloramidopropyl)dimethylammonio]-yl-propane sulfonate (CHAPS), 3-[(3-chloramidopropyl)dimethylammonio]-2-hydroxyl propane sulfonate (CHAPSO), and derivatives thereof. The term “polyoxyalkylene-based detergent” denotes a detergent selected from Tween 20, Triton X-100, Pluronic F68, and a derivatives thereof. The term “synthetic detergents” denotes a detergent selected from Zwittergent 3-6, Zwittergent 3-8, Zwittergent 3-10, Zwittergent 3-12, and derivatives thereof.

The term “high density lipoprotein particle” or its abbreviation “HDL particle”, which can be used interchangeably, denotes a lipid-protein-complex comprising as main proteinaceous compound apolipoprotein A-I.

The term “immunoassay” denotes standard solid-phase immunoassays with monoclonal antibodies involving the formation of a complex between an antibody adsorbed/immobilized on a solid phase (capture antibody), the antigen, and an antibody to another epitope of the antigen conjugated with an enzyme (tracer antibody). Thus, a sandwich is formed: solid phase-capture antibody-antigen-tracer antibody. In the reaction catalyzed by the sandwich, the activity of the antibody-conjugated enzyme is proportional to the antigen concentration in the incubation medium. The standard sandwich method is also called double antigen bridging immunoassay because capture and tracer antibodies bind to different epitopes of the antigen. Other types of assays are radioimmunoassay, fluorescence immunoassays and enzyme-linked immunoassays. Methods for carrying out such assays as well as practical applications and procedures are known to a person of skill in the art. The immunoassays can be performed as homogeneous or heterogeneous immunoassay.

The term “increase lipid efflux” and grammatical equivalents thereof denotes an increased level and/or rate of lipid efflux, promoting lipid efflux, enhancing lipid efflux, facilitating lipid efflux, upregulating lipid efflux, improving lipid efflux, and/or augmenting lipid efflux from cells or plaques. In one embodiment, the lipid efflux comprises efflux of phospholipid, triglyceride, cholesterol, and/or cholesterol ester.

The term “DMPC” denotes the phospholipid dimyristoyl phosphatidylcholine.

The term “DPPC” denotes the phospholipid 1,2-di-palmitoyl-sn-glycero-3-phosphatidylcholine also referred to as 1,2-dipalmitoyl-phosphatidylcholine.

The term “multimer” denotes a complex consisting of two or more monomers. A multimer is formed by non-covalent interactions between the monomers. Each monomer comprises a multimerization domain. In one embodiment the multimer comprises 2 or 3 monomers. In another embodiment the multimerization domains interact via non-covalent interactions between the individual multimerization domains comprised in each monomer. The term “multimerization domain” denotes amino acid sequences capable of covalently or non-covalently associating two or more monomeric molecules. A multimerization domain is capable of interacting with multimerization domains of different, similar, or identical amino acid sequence. In one embodiment the multimerization domain is the tetranectin trimerising structural element or a derivative thereof that has an amino acid sequence that is at least 68% identical with the consensus amino acid sequence of SEQ ID NO: 53. In one embodiment the cysteine residue at position 50 of SEQ ID NO: 53 is substituted by a different amino acid residue, in another embodiment by a serine residue, or a threonine residue, or a methionine residue. Polypeptides comprising a multimerization domain can associate with one or more other polypeptides also comprising a multimerization domain. The multimer formation can be initiated simply by mixing the polypeptides under suitable conditions. In another embodiment the multimerization domain has the amino acid sequence of SEQ ID NO: 53 wherein of from 1 to 10 residues have been deleted from or added to the N- or C-terminus of the amino acid sequence. In a further embodiment the multimerization domain has an amino acid sequence of SEQ ID NO: 53 wherein six or nine amino acid residues have been deleted from the N-terminus of the amino acid sequence. In still another embodiment the multimerization domain has an amino acid sequence of SEQ ID NO: 53 wherein the N-terminal amino acid residue L or the N-terminal amino acid residues C and L have been deleted. In one embodiment the multimerization domain is the tetranectin trimerising structural element and has the amino acid sequence of SEQ ID NO: 54. The multimer is in one embodiment a homomer.

The multimers may be homomers or heteromers, since different apolipoproteins comprising a multimerization domain can be combined to be incorporated into the multimer. In one embodiment the multimer is a trimeric homomer.

According to one embodiment the multimerization domain is obtained from tetranectin. In one embodiment the multimerization domain comprises the tetranectin trimerising structural element that has an amino acid sequence of SEQ ID NO: 54. The trimerising effect of the tetranectin trimerising structural element is caused by a coiled coil structure which interacts with the coiled coil structure of two other tetranectin trimerising structural elements to form a trimer. The tetranectin trimerising structural element may be obtained from human tetranectin, from rabbit tetranectin, from murine tetranectin, or from C-type lectin of shark cartilage. In one embodiment the tetranectin trimerising structural element comprises a sequence having at least 68%, or at least 75%, or at least 81%, or at least 87%, or at least 92% identity with the consensus sequence of SEQ ID NO 53.

The term “non-covalent interactions” denotes non-covalent binding forces such as ionic interaction forces (e.g. salt bridges), non-ionic interaction forces (e.g. hydrogen-bonds), or hydrophobic interaction forces (e.g. van-der-Waals forces or π-stacking interactions).

The term “phase transition temperature” denotes the temperature required to induce a change in the lipid physical state from the ordered gel phase, where the hydrocarbon chains are fully extended and closely packed, to the disordered liquid crystalline phase, where the hydrocarbon chains are randomly oriented and fluid. The formation of the lipid particles may be carried out at or above the phase transition temperature of the phospholipids/phospholipid mixtures used. The phase transition temperature of some phosphatidylcholines and mixtures thereof are listed in the following Table 1.

TABLE 1
Transition temperatures of pure phosphatidylcholines
and phosphatidylcholine mixtures.

| phospholipid molar ratio | phase transition temperature |
|---|---|
| POPC | 4° C. (−3° C.) |
| DPPC | 41° C. |
| DPPC:POPC 3:1 | 34° C. |
| DPPC:POPC 1:1 | 27° C. |
| DPPC:POPC 1:3 | 18° C. |

The term “phosphatidylcholine” denotes a molecule consisting of one glycerol moiety, two carboxylic acid moieties and one phosphocholine moiety, wherein the glycerol moiety is covalently bound to the other moieties each by a ester bond, i.e. two carboxylic ester bonds and one phosphoric ester bond, whereby the phosphoric ester bond is either to the 1-hydroxyl group or the 3-hydroxyl group of the glycerol moiety. The term “carboxylic acid moiety” denotes an organic moiety comprising at least one acyl group (R—C(O)O). The phosphatidylcholine may be of any kind or source. In one embodiment the phosphatidylcholine is selected from egg phosphatidylcholine, soybean phosphatidylcholine, dipalmitoyl phosphatidylcholine, dimyristoyl phosphatidylcholine, distearoyl phosphatidylcholine, dilauryl phosphatidylcholine, dipalmitoyl phosphatidylcholine, 1-myristoyl-2-palmitoyl phosphatidylcholine, 1-palmitoyl-2-myristoyl phosphatidylcholine, 1-palmitoyl-2-stearoyl phosphatidylcholine, 1-stearoyl-2-palmitoyl phosphatidylcholine, dioleoyl phosphatidylcholine, 1-palmitoyl-2-oleoyl phosphatidylcholine, 1-oleoyl-2-palmitoyl phosphatidylcholine, and an analogues and derivatives thereof.

All phospholipids as used herein may be derived from any source, i.e. (where appropriate) from soybean, milk, egg or even inner organs of animals excluding humans, they may be derived from natural origin, or semi-synthetic or even fully synthetic.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The term “POPC” denotes the phospholipid 1-palmitoyl-2-oleoyl-sn-glycero-3-phosphatidylcholine also referred to as 1-palmitoyl-2-oleoyl-phosphatidylcholine.

The term “rapid” denotes a process that is completed within at most 10 hours. A rapid dilution is a process in which a first solution is added to a second solution in at most 10 hours. In one embodiment the process is completed in at most 5 hours, in a further embodiment in at most 2 hours.

The term “substantially free” denotes that a solution comprising a protein an one or more lipids contains less than 5% (w/w) lipid particles, less than 2.5% lipid particles, less than 1% lipid particles, or less than 0.5% lipid particles.

The term “variant” includes also variants of an apolipoprotein or an apolipoprotein mimic as reported herein wherein in the variants the amino acid sequence of the respective apolipoprotein or apolipoprotein mimic comprises one or more amino acid substitution, addition or deletion. The modification may increase or decrease the affinity of the apolipoprotein for an apolipoprotein receptor or an apolipoprotein converting enzyme, or may increase the stability of the apolipoprotein variant compared to the respective apolipoprotein, or may increase the solubility of the apolipoprotein variant compared to the respective apolipoprotein in aqueous solutions, or may increase the recombinant production of the apolipoprotein variant compared to the respective apolipoprotein in/by host cells.

### Reported Herein

It has been found that lipid particles and be formed by using as sole detergent a synthetic detergent. It is advantageous to use a synthetic detergent with a CMC of at least 10 mM. Already at a concentration of 0.5×CMC, i.e. at halve the concentration that is required for micelle formation, the formation of lipid particles can be detected. Thus, only a small amount of detergent is necessary for the formation of lipid particles. This has advantages such as a reduced risk of adverse effects by in vivo application of this lipid particle as a smaller concentration of detergent is required for the formation of the lipid particle. In addition, in combination with an improved method for producing lipid particles directly from a solution, which is containing a denatured protein but no detergent and no lipid, by rapid dilution into a solution containing a detergent and at least one lipid the use of a synthetic detergent is even more advantageous.

It has also been found that lipid particles and be formed directly from a solution containing a denatured protein but no detergent and no lipid by rapid dilution into a solution containing a detergent and at least one lipid but no protein. The generally required naturation step can be omitted, thus, providing for more simple and robust method for the production of lipid particles. Additionally a more homogeneous lipid particle is formed.

### Method for the Production of Lipid Particles

Herein is reported a method for producing a lipid particle, wherein the lipid particle is formed in the presence of a synthetic detergent. It has been found that lipid particles can be formed in the presence of solely a synthetic detergent wherein the synthetic detergent has a CMC of at least 5 mM. The use of a synthetic detergent e.g. avoids the use of animal derived components and allows the formation of lipid particles at low concentrations of detergent.

One aspect as reported herein is a method for producing a lipid particle which comprises a polypeptide and a lipid, wherein the lipid particle is formed in the presence of a synthetic detergent.

In one embodiment the synthetic detergent has a CMC of at least 10 mM. In another embodiment the synthetic detergent has a CMC of at least 35 mM.

In one embodiment the synthetic detergent allows for the formation of a lipid particle at a concentration of 0.5×CMC of the synthetic detergent.

In one embodiment the synthetic detergent is Zwittergent. In another embodiment the Zwittergent is Zwittergent 3-8 or Zwittergent 3-10.

In one embodiment the method comprises the following:
i) providing a first solution comprising the denatured polypeptide,
ii) adding the first solution to a second solution, which comprises at least one lipid and a synthetic detergent but which is free of the polypeptide, and
iii) removing the detergent from the solution obtained in ii) and thereby producing a lipid particle.

In one embodiment the method comprises the following:
i) providing a first solution comprising denatured apolipoprotein,
ii) adding the first solution to a second solution comprising at least one lipid and a synthetic detergent but no apolipoprotein, and
iii) removing the detergent from the solution obtained in ii) and thereby producing a lipid particle.

In one embodiment the method comprises the following:
i) providing a solution comprising the native polypeptide,
ii) adding at least one lipid and a synthetic detergent to the solution of i), and
iii) removing the detergent from the solution obtained in ii) and thereby producing a lipid particle.

In another embodiment the method comprises the following:

i) providing a solution comprising native apolipoprotein,
ii) adding at least one lipid and a synthetic detergent to the solution of i), and
iii) removing the detergent from the solution obtained in ii) and thereby producing a lipid particle.

In another embodiment there is provided a method for producing a lipid particle, which comprises a protein, with the method comprising the following:
i) providing a first solution comprising denatured protein,
ii) adding the first solution to a second solution, which comprises a lipid and a detergent but no protein, i.e. which is free of the protein, and
iii) removing the detergent from the solution obtained in ii) and thereby producing a lipid particle.

In another embodiment the method for producing a lipid particle, which comprises an apolipoprotein, wherein the method comprises:
i) providing a first solution comprising denatured apolipoprotein,
ii) adding the first solution to a second solution, which comprises a lipid and a detergent but no apolipoprotein, and
iii) removing the detergent from the solution obtained in ii) and thereby producing a lipid particle.

In one embodiment the second solution comprises at least two different lipids independently of each other selected from phospholipids, fatty acids and steroid lipids. In another embodiment the at least two different lipids are two different phosphatidylcholines. In another embodiment the first phosphatidylcholine is POPC and the second phosphatidylcholine is DPPC.

In one embodiment the detergent is selected from cholic acid, Zwittergent or a salt thereof.

A number of different methods for the production of lipid particles from naturally occurring or recombinantly produced polypeptides, such as e.g. apolipoprotein A-I or delipidated apolipoprotein A-I derived from human HDL particles, have been reported. Therein, for example, an aqueous mixture of phospholipids such as palmitoyl-2-oleoyl-sn-glycero-3-phosphocholine with detergents such as sodium cholate are incubated with purified apolipoprotein A-I, wherein the apolipoprotein A-I is employed in a non-denatured form. The detergent is removed after the formation of the lipid particle by dialysis or diafiltration.

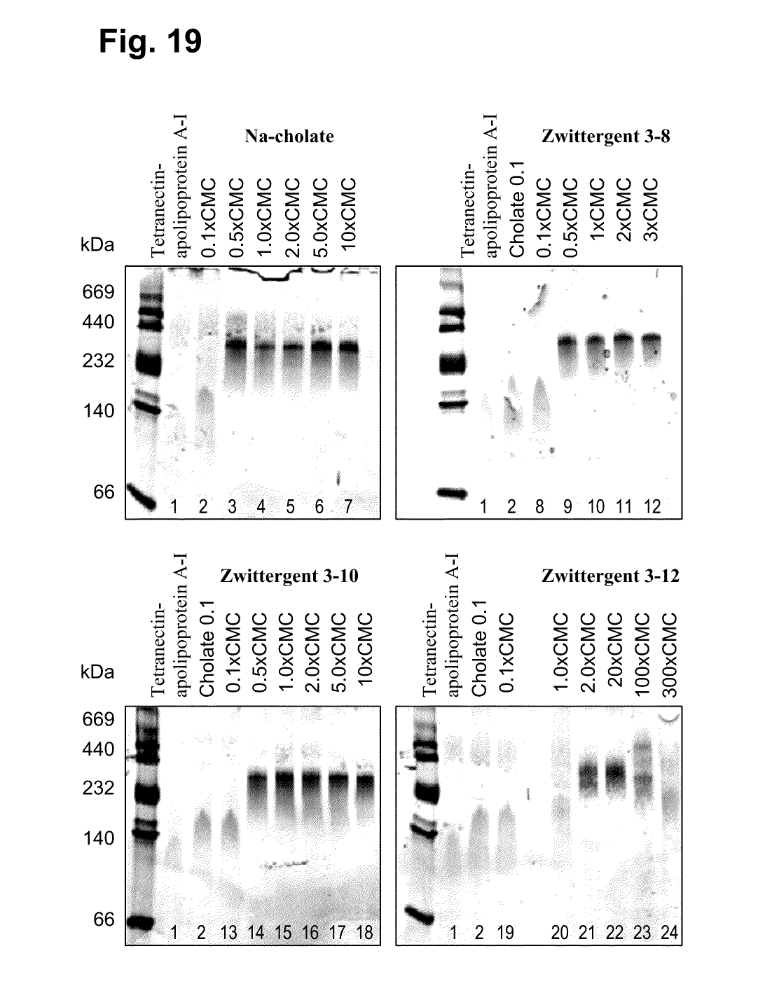

It has now been found that for the formation of a lipid particle a synthetic detergent with a CMC of at least 5 mM can be used. With such a synthetic detergent on the one hand a low detergent concentration is required for the formation of a lipid particle and on the other hand a more homogeneous product is obtained, i.e. a product with less side products. With a synthetic detergent with a lower CMC, such as e.g. Zwittergent 3-12 with a CMC of 2.8 mM, a higher concentration thereof is required for the formation of a lipid particle at all let alone the more heterogeneous product that is formed (see FIG. 19).

A synthetic detergent is neither a detergent that is neither occurring in nature nor isolated from a natural source nor a synthetically produced detergent occurring in nature. Thus, a synthetic detergent is completely designed by man. Examples of synthetic detergent with a CMC of 5 mM or more are Zwittergent 3-8 (n-octyl-N,N-dimethyl-3-ammonio-1-propanesulfonate; CMC=390 mM), Zwittergent 3-10 (n-decyl-N,N-dimethyl-3-ammonio-1-propanesulfonate; CMC=39 mM), Fos-Choline-10 (CMC=11 mM), CHAPS (3-[(3-chloramidopropyl)dimethylammonio]-1-propane sulfonate; CMC=8 mM), CHAPSO (3-[(3-chloramidopropyl)dimethylammonio]-2-hydroxy-1-propane sulfonate; CMC=8 mM), n-Octyl-β-D-Maltopyranoside (CMC=19 mM).

The method as reported herein allows to refold and lipidate completely denatured apolipoprotein A-I in a single step. By using a method as reported herein (i) a lipid particle with improved product quality can be obtained, (ii) the time consuming preconditioning of apolipoprotein A-I can be omitted, and (iii) a large scale processing for biopharmaceutical production is for the first time possible.

The method as reported herein allows to refold and to lipidate completely denatured protein in a single step. By using a method as reported herein a lipid particle with improved product quality can be obtained, the time consuming preconditioning of the protein can be omitted and a large scale processing for biopharmaceutical production is possible for the first time.

The main points which have to be considered for the lipid particle formation process development are i) the requirements for biological activity, and ii) technical requirements directed to the manufacturability of the lipid particle. For example, for the formation of lipid particles comprising an apolipoprotein these requirements point in opposite directions.

From a technical point of view saturated phospholipids containing carboxylic acid moieties with a chain of 16 carbon atoms and shorter would be chosen (e.g. dipalmitoyl-sn-glycero-3-phosphocholine, DPPC; dimyristoyl-sn-glycero-3-phosphocholine, DMPC etc.). In contrast thereto from biological data it can be assumed that non-saturated phospholipids containing carboxylic acid moieties with a chain of at least 16 carbon-atoms (e.g. palmitoyl-2-oleoyl-sn-glycero-3-phosphocholine, POPC; stearoyl-2-oleoyl-sn-glycero-3-phosphocholine, SOPC) are more effective and non-liver toxic.

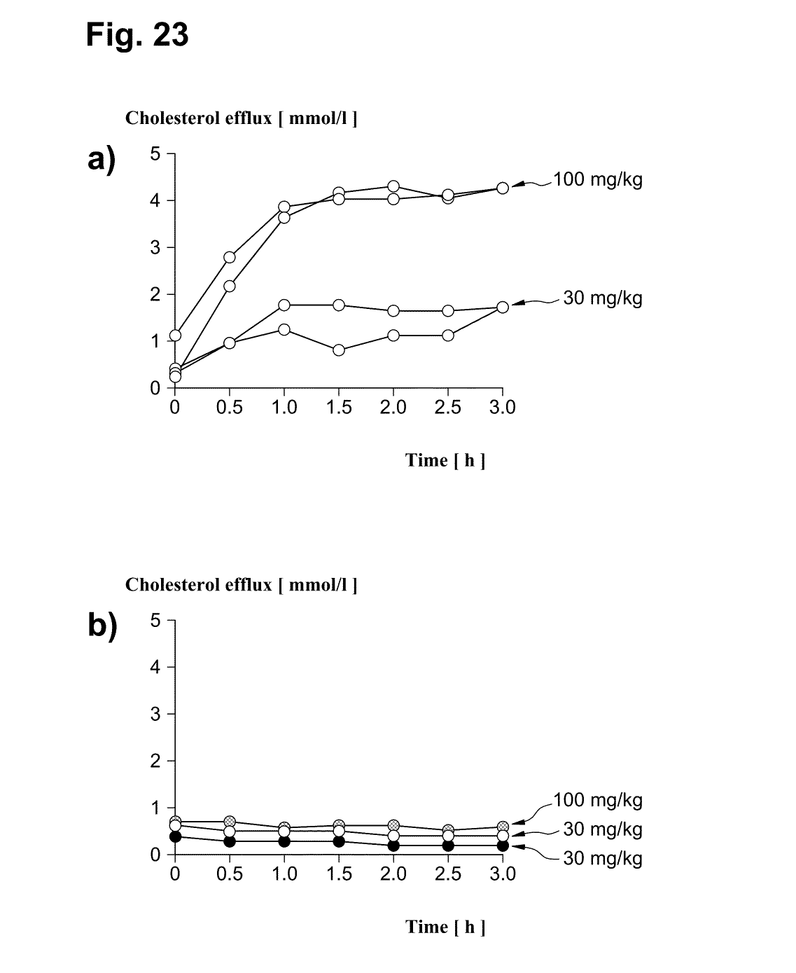

The choice of the combination of lipids determines the efficacy and liver safety of lipid particles comprising apolipoprotein. In in vivo studies of DMPC containing lipid particles using rabbits it has been found that rabbits treated with 30 mg/kg showed severe side effects but survived whereas rabbits treated with 100 mg/kg died. Results clearly indicated that lipidation is needed for cholesterol mobilization and consequently for the efficacy of the molecule (FIG. 23).

In vitro functional tests confirmed that a lipid particle containing a single phosphatidylcholine such as DPPC or POPC activate LCAT.

It was also shown that cholesterol efflux was higher when the lipid particle comprised a combination of different phospholipids.

TABLE 2
Phospholipid combinations differing in their lipid
composition prepared for in vivo rabbit studies.

| phospholipid | | |
|---|---|---|
| molar ratio used | | |
| for producing the | LCAT | cholesterol |
| lipid particle | substrate | efflux |
| POPC | yes | yes |
| POPC:DPPC 3:1 | yes | yes |
| POPC:DPPC 1:1 | yes | yes |
| POPC:DPPC 1:3 | no | yes |
| DPPC | no | yes |

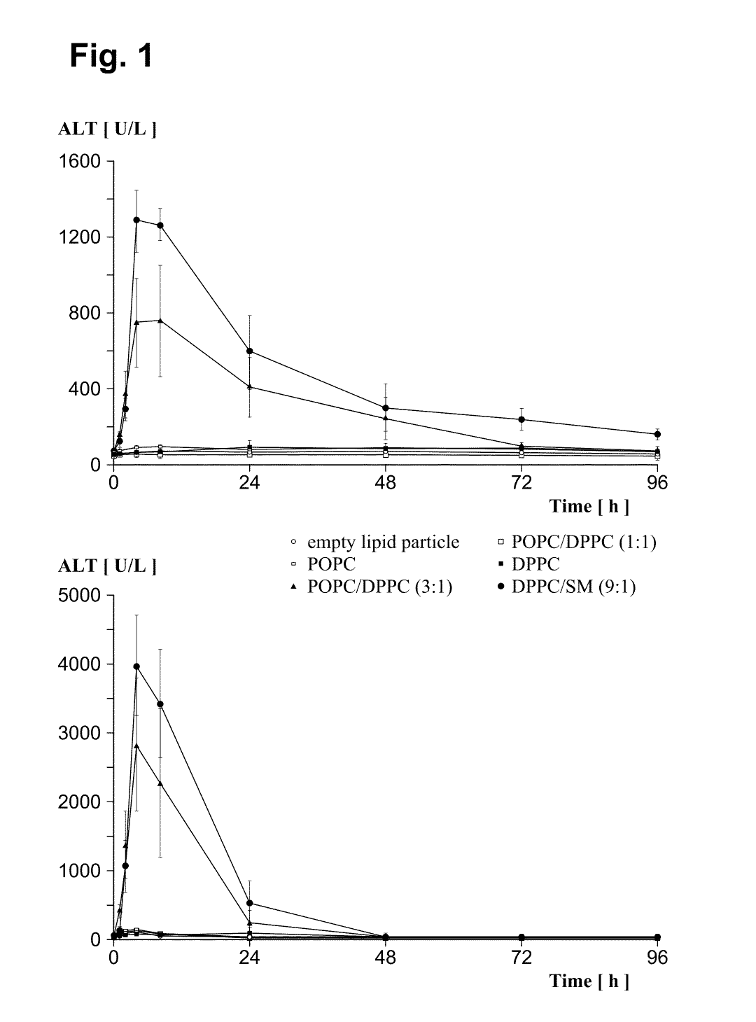

These results were also confirmed by in vivo data demonstrating cholesterol mobilization for all combinations. However, for lipid particles containing only the single phosphatidylcholine DPPC, or the combination of DPPC and sphingomyelin (SM) an increase in liver enzymes can be determined (FIG. 1).

Thus, also an aspect is a lipid particle obtained by a method as reported herein.

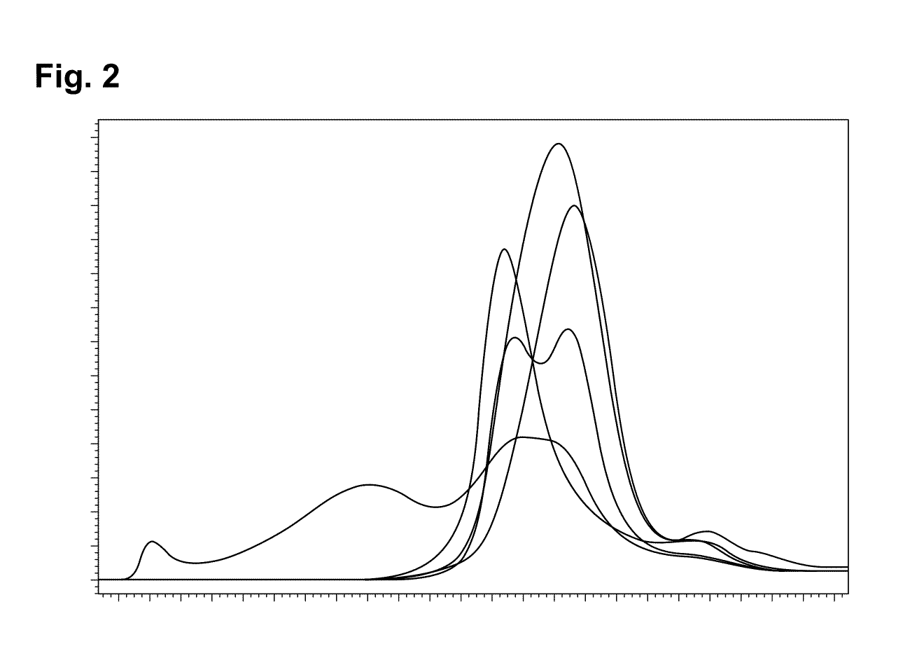

From the technical point of view the formation of lipid particles with pure DPPC is more convenient compared to the formation with pure POPC. The risk of precipitate formation is reduced by using a combination of different phospholipids. Also the phase transition temperature of 41° C. for pure DPPC makes it easier to prepare the lipid particle compared to pure POPC that has a phase transition temperature of 4° C. Also the obtained product is more homogeneous. This can be confirmed by lipid particle analysis via SEC-MALLS, an analytical tool which also allows the determination of the protein-lipid composition (protein-conjugate analysis). In FIG. 2 a chromatogram of samples resolved in a size-exclusion chromatography (UV280 detection) is shown. An inhomogeniety of a sample can be seen by the occurrence of multiple separated or semi-detached peaks.

The number of POPC molecules per apolipoprotein monomer in the lipid particle when pure POPC is used for producing the lipid particle is in one embodiment of from 40 to 85, in one embodiment of from 50 to 80, in one embodiment of from 54 to 75.

The number of DPPC molecules per apolipoprotein monomer in the lipid particle when pure DPPC is used for producing the lipid particle is in one embodiment of from 50 to 150, in one embodiment of from 65 to 135, in one embodiment of from 76 to 123, and in one embodiment of from 86 to 102.

The number of phospholipid molecules per apolipoprotein monomer in the lipid particle when a mixture of POPC and DPPC at a molar ratio of 1:3 is used for producing the lipid particle is in one embodiment of from about 50 to about 120, in one embodiment of from about 65 to about 105, and in one embodiment of from about 72 to about 96.

The number of lipid molecules per apolipoprotein monomer in the lipid particle when a mixture of POPC and DPPC at a molar ratio of 1:1 is used for producing the lipid particle is in one embodiment of from 50 to 120, in one embodiment of from 60 to 100, in one embodiment of from 71 to 92, and in one embodiment of from 71 to 85.

The number of lipid molecules per apolipoprotein monomer in the lipid particle when a mixture of POPC and DPPC at a molar ratio of 3:1 is used for producing the lipid particle is in one embodiment of from 50 to 105.

The number of lipid molecules per apolipoprotein monomer in the lipid particle when a mixture of POPC and DPPC at a molar ratio of 3:1 is used for producing the lipid particle is in one embodiment of from 60 to 95.

The number of lipid molecules per apolipoprotein monomer in the lipid particle when a mixture of POPC and DPPC at a molar ratio of 3:1 is used for producing the lipid particle is in one embodiment of from 60 to 90.

The number of lipid molecules per apolipoprotein monomer in the lipid particle when a mixture of POPC and DPPC at a molar ratio of 3:1 is used for producing the lipid particle is in one embodiment of from 60 to 88.

The number of lipid molecules per apolipoprotein monomer in the lipid particle when a mixture of POPC and DPPC at a molar ratio of 3:1 is used for producing the lipid particle is in one embodiment of from 62 to 80.

The number of lipid molecules per apolipoprotein monomer in the lipid particle when a mixture of POPC and DPPC at a molar ratio of 3:1 is used for producing the lipid particle is in one embodiment of from 66 to 86.

The number of lipid molecules per apolipoprotein monomer in the lipid particle when a mixture of POPC and DPPC at a molar ratio of 3:1 is used for producing the lipid particle is in one embodiment of from 64 to 70.

The number of lipid molecules per apolipoprotein monomer in the lipid particle when a mixture of POPC and DPPC at a molar ratio of 3:1 is used for producing the lipid particle is in one embodiment about 66.

For the production of a lipid particle comprising apolipoprotein and POPC a molar ratio of apolipoprotein to POPC in one embodiment of from 1:40 to 1:100 is employed, in one embodiment a molar ratio of from 1:40 to 1:80 is employed, and in one embodiment a molar ratio of about 1:60 is employed.

For the production of a lipid particle comprising apolipoprotein and DPPC a molar ratio of apolipoprotein to DPPC in one embodiment of from 1:70 to 1:100 is employed, in one embodiment a molar ratio of from 1:80 to 1:90 is employed, and in one embodiment a molar ratio of about 1:80 is employed.

For the production of a lipid particle comprising apolipoprotein, POPC and DPPC a molar ratio of apolipoprotein to POPC and DPPC with POPC and DPPC at a 1:3 molar ratio in one embodiment of from 1:60 to 1:100 is employed, in one embodiment a molar ratio of from 1:70 to 1:90 is employed, and in one embodiment a molar ratio of about 1:80 is employed.

For the production of a lipid particle comprising apolipoprotein, DPPC and POPC a molar ratio of apolipoprotein to POPC and DPPC with POPC and DPPC at a 1:1 molar ratio in one embodiment of from 1:60 to 1:100 is employed, in one embodiment a molar ratio of from 1:60 to 1:80 is employed, and in one embodiment a molar ratio of about 1:70 is employed.

For the production of a lipid particle comprising apolipoprotein, DPPC and POPC a molar ratio of apolipoprotein to POPC and DPPC with POPC and DPPC at a 3:1 molar ratio in one embodiment of from 1:60 to 1:100 is employed, in one embodiment a molar ratio of from 1:50 to 1:70 is employed, and in one embodiment a molar ratio of about 1:60 is employed.

In one embodiment if a mixture of lipids is used for producing the lipid particle the mixture has a phase transition temperature of from 4° C. to 45° C., in one embodiment of from 10° C. to 38° C., and in one embodiment of from 15° C. to 35° C.

For the formation of lipid particles comprising apolipoprotein different methods are known, such as freeze-drying, freeze-thawing, detergent solubilization followed by dialysis, microfluidization, sonification, and homogenization.

The lipid particle comprises in one embodiment an average number of from 1 to 10 apolipoprotein molecules per lipid particle, in one embodiment of from 1 to 8 apolipoprotein molecules per lipid particle, and in one embodiment of from 1 to 4 apolipoprotein molecules per lipid particle.

In one embodiment the lipid particle comprises an average number of at least 1, or 2, or 3, or 4, or 5, or 6, or 7, or 8, or 9, or 10 apolipoprotein molecules per lipid particle.

In one embodiment the lipid particle comprises one or more further polypeptides beside the apolipoprotein.

Without limitation the lipid particle may serve as an enzymatic co-factor and/or a carrier for taking up lipids, especially cholesterol.

Beside the synthetic detergent one or more detergents can be present in the lipid particle as reported herein. Such a detergent can be any detergent, i.e. a pharmaceutically acceptable detergent or other detergents at non-toxic concentrations, such as a non-ionic or ionic detergent. The non-ionic detergent can be an alkylene oxide derivative of an organic compound which contains one or more hydroxyl groups. In one embodiment the non-ionic detergent is selected from ethoxylated and/or propoxylated alcohol or ester compounds or mixtures thereof. In another embodiment the ester is selected from esters of sorbitol and fatty acids, such as sorbitan monooleate or sorbitan monopalmitate, oily sucrose esters, polyoxyethylene sorbitane fatty acid esters, polyoxyethylene sorbitol fatty acid esters, polyoxyethylene fatty acid esters, polyoxyethylene alkyl ethers, polyoxyethylene sterol ethers, polyoxyethylene-polypropoxy alkyl ethers, block polymers and cethyl ether, polyoxyethylene castor oil or hydrogenated castor oil derivatives and polyglycerine fatty acid esters. In one embodiment the non-ionic detergent is selected from Pluronic®, Poloxamer®, Span®, Tween®, Polysorbate®, Tyloxapol®, Emulphor® or Cremophor®.

The ionic detergent can be a bile duct agent. In one embodiment the ionic detergent is selected from cholic acid or deoxycholic acid, or their salts and derivatives, or from free fatty acids, such as oleic acid, linoleic acid and others.

In one embodiment the ionic detergent is selected from cationic lipids like C₁₀-C₂₄ alkylamine or alkanolamine and cationic cholesterol esters. In one embodiment the detergent is a detergent with a high CMC. In a further embodiment the detergent is a detergent with a CMC of at least 5 mM.

In one embodiment the lipid particle comprises less than 0.75% by weight detergent.

In one embodiment the lipid particle comprises less than 0.30% by weight detergent.

In one embodiment the lipid particle comprises less than 0.1% by weight detergent.

In one embodiment the lipid particle comprises less than 0.05% by weight detergent.

In one embodiment the detergent is selected from sugar-based detergents, polyoxyalkylene-based detergents, bile-salt based detergents, synthetic detergents or a combination thereof. In another embodiment the detergent is cholic acid or Zwittergent.

In one embodiment of the methods according to the invention the first solution is substantially free of lipid particles.

In one embodiment the method comprises after ii) and prior to iii) the following iia) incubating the solution obtained in ii). In one embodiment the incubating is for about 12 hours to about 20 hours. In another embodiment the incubating is for about 16 hours.

In one embodiment the incubating and/or removing is at a temperature of from 4° C. to 45° C.

In one embodiment the removing is by diafiltration or dialysis.

In one embodiment the first solution has a first volume, the second solution has a second volume, the polypeptide, such as an apolipoprotein, in the first solution has a defined concentration, the lipids and the detergent in the second solution each have a defined concentration, wherein in ii) the concentration of the apolipoprotein, of the lipids, and of the detergent is changed/reduced allowing the formation of a lipid particle.

With the dilution of the apolipoprotein solution and the addition of lipids and detergent suited ratios of apolipoprotein to lipids on the one hand and also suited ratios of the lipids to the detergent on the other hand are adjusted allowing the formation of a lipid particle.

In one embodiment the method comprises the following:
iv) purifying the lipid particle and thereby producing a lipid particle.

Different lipid particles can be formed with the method as reported herein.

For example for the production of lipid particle comprising an apolipoprotein saturated phospholipids containing carboxylic acid moieties with a chain of 16 atoms and shorter would be chosen from a technical point of view (e.g. dipalmitoyl-sn-glycero-3-phosphocholine, DPPC; dimyristoyl-sn-glycero-3-phosphocholine, DMPC etc.). In contrast thereto from biological data it can be assumed that non-saturated phospholipids containing carboxylic acid moieties with a chain of at least 16 C-atoms (e.g. palmitoyl-2-oleoyl-sn-glycero-3-phosphocholine, POPC; stearoyl-2-oleoyl-sn-glycero-3-phosphocholine, SOPC) are more effective and non-liver toxic.

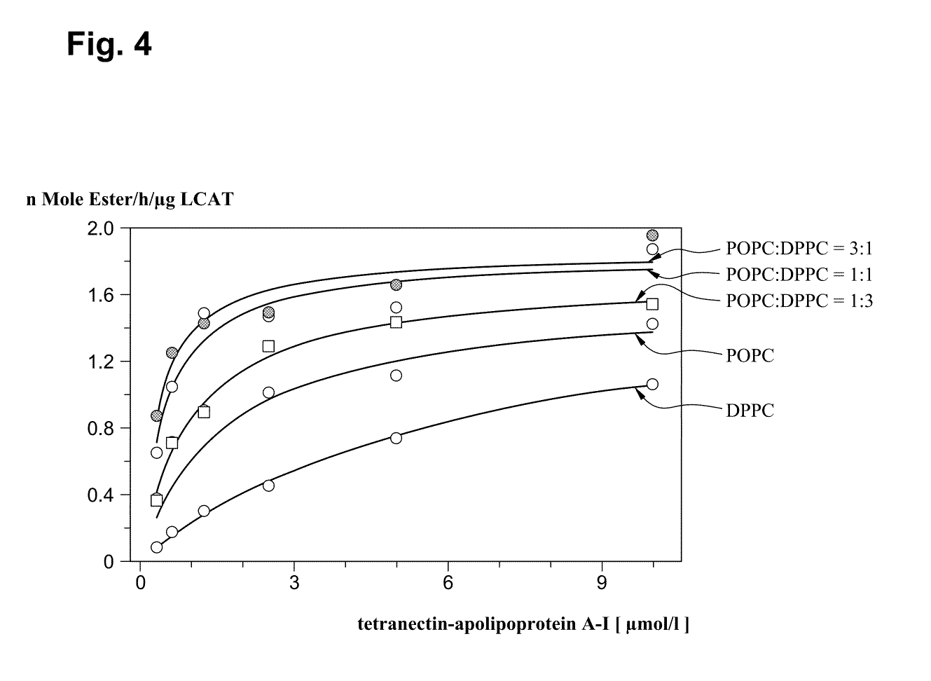

The phosphatidylcholines DPPC and POPC and mixtures thereof can be used for the formation of lipid particles containing an apolipoprotein. These exemplary phosphatidylcholines differ in one carboxylic acid moiety and have one identical carboxylic acid moiety esterified to the phosphoglycerol backbone. The manufacture of lipid particles was easier when DPPC was used. In contrast POPC was more effective in in vitro functional assays, particularly as substrate for the activation of the lecithin cholesterol acetyl transferase (LCAT) enzyme which is necessary for the conversion of the mobilized cholesterol into cholesterol ester. It has been found that lipid particles comprising mixtures of two phosphatidylcholines, as e.g. POPC and DPPC, in different molar ratios have improved properties compared to lipid particles comprising only one phosphatidylcholine (see e.g. FIG. 4).

For example the lipid particle can contain only POPC. The number of POPC molecules per apolipoprotein monomer may vary between 54 and 75 when molar ratios from 1:40 up to 1:80 of apolipoprotein to lipid are used in the production of the lipid particle. In one embodiment the molar ratio of apolipoprotein to POPC is of from 1:40 to 1:80, in one embodiment the molar ratio is of from 1:50 to 1:70, in one embodiment the molar ratio is about 1:60.

Thus, for the production of a lipid particle comprising apolipoprotein and POPC the molar ratio of apolipoprotein to POPC is in one embodiment of from 1:40 to 1:100, in one embodiment the molar ratio is of from 1:40 to 1:80, and in one embodiment the molar ratio is about 1:60.

For example the lipid particle can contain only DPPC. The number of DPPC molecules per apolipoprotein monomer may vary between 76 and 123 when molar ratios from 1:40 up to 1:80 of apolipoprotein to lipid are used in the production of the lipid particle. In one embodiment the molar ratio of apolipoprotein to DPPC is of from 1:70 to 1:100, in one embodiment the molar ratio is of from 1:75 to 1:90, in one embodiment the molar ratio is about 1:80.

For example the lipid particle can be produced starting from a mixture of POPC and DPPC at a molar ratio of 1:3. The number of phospholipid molecules per apolipoprotein monomer may vary between 72 and 112 when molar ratios from 1:60 up to 1:100 of apolipoprotein to lipid are used in the production of the lipid particle. In one embodiment the molar ratio of apolipoprotein to POPC and DPPC is of from 1:70 to 1:90, in one embodiment the molar ratio is of from 1:75 to 1:85, in one embodiment the molar ratio is about 1:80.

Thus, for the production of a lipid particle comprising apolipoprotein, POPC and DPPC the molar ratio of apolipoprotein to POPC and DPPC with POPC and DPPC at a 1:3 molar ratio is in one embodiment of from 1:60 to 1:100, in one embodiment the molar ratio is of from 1:70 to 1:90, and in one embodiment the molar ratio is about 1:80.

For example the lipid particle can be produced starting from a mixture of POPC and DPPC at a molar ratio of 1:1. The number of phospholipid molecules per apolipoprotein monomer may vary between 71 and 111 when molar ratios from 1:60 up to 1:100 of apolipoprotein to lipid are used in the production of the lipid particle. In one embodiment the molar ratio of apolipoprotein to POPC and DPPC is of from 1:60 to 1:80, in one embodiment the molar ratio is of from 1:65 to 1:75, in one embodiment the molar ratio is about 1:70.

Thus, for the production of a lipid particle comprising apolipoprotein, DPPC and POPC the molar ratio of apolipoprotein to POPC and DPPC with POPC and DPPC at a 1:1 molar ratio is in one embodiment of from 1:60 to 1:100, in one embodiment the molar ratio is of from 1:60 to 1:80, and in one embodiment the molar ratio is about 1:70.

For example the lipid particle can be produced starting from a mixture of POPC and DPPC at a molar ratio of 3:1. The number of phospholipid molecules per apolipoprotein monomer can vary between 46 and 93 when molar ratios from 1:60 up to 1:100 of apolipoprotein to lipid are used in the production of the lipid particle. In one embodiment the molar ratio of apolipoprotein to POPC and DPPC is of from 1:50 to 1:70, in one embodiment the molar ratio is of from 1:55 to 1:65, in one embodiment the molar ratio is about 1:60.

Thus, for the production of a lipid particle, which comprises apolipoprotein, DPPC and POPC, the molar ratio of apolipoprotein to POPC and DPPC, whereby POPC and DPPC are at a 3:1 molar ratio, is in one embodiment of from 1:60 to 1:100, in one embodiment the molar ratio is of from 1:50 to 1:70, and in one embodiment the molar ratio is about 1:60.

In one embodiment the apolipoprotein is provided as an aqueous solution of the apolipoprotein and can be obtained from downstream processing after recombinant production or any other source of apolipoprotein production and can comprise different concentrations of apolipoprotein with varying purity.

Basically lipid particle formation is achieved by incubating a polypeptide with detergent solubilized lipids at their respective transition temperature. Removal of the detergent by dialysis results in the formation of lipid particles consisting of a lipid bilayer.

For example, lipid particle formation is achieved by incubating tetranectin-apolipoprotein A-I or a multimer thereof with detergent solubilized lipids at their respective transition temperature. Removal of the detergent by dialysis results in the formation of lipid particles consisting of a lipid bilayer surrounded by the α-helical apolipoprotein.

The lipid particle can be purified by a combination of precipitation and/or chromatography steps. For example excess detergent, i.e. detergent not part of the lipid particle, can be removed in a hydrophobic adsorption chromatography step. In one embodiment a step of the method for purifying a lipid particle comprises a hydrophobic adsorption chromatography step. In another embodiment the chromatographic material for the hydrophobic adsorption step is selected from Extracti Gel D (available from Pierce Biotechnology, Rockford Ill., USA), CALBIOSORB™ (available from Calbiochem, San Diego, Calif., USA), SDR 30 HyperD™ Solvent-Detergent Removal Chromatography Resin (available from PALL Corporation, Ann Arbor, Mich., USA). The lipid particle is recovered from the hydrophobic adsorption material with a detergent-free solution. This chromatography step is especially useful for detergents with a low CMC.

In one embodiment dialysis is used to remove a detergent with a high CMC.

### Pharmaceutical and Diagnostic Composition:

The lipid particle obtained by a method as reported herein can be used for the treatment and/or diagnosis of a disease or condition.

The tetranectin-apolipoprotein A-I as reported herein or the lipid particle as reported herein can be used for the treatment and/or diagnosis of a disease or condition characterized by non-normal lipid levels or a deposition of lipid within body components, such as plaques in blood vessels.

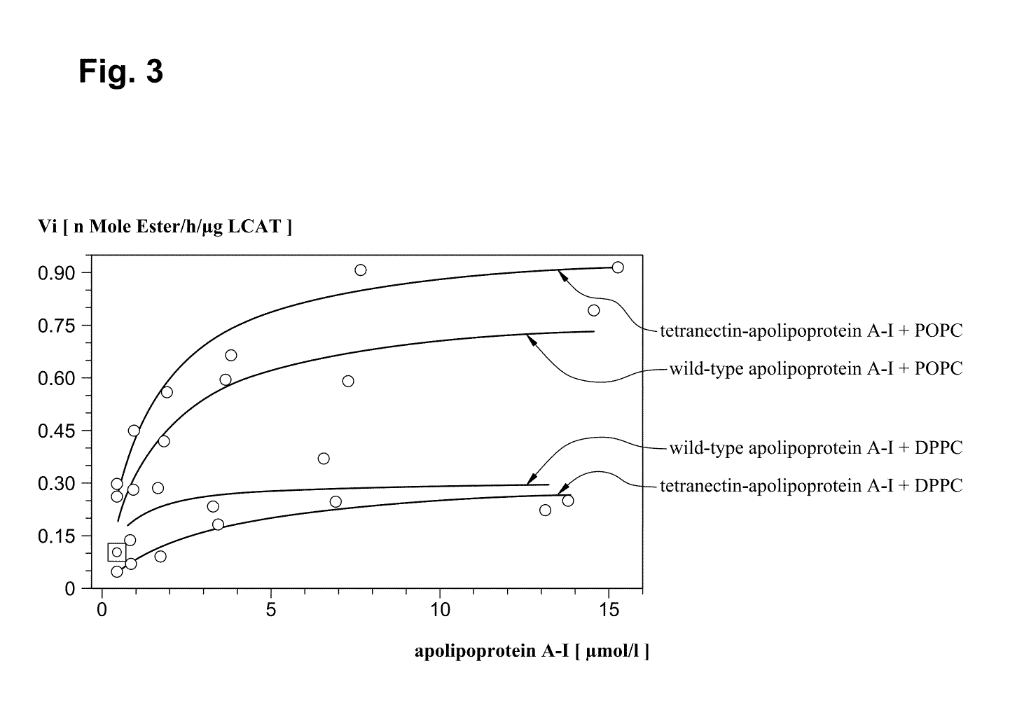

In order to determine the capacity of the resulting protein-lipid complex to support LCAT catalyzed cholesterol esterification cholesterol was incorporated in the lipid particle as reported herein by quick addition of an ethanolic cholesterol solution. Lipid particles containing pure POPC are better LCAT substrates than complexes containing DPPC independent of their apolipoprotein constituent, such as wild-type apolipoprotein A-I or tetranectin-apolipoprotein A-I (FIG. 3).

Initial velocity of cholesterol esterification in lipid particles comprising different mixtures of POPC and DPPC shows that mixtures are better LCAT substrates than any of the pure phosphatidylcholine as can be seen from the initial velocities of cholesterol esterification (see Table 3 and FIG. 4).

TABLE 3
Initial velocities of cholesterol esterification in lipid
particles comprising different mixtures of phospholipids.

| phospholipid | | |
|---|---|---|
| molar ratio used | | |
| for producing the | Kₘ | Vₘₐₓ |
| lipid particle | [μm] | [nmol ester/h/μg LCAT] |
| POPC | 4.6 | 1.6 |
| POPC:DPPC 3:1 | 0.4 | 1.9 |
| POPC:DPPC 1:1 | 0.5 | 1.8 |
| POPC:DPPC 1:3 | 1.0 | 1.7 |
| DPPC | 0.9 | 1.8 |

Macrophage like human THP1 cells obtained by exposing THP-1 monocytic leukemia cells to phorbol myristate acetate and loaded with a radioactive labeled cholesterol tracer were exposed to cholesterol acceptor test compounds.

Efflux velocity induced by acceptor test compounds can be calculated as the ratio of cholesterol radioactivity in the supernatant to the sum of the radioactivity in the cells plus their supernatant and compared to cells exposed to medium containing no acceptors and analyzed by linear fit. Parallel experiments can be performed using cells exposed and not exposed to a RXR-LXR agonist which is known to upregulate mainly ABCA-1 and bias efflux toward ABCA-1 mediated transport.

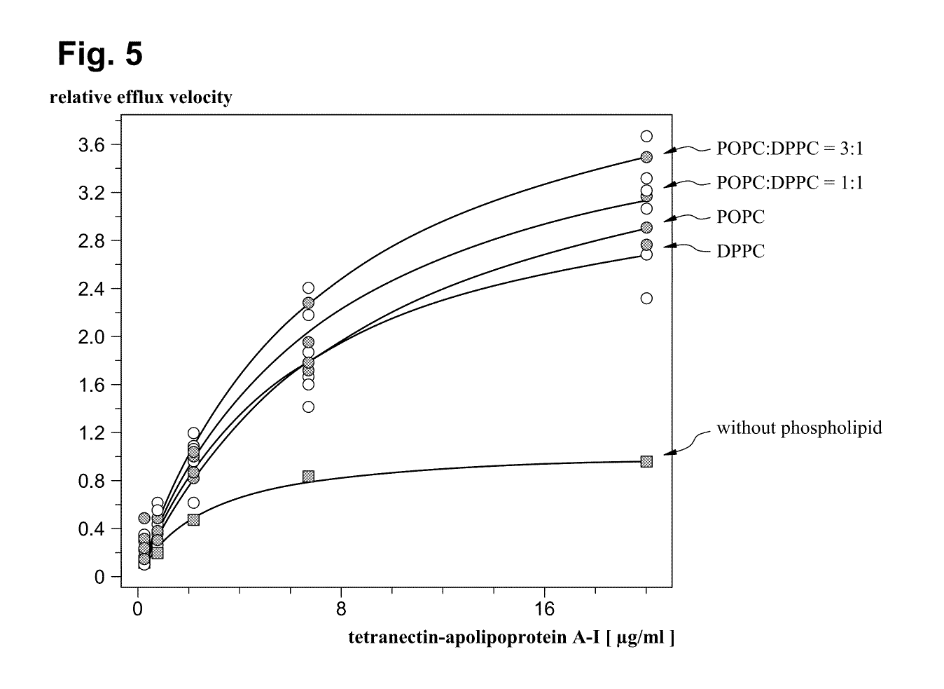

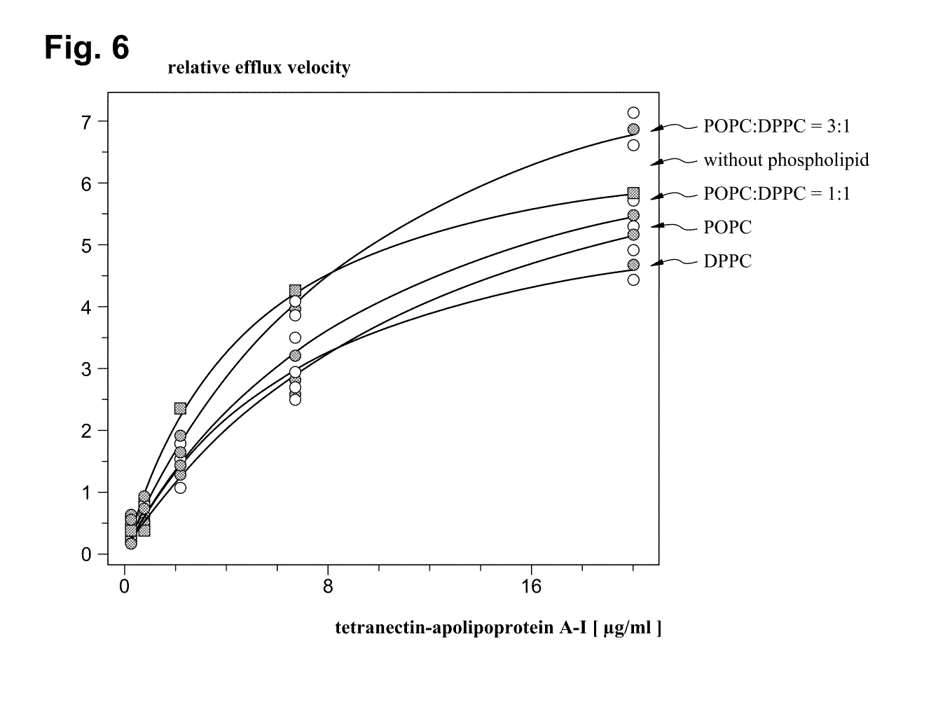

In cells not pre-treated with RXR-LXR lipid particles a higher increase in cholesterol efflux compared to the efflux obtained with non lipidated tetranectin-apolipoprotein A-I can be seen. Only a small influence of the lipid mixture on efflux can be observed in the tested series (FIG. 5). In cells pre-treated with RXR-LXR a comparable increase in cholesterol efflux of lipid particles a non-lipidated tetranectin-apolipoprotein A-I can be seen. The overall increase was higher compared to that observed with not pre-treated cells. Only a small influence of the lipid mixture on efflux can be observed in the tested series (FIG. 6).

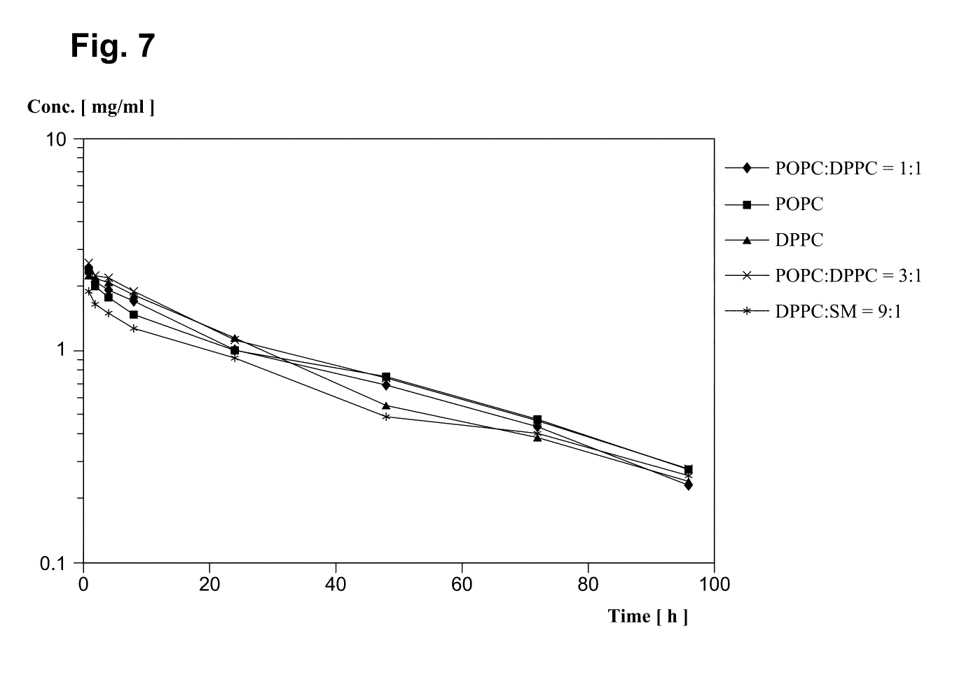

Different lipid particles were tested in vivo in rabbits. The lipid particle was applied as intravenous infusion and serial blood sampling was performed over 96 h after application. Values of liver enzymes, cholesterol, and cholesterol ester were determined. Plasma concentrations are comparable for all tested lipid particles comprising an initial distribution phase followed by log-linear decline of plasma concentrations (FIG. 7). As can be seen from Table 4 pharmacokinetic parameters are similar for all tested compounds. The observed half-lives are close to 1.5 days.

TABLE 4
Determined pharmacokinetic parameters.

| phospholipid | | | | |
|---|---|---|---|---|
| molar ratio used | | | | |
| for producing the | C_{L} | vₛₛ | T_{1/2} | Cₘₐₓ |
| lipid particle | [ml/h/kg] | [ml/kg] | [h] | [mg/ml] |
| POPC | 0.89 ± 0.22 | 45.0 ± 2.5 | 36.9 ± 8.2 | 2.40 ± 0.19 |
| POPC:DPPC 3:1 | 0.82 ± 0.06 | 37.8 ± 5.6 | 34.2 ± 4.5 | 2.65 ± 0.28 |
| POPC:DPPC 1:1 | 0.85 ± 0.14 | 43.1 ± 5.9 | 38.6 ± 10.6 | 2.34 ± 0.31 |
| DPPC | 0.96 ± 0.10 | 37.8 ± 4.9 | 30.2 ± 7.7 | 2.29 ± 0.19 |
| DPPC:SM 9:1 | 1.28 ± 0.62 | 50.7 ± 8.7 | 31.3 ± 8.2 | 1.91 ± 0.33 |

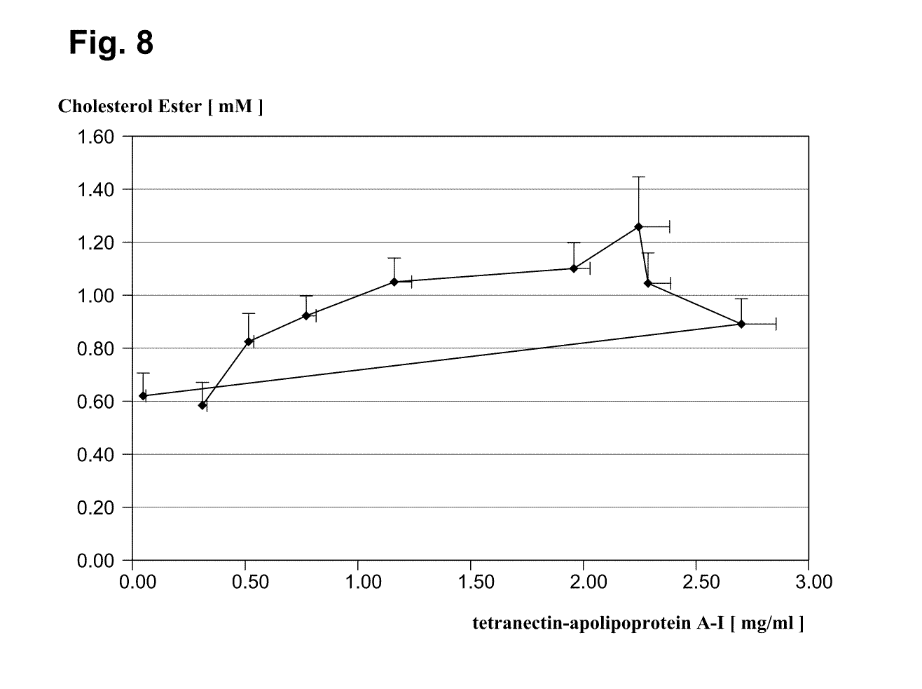

As can be seen from FIG. 8 cholesterol is mobilized and esterified in plasma. Plasma cholesterol ester levels do continue to increase even after the concentration of tetranectin-apolipoprotein A-I is already decreasing. When plasma tetranectin-apolipoprotein A-I levels have decreased to about 0.5 mg/ml (about 50% of normal wild-type apolipoprotein A-I) increased cholesterol ester levels can still be detected.

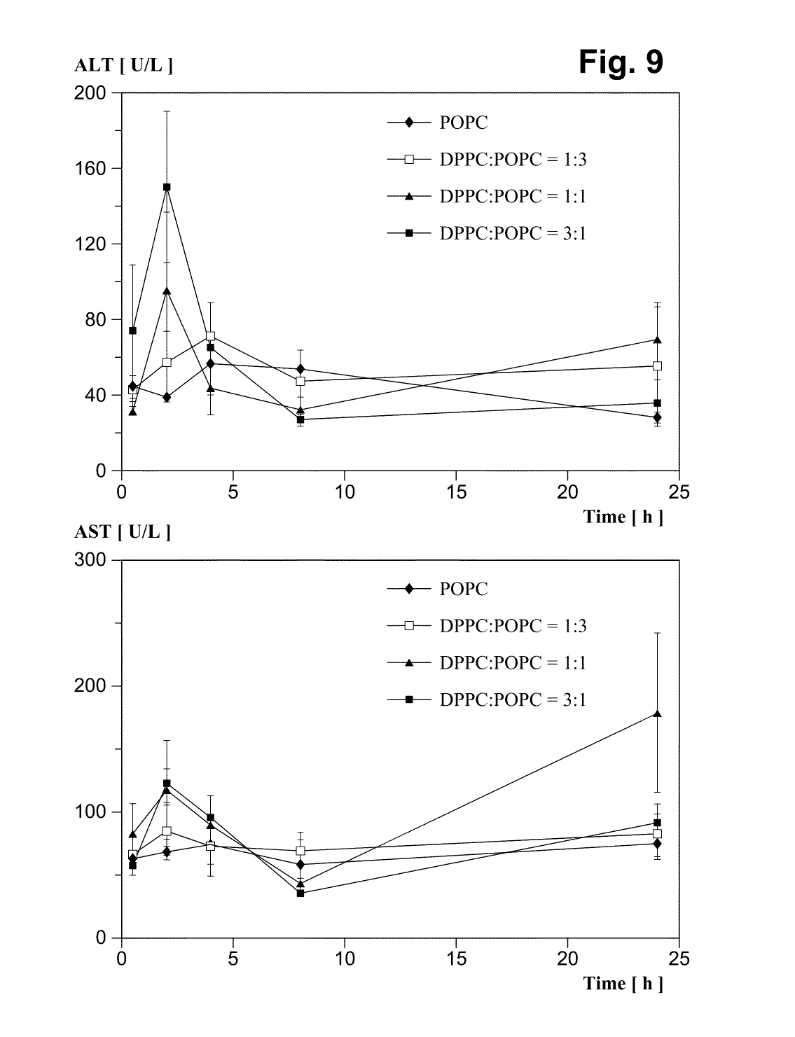

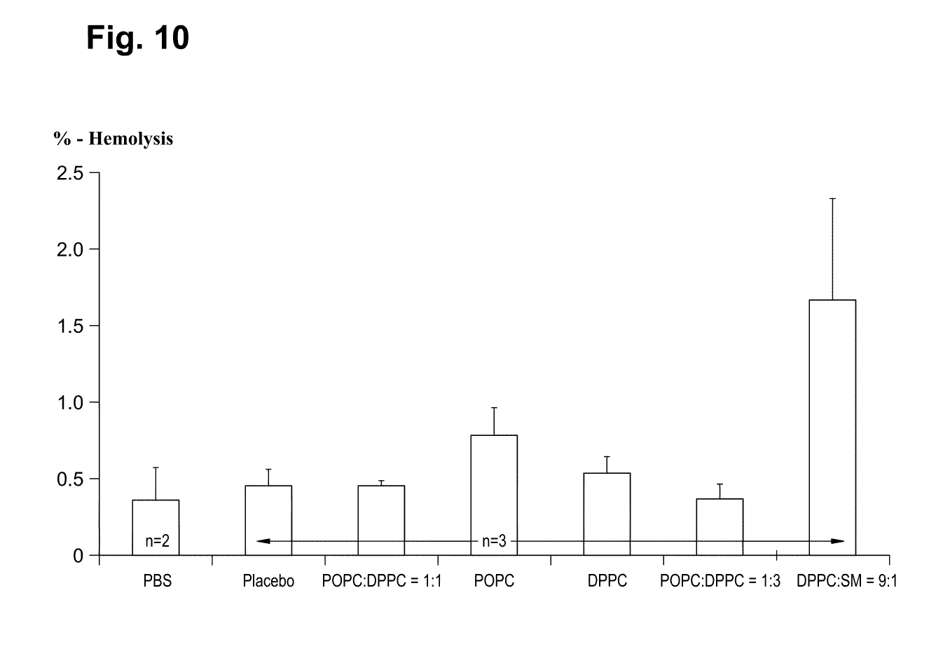

Lipid particles comprising tetranectin-apolipoprotein A-I do not induced liver enzymes in rabbits as well as in mice as can be seen from FIGS. 1 and 9. Also no hemolysis can be determined in plasma samples obtained two hours after intravenous application (FIG. 10).

Therefore aspects of the current invention are a pharmaceutical composition and a diagnostic composition comprising a lipid particle comprising apolipoprotein as reported herein or a tetranectin-apolipoprotein A-I as reported herein.

The lipid particle as reported herein has improved in vivo properties compared to non-lipidated apolipoprotein and other lipid particles as shown in the following Table 5.

TABLE 5
In vivo properties of different apolipoproteins and lipid particles.

| | lipid | | highest | acute liver | |
|---|---|---|---|---|---|
| | particle | | applied | toxicological | |
| protein | comprising | applied to | dose | effect | reference |
| apolipoprotein | no particle | rat | orally, 1 g/kg | no toxic | US 2005/0287636 |
| A-I mutants | | | | effect up to | |
| | | | | 500 mg/kg | |
| A-I, | DMPC | mouse | i.v. 1 to 1.2 | not described | WO 2002/38609; |
| tetranectin- | | | mg/mouse | | Graversen (2008) |
| apolipoprotein | | | | | |
| A-I | | | | | |
| pro | SM | not reported | not reported | injection, | WO 2003/096983 |
| apolipoprotein | | | | toxic at dose | |
| A-I | | | | of 200 mg/kg | |
| apolipoprotein | PG/SM | rabbit | i.v. 15 mg/kg | not described | WO 2006/100567 |
| A-I | | | | | |
| apolipoprotein | PC | human | 80 mg/kg | treatment group was | WO 2007/137400 |
| A-I | (soybean) | | | discontinued early | |
| | | | | because of liver | |
| | | | | function test | |
| | | | | abnormalities | |
| | | | | (10-fold increase | |
| | | | | in alanine | |
| | | | | aminotransferase) | |
| apolipoprotein | POPC | human | 45 mg/kg | one patient withdrawn | Nissen, S. E., et |
| A-I Milano | | | | due to development | al, JAMA 290 |
| variant | | | | of an elevated | (2003) 2292-2300 |
| | | | | aspartate amino- | |
| | | | | transferase level (3x upper | |
| | | | | limit of normal) | |
| tetranectin- | DMPC | rabbit | 100 mg/kg | lethal after 3-4 | |
| apolipoprotein | | | | hours in all | |
| A-I | | | | animals tested | |
| tetranectin- | POPC/ | rabbit | 100 mg/kg | increase not | |
| apolipoprotein | DPPC | | | observed | |
| A-I | | | | | |
| tetranectin- | POPC/ | rat | i.v. 500 mg/kg | increase not | |
| apolipoprotein | DPPC | | | observed | |
| A-I | | | | | |
| tetranectin- | POPC/ | cynomolgus | i.v. 200 mg/kg | increase not | |
| apolipoprotein | DPPC | monkey | | observed | |
| A-I | | | | | |

The efficiency at which cholesterol is mobilized into the blood can be shown by monitoring the ratio of cholesterol concentration in the blood to apolipoprotein concentration in the blood, especially when the ratio of the AUC values (area under the curve) of these parameters determined in vivo time dependent after application is taken.

The lipid particle as reported herein, especially a lipid particle comprising a tetranectin-apolipoprotein of SEQ ID NO: 01 and POPC and DPPC at a molar ratio of 3:1, shows enhanced cholesterol mobilization in vivo.

### Tetranectin-Apolipoprotein A-I

Beside the lipid particle as outlined above is herein reported also a tetranectin-apolipoprotein A-I.

Tetranectin-apolipoprotein A-I is a fusion protein of the human tetranectin trimerising structural element and the wild-type human apolipoprotein A-I. The amino acid sequence of the human tetranectin part can be shortened by the first 9 amino acids starting with the isoleucine residue of position 10, a naturally occurring truncation site. As a consequence of this truncation the 0-glycosylation site at threonine residue of position 4 has been deleted. Between the tetranectin trimerising structural element and the human apolipoprotein A-I the five amino acid residues “SLKGS” (SEQ ID NO: 03) were removed.

For improved expression and purification a construct can be generated comprising an N-terminal purification tag, e.g. a hexahistidine-tag, and an IgA protease cleavage site. As a result of the specific cleavage two amino acids—as first alanine or glycine or serine or proline and as second proline—are maintained at the N-terminus of the tetranectin-apolipoprotein A-I. The tetranectin-apolipoprotein A-I can have the amino acid sequence of SEQ ID NO: 01.

The tetranectin trimerising structural element provides for a domain that allows for the formation of a trimeric tetranectin-apolipoprotein A-I multimer that is constituted by non-covalent interactions between each of the individual tetranectin-apolipoprotein A-I monomers.

By using an alternative purification method, the purification-tag and the IgA protease cleavage site can be omitted resulting in a tetranectin-apolipoprotein A-I of the amino acid sequence of SEQ ID NO: 02.

In one embodiment the apolipoprotein can be a variant comprising conservative amino acid substitutions or an apolipoprotein A-I mimic.

Apolipoprotein A-I can be determined enzymatically, via NMR spectroscopy, or by using monoclonal or polyclonal anti-apolipoprotein-A-I antibodies. Other aspects as reported herein are therefore polyclonal and monoclonal antibodies specifically binding the tetranectin-apolipoprotein A-I as reported herein. Such antibodies can be obtained with methods known to a person skilled in the art. Also the labeling of the antibodies for use in immunoassays can be performed with methods known to a person of skill in the art.

In one embodiment the apolipoprotein can be a variant comprising conservative amino acid substitutions, or an apolipoprotein A-I mimic. In one embodiment the tetranectin-apolipoprotein A-I has the amino acid sequence of SEQ ID NO: 02, or SEQ ID NO: 66, or SEQ ID NO: 67, wherein X is selected from SEQ ID NO: 68 to SEQ ID NO: 105.

Thus, in one embodiment the tetranectin-apolipoprotein A-I has the amino acid sequence of

(SEQ ID NO: 02)
IVNAKKDVVNTKMFEELKSRLDTLAQEVALLKEQQALQTVDEPPQSPWD
RVKDLATVYVDVLKDSGRDYVSQFEGSALGKQLNLKLLDNWDSVTSTFS
KLREQLGPVTQEFWDNLEKETEGLRQEMSKDLEEVKAKVQPYLDDFQKK
WQEEMELYRQKVEPLRAELQEGARQKLHELQEKLSPLGEEMRDRARAHV
DALRTHLAPYSDELRQRLAARLEALKENGGARLAEYHAKATEHLSTLSE
KAKPALEDLRQGLLPVLESFKVSFLSALEEYTKKLNTQ.

In one embodiment the tetranectin-apolipoprotein A-I has the amino acid sequence of

(SEQ ID NO: 66)
(A, G, S, T)PIVNAKKDVVNTKMFEELKSRLDTLAQEVALLKEQQA
LQTVDEPPQSPWDRVKDLATVYVDVLKDSGRDYVSQFEGSALGKQLNLK
LLDNWDSVTSTFSKLREQLGPVTQEFWDNLEKETEGLRQEMSKDLEEVK
AKVQPYLDDFQKKWQEEMELYRQKVEPLRAELQEGARQKLHELQEKLSP
LGEEMRDRARAHVDALRTHLAPYSDELRQRLAARLEALKENGGARLAEY
HAKATEHLSTLSEKAKPALEDLRQGLLPVLESFKVSFLSALEEYTKKLN
TQ.

In one embodiment the tetranectin-apolipoprotein A-I has the amino acid sequence of

(SEQ ID NO: 67)
(M)HHHHHHXIVNAKKDVVNTKMFEELKSRLDTLAQEVALLKEQQAL
QTVDEPPQSPWDRVKDLATVYVDVLKDSGRDYVSQFEGSALGKQLNL
KLLDNWDSVTSTFSKLREQLGPVTQEFWDNLEKETEGLRQEMSKDLE
EVKAKVQPYLDDFQKKWQEEMELYRQKVEPLRAELQEGARQKLHELQ
EKLSPLGEEMRDRARAHVDALRTHLAPYSDELRQRLAARLEALKENG
GARLAEYHAKATEHLSTLSEKAKPALEDLRQGLLPVLESFKVSFLSA
LEEYTKKLNTQ,

wherein X can be any of the following amino acid sequences

(SEQ ID NO: 68)
A, G, S, P, AP, GP, SP, PP, GSAP,
(SEQ ID NO: 69)
GSGP,
(SEQ ID NO: 70)
GSSP,
(SEQ ID NO: 71)
GSPP,
(SEQ ID NO: 72)
GGGS,
(SEQ ID NO: 73)
GGGGS,
(SEQ ID NO: 74)
GGGSGGGS,
(SEQ ID NO: 75)
GGGGSGGGGS,
(SEQ ID NO: 76)
GGGSGGGSGGGS,
(SEQ ID NO: 77)
GGGGSGGGGSGGGGS,
(SEQ ID NO: 78)
GGGSAP,
(SEQ ID NO: 79)
GGGSGP,
(SEQ ID NO: 80)
GGGSSP,
(SEQ ID NO: 81)
GGGSPP,
(SEQ ID NO: 82)
GGGGSAP,
(SEQ ID NO: 83)
GGGGSGP,
(SEQ ID NO: 84)
GGGGSSP,
(SEQ ID NO: 85)
GGGGSPP,
(SEQ ID NO: 86)
GGGSGGGSAP,
(SEQ ID NO: 87)
GGGSGGGSGP,
(SEQ ID NO: 88)
GGGSGGGSSP,
(SEQ ID NO: 89)
GGGSGGGSPP,
(SEQ ID NO: 90)
GGGSGGGSGGGSAP,
(SEQ ID NO: 91)
GGGSGGGSGGGSGP,
(SEQ ID NO: 92)
GGGSGGGSGGGSSP,
(SEQ ID NO: 93)
GGGSGGGSGGGSPP,
(SEQ ID NO: 94)
GGGGSAP,
(SEQ ID NO: 95)
GGGGSGP,
(SEQ ID NO: 96)
GGGGSSP,
(SEQ ID NO: 97)
GGGGSPP,
(SEQ ID NO: 98)
GGGGSGGGGSAP,
(SEQ ID NO: 99)
GGGGSGGGGSGP,
(SEQ ID NO: 100)
GGGGSGGGGSSP,
(SEQ ID NO: 101)
GGGGSGGGGSPP,
(SEQ ID NO: 102)
GGGGSGGGGSGGGGSAP,
(SEQ ID NO: 103)
GGGGSGGGGSGGGGSGP,
(SEQ ID NO: 104)
GGGGSGGGGSGGGGSSP,
and
(SEQ ID NO: 105)
GGGGSGGGGSGGGGSPP.

If a heterologous polypeptide is produced in E. coli strains the amino-terminal methionine residue is usually not efficiently cleaved off by proteases, thus the amino-terminal methionine residue is partially present in the produced polypeptide.

The following examples are provided to aid the understanding of the present invention, the true scope of which is set forth in the appended claims. It is understood that modifications can be made in the procedures set forth without departing from the spirit of the invention.

### Materials and Methods

### Size-Exclusion-HPLC:

The chromatography was conducted with a Tosoh Haas TSK 3000 SWXL column on an ASI-100 HPLC system (Dionex, Idstein, Germany). The elution peaks were monitored at 280 nm by a UV diode array detector (Dionex). After dissolution of the concentrated samples to 1 mg/ml the column was washed with a buffer consisting of 200 mM potassium dihydrogen phosphate and 250 mM potassium chloride pH 7.0 until a stable baseline was achieved. The analyzing runs were performed under isocratic conditions using a flow rate of 0.5 ml/min. over 30 minutes at room temperature. The chromatograms were integrated manually with Chromeleon (Dionex, Idstein, Germany). Aggregation in % was determined by comparing the area under the curve (AUC) of high molecular weight forms with the AUC of the monomer peak.

### Dynamic Light Scattering (DLS):

DLS is a non-invasive technique for measuring particle size, typically in the sub-micron size range. In the current invention the Zetasizer Nano S apparatus (Malvern Instruments, Worcestershire, UK) with a temperature controlled quartz cuvette (25° C.) was used for monitoring a size range between 1 nm and 6 μm. The intensity of the back scattered laser light was detected at an angle of 173°. The intensity fluctuates at a rate that is dependent upon the particle diffusion speed, which in turn is governed by particle size. Particle size data can therefore be generated from an analysis of the fluctuation in scattered light intensity (Dahneke, B. E. (ed.), Measurement of Suspended Particles by Quasielectric Light Scattering, Wiley Inc. (1983); Pecora, R., Dynamic Light Scattering: Application of Photon Correlation Spectroscopy, Plenum Press (1985)). The size distribution by intensity was calculated using the multiple narrow mode of the DTS software (Malvern). Experiments were conducted with undiluted samples.

### SEC-MALLS:

SEC-MALLS is a combination of size exclusion chromatography with a three detector system: i) UV detection, ii) refraction index detection and iii) light scattering detection. For the separation by size a Superose 6 column 10/300 GL column from GE Healthcare is used. The method is run isocratically with a PBS buffer pH 7.4 applying a flow rate of 0.4 ml/min. Three detector systems are connected in series. The complete lipid particle (protein-lipid particle) signal is monitored by the refraction index detector whereas the UV absorbance determined at 280 nm determines the signal induced by the protein part. The proportion of the lipid fraction is obtained by a simple subtraction of the protein UV signal from the complete signal. Applying light scattering allows for the detection of the molecular mass of the respective species and, thus, a complete and detailed description of the lipid particle.

### Detergent Determination:

The determination of residual detergent was conducted by reversed-phase chromatography coupled with an evaporative light scattering detector (RP-ELSD). As column a Luna C18 4.6×150 mm, 5 μm, 100 Å from Phenomenex (Aschaffenburg, Germany) was used. After centrifugation through a 10 kDa membrane 90 μl of the flow-through were used for HPLC separation. Elution was performed under isocratic conditions with 74% (v/v) methanol solution containing 0.1% (v/v) trifluoro acetic acid. Column temperature was set to 30° C. Detection was performed by an evaporative light scattering detector applying a nebulization temperature of 30° C., an evaporating temperature of 80° C. and a gas flow of 1.0 l/min. Quantification of the residual detergent was conducted by the establishment of a calibration curve, in case of cholate in the range of 0.22 μg to 7.5 μg cholate.

### Protein Determination:

The protein concentration was determined by determining the optical density (OD) at 280 nm, using the molar extinction coefficient calculated on the basis of the amino acid sequence.

### Recombinant DNA Technique:

Standard methods were used to manipulate DNA as described in Sambrook, J., et al., Molecular cloning: A laboratory manual; Cold Spring Harbor Laboratory Press, Cold Spring Harbor, N.Y., 1989. The molecular biological reagents were used according to the manufacturer's instructions.

### Example 1

### Making and Description of the E. coli Expression Plasmids

The tetranectin-apolipoprotein A-I fusion polypeptide was prepared by recombinant means. The amino acid sequence of the expressed fusion polypeptide in N- to C-terminal direction is as follows:
the amino acid methionine (M),
a fragment of an interferon sequence that has the amino acid sequence of CDLPQTHSL (SEQ ID NO: 55),
a GS linker,
a hexa-histidine tag that has the amino acid sequence of HHHHHH (SEQ ID NO: 56),
a GS linker,
an IgA protease cleavage site that has the amino acid sequence of VVAPPAP (SEQ ID NO: 60), and
a tetranectin-apolipoprotein A-I that has the amino acid sequence of SEQ ID NO: 02.

The tetranectin-apolipoprotein A-I fusion polypeptides as described above are precursor polypeptides from which the tetranectin-apolipoprotein A-I fusion polypeptides was released by enzymatic cleavage in vitro using IgA protease.

The precursor polypeptide encoding fusion gene was assembled with known recombinant methods and techniques by connection of appropriate nucleic acid segments. Nucleic acid sequences made by chemical synthesis were verified by DNA sequencing. The expression plasmid for the production of tetranectin-apolipoprotein A-I of SEQ ID NO: 01 encoding a fusion protein of SEQ ID NO: 31 was prepared as follows.

Making of the E. coli Expression Plasmid

Plasmid 4980 (4980-pBRori-URA3-LACI-SAC) is an expression plasmid for the expression of core-streptavidin in E. coli. It was generated by ligation of the 3142 bp long EcoRI/CelII-vector fragment derived from plasmid 1966 (1966-pBRori-URA3-LACI-T-repeat; reported in EP-B 1 422 237) with a 435 bp long core-streptavidin encoding EcoRI/CelII-fragment.

The core-streptavidin E. coli expression plasmid comprises the following elements:
the origin of replication from the vector pBR322 for replication in E. coli (corresponding to by position 2517-3160 according to Sutcliffe, G., et al., Quant. Biol. 43 (1979) 77-90),
the URA3 gene of Saccharomyces cerevisiae coding for orotidine 5′-phosphate decarboxylase (Rose, M. et al. Gene 29 (1984) 113-124) which allows plasmid selection by complementation of E. coli pyrF mutant strains (uracil auxotrophy),
the core-streptavidin expression cassette comprising
   the T5 hybrid promoter (T5-PN25/03/04 hybrid promoter according to Bujard, H., et al. Methods. Enzymol. 155 (1987) 416-433 and Stueber, D., et al., Immunol. Methods IV (1990) 121-152) including a synthetic ribosomal binding site according to Stueber, D., et al. (see before),
   the core-streptavidin gene,
   two bacteriophage-derived transcription terminators, the λ-T0 terminator (Schwarz, E., et al., Nature 272 (1978) 410-414) and the fd-terminator (Beck E. and Zink, B. Gene 1-3 (1981) 35-58),
the lacI repressor gene from E. coli (Farabaugh, P. J., Nature 274 (1978) 765-769).

The final expression plasmid for the expression of the tetranectin-apolipoprotein A-I precursor polypeptide was prepared by excising the core-streptavidin structural gene from vector 4980 using the singular flanking EcoRI and CelII restriction endonuclease cleavage site and inserting the EcoRII/CelII restriction site flanked nucleic acid encoding the precursor polypeptide into the 3142 bp long EcoRI/CelII-4980 vector fragment.

### Example 2

### Expression of Tetranectin-Apolipoprotein A-I

For the expression of the fusion protein there was employed an E. coli host/vector system which enables an antibiotic-free plasmid selection by complementation of an E. coli auxotrophy (PyrF) (EP 0972838 and U.S. Pat. No. 6,291,245).

The E. coli K12 strain CSPZ-2 (leuB, proC, trpE, th-1, ΔpyrF) was transformed by electroporation with the expression plasmid p(IFN-His6-IgA-tetranectin-apolipoprotein A-I). The transformed E. coli cells were first grown at 37° C. on agar plates.

### Fermentation Protocol 1:

For pre-fermentation a M9 medium according to Sambrook et al (Molecular Cloning: A laboratory manual. Cold Spring Harbor Laboratory Press; 2nd edition (December 1989) supplemented with about 1 g/l L-leucine, about 1 g/l L-proline and about 1 mg/l thiamine-HCl has been used.

For pre-fermentation 300 ml of M9-medium in a 1000 ml Erlenmeyer-flask with baffles was inoculated with 2 ml out of a primary seed bank ampoule. The cultivation was performed on a rotary shaker for 13 hours at 37° C. until an optical density (578 nm) of 1-3 was obtained.

For fermentation a batch medium according to Riesenberg et al. was used (Riesenberg, D., et al., J. Biotechnol. 20 (1991) 17-27): 27.6 g/l glucose*H₂O, 13.3 g/l KH₂PO₄, 4.0 g/l (NH₄)₂HPO₄, 1.7 g/l citrate, 1.2 g/l MgSO₄*7H₂O, 60 mg/l iron(III)citrate, 2.5 mg/l CoCl₂*6H₂O, 15 mg/l MnCl₂*4H₂O, 1.5 mg/l CuCl₂*2H₂O, 3 mg/l H₃BO₃, 2.5 mg/l Na₂MoO₄*2H₂O, 8 mg/l Zn(CH₃COO)₂*2H₂O, 8.4 mg/l Titriplex III, 1.3 ml/l Synperonic 10% anti foam agent. The batch medium was supplemented with 5.4 mg/l Thiamin-HCl and 1.2 g/l L-leucine and L-proline respectively. The feed 1 solution contained 700 g/l glucose supplemented with 19.7 g/l MgSO₄*7H₂O. The alkaline solution for pH regulation was an aqueous 12.5% (w/v) NH₃ solution supplemented with 50 g/l L-leucine and 50 g/l L-proline respectively. All components were dissolved in deionized water.

The fermentation was carried out in a 10 l Biostat C DCU3 fermenter (Sartorius, Melsungen, Germany). Starting with 6.4 l sterile fermentation batch medium plus 300 ml inoculum from the pre-fermentation the batch fermentation was performed at 37° C., pH 6.9±0.2, 500 mbar and an aeration rate of 10 l/min. After the initially supplemented glucose was depleted the temperature was shifted to 28° C. and the fermentation entered the fed-batch mode. Here the relative value of dissolved oxygen (pO2) was kept at 50% (DO-stat, see e.g. Shay, L. K., et al., J. Indus. Microbiol. Biotechnol. 2 (1987) 79-85) by adding feed 1 in combination with constantly increasing stirrer speed (550 rpm to 1000 rpm within 10 hours and from 1000 rpm to 1400 rpm within 16 hours) and aeration rate (from 10 l/min to 16 l/min in 10 hours and from 16 l/min to 20 l/min in 5 hours). The supply with additional amino acids resulted from the addition of the alkaline solution, when the pH reached the lower regulation limit (6.70) after approximately 8 hours of cultivation. The expression of recombinant therapeutic protein was induced by the addition of 1 mM IPTG at an optical density of 70.

At the end of fermentation the cytoplasmatic and soluble expressed tetranectin-apolipoprotein A-I is transferred to insoluble protein aggregates, the so called inclusion bodies, with a heat step where the whole culture broth in the fermenter is heated to 50° C. for 1 or 2 hours before harvest (see e.g. EP-B 1 486 571). Thereafter, the content of the fermenter was centrifuged with a flow-through centrifuge (13,000 rpm, 13 l/h) and the harvested biomass was stored at −20° C. until further processing. The synthesized tetranectin-apolipoprotein A-I precursor proteins were found exclusively in the insoluble cell debris fraction in the form of insoluble protein aggregates, so-called inclusion bodies (IBs).

The synthesized fusion protein was found exclusively in the insoluble cell debris fraction in the form of insoluble protein aggregates, so-called inclusion bodies (IBs).

Samples drawn from the fermenter, one prior to induction and the others at dedicated time points after induction of protein expression are analyzed with SDS-Polyacrylamide gel electrophoresis. From every sample the same amount of cells (OD_{Target}=5) are resuspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 400 μL of SDS sample buffer (Laemmli, U.K., Nature 227 (1970) 680-685) are added. Samples are heated for 15 minutes at 95° C. under shaking to solubilize and reduce all proteins in the samples. After cooling to room temperature 5 μL of each sample are transferred to a 4-20% TGX Criterion Stain Free polyacrylamide gel (Bio-Rad). Additionally 5 μl molecular weight standard (Precision Plus Protein Standard, Bio-Rad) and 3 amounts (0.3 μl, 0.6 μl and 0.9 μl) quantification standard with known product protein concentration (0.1 μg/μl) are positioned on the gel.

The electrophoresis was run for 60 Minutes at 200 V and thereafter the gel was transferred the GelDOC EZ Imager (Bio-Rad) and processed for 5 minutes with UV radiation. Gel images were analyzed using Image Lab analysis software (Bio-Rad). With the three standards a linear regression curve was calculated with a coefficient of >0.99 and thereof the concentrations of target protein in the original sample was calculated.

### Fermentation Protocol 2:

For pre-fermentation a M9 medium according to Sambrook et al. (Molecular Cloning: A laboratory manual. Cold Spring Harbor Laboratory Press; 2nd edition (December 1989)) supplemented with about 1 g/l L-leucine, about 1 g/l L-proline and about 1 mg/l thiamine-HCl has been used.

For pre-fermentation 300 ml of modified M9-medium in a 1000 ml Erlenmeyer-flask with baffles was inoculated from agar plate or with 1-2 ml out of a primary seed bank ampoule. The cultivation was performed on a rotary shaker for 13 hours at 37° C. until an optical density (578 nm) of 1-3 was obtained.

For fermentation and high yield expression of tetranectin-apolipoprotein A-I the following batch medium and feeds were used:

8.85 g/l glucose, 63.5 g/l yeast extract, 2.2 g/l NH₄Cl, 1.94 g/l L-leucine, 2.91 g/l L-proline, 0.74 g/l L-methionine, 17.3 g/l KH₂PO₄*H2_{O}, 2.02 g/l MgSO₄*7H₂O, 25.8 mg/l Thiamin-HCl, 1.0 ml/l Synperonic 10% anti foam agent. The feed 1 solution contained 333 g/l yeast extract and 333 g/l 85%-glycerol supplemented with 1.67 g/l L-methionine and 5 g/l L-leucine and L-proline each. The feed 2 was a solution of 600 g/l L-Proline. The alkaline solution for pH regulation was a 10% (w/v) KOH solution and as acid a 75% glucose solution was used. All components were dissolved in deionized water.

The fermentation was carried out in a 10 l Biostat C DCU3 fermenter (Sartorius, Melsungen, Germany). Starting with 5.15 l sterile fermentation batch medium plus 300 ml inoculum from the pre-fermentation the fed-batch fermentation was performed at 25° C., pH 6.7±0.2, 300 mbar and an aeration rate of 10 l/min. Before the initially supplemented glucose was depleted the culture reached an optical density of 15 (578 nm) and the fermentation entered the fed-batch mode when feed 1 was started with 70 g/h. Monitoring the glucose concentration in the culture the feed 1 was increased to a maximum of 150 g/h while avoiding glucose accumulation and keeping the pH near the upper regulation limit of 6.9. At an optical density of 50 (578 nm) feed 2 was started with a constant feed rate of 10 ml/h. The relative value of dissolved oxygen (pO₂) was kept above 50% by increasing stirrer speed (500 rpm to 1500 rpm), aeration rate (from 10 l/min to 20 l/min) and pressure (from 300 mbar to 500 mbar) in parallel. The expression of recombinant therapeutic protein was induced by the addition of 1 mM IPTG at an optical density of 90.

Seven samples drawn from the fermenter, one prior to induction and the others at dedicated time points after induction of protein expression are analyzed with SDS-Polyacrylamide gel electrophoresis. From every sample the same amount of cells (OD_{Target}=5) are resuspended in 5 mL PBS buffer and disrupted via sonication on ice. Then 100 μL of each suspension are centrifuged (15,000 rpm, 5 minutes) and each supernatant is withdrawn and transferred to a separate vial. This is to discriminate between soluble and insoluble expressed target protein. To each supernatant (=soluble) fraction 300 μL and to each pellet (=insoluble) fraction 200 μL of SDS sample buffer (Laemmli, U.K., Nature 227 (1970) 680-685) are added. Samples are heated for 15 minutes at 95° C. under shaking to solubilize and reduce all proteins in the samples. After cooling to room temperature 5 μL of each sample are transferred to a 10% Bis-Tris polyacrylamide gel (Novagen). Additionally 5 μl molecular weight standard (Precision Plus Protein Standard, Bio-Rad) and 3 amounts (0.3 μl, 0.6 μl and 0.9 μl) quantification standard with known product protein concentration (0.1 μg/μl) are positioned on the gel.

The electrophoresis was run for 35 minutes at 200 V and then the gel was stained with Coomassie Brilliant Blue R dye, destained with heated water and transferred to an optical densitometer for digitalization (GS710, Bio-Rad). Gel images were analyzed using Quantity One 1-D analysis software (Bio-Rad). With the three standards a linear regression curve is calculated with a coefficient of >0.98 and thereof the concentrations of target protein in the original sample was calculated.

At the end of fermentation the cytoplasmatic and soluble expressed tetranectin-apolipoprotein A-I is transferred to insoluble protein aggregates, the so called inclusion bodies (IBs), with a heat step where the whole culture broth in the fermenter is heated to 50° C. for 1 or 2 hours before harvest (see e.g. EP-B 1 486 571). After the heat step the synthesized tetranectin-apolipoprotein A-I precursor proteins were found exclusively in the insoluble cell debris fraction in the form of IBs.

The contents of the fermenter are cooled to 4-8° C., centrifuged with a flow-through centrifuge (13,000 rpm, 13 l/h) and the harvested biomass is stored at −20° C. until further processing. The total harvested biomass yield ranged between 39 g/l and 90 g/l dry matter depending on the expressed construct.

### Example 3

### Preparation of Tetranectin-Apolipoprotein A-I

Inclusion body preparation was carried out by resuspension of harvested bacteria cells in a potassium phosphate buffered solution or a Tris buffered solution (0.1 M, supplemented with 1 mM MgSO₄, pH 6.5). After the addition of DNAse the cell were disrupted by homogenization at a pressure of 900 bar. A buffer solution comprising 1.5 M NaCl and 60 mM EDTA was added to the homogenized cell suspension. After the adjustment of the pH value to 5.0 with 25% (w/v) HCl the final inclusion body slurry was obtained after a further centrifugation step. The slurry was stored at −20° C. in single use, sterile plastic bags until further processing.

The inclusion body slurry (about 15 kg) was solubilized in a guanidinium hydrochloride solution (150 l, 6.7 M). After clarification of the solubilisate by depth filtration, the solution was applied to a Zn-chelate affinity chromatography material. The fusion polypeptide was purified by Zn-chelate chromatography material and cleaved by IgA protease. Thereafter the polypeptide was further purified with an anion exchange chromatography and a cation exchange chromatography step. These steps were performed in a urea containing solution (7 M), i.e. under denaturing conditions. These steps were used for the removal of polypeptide fragments, endotoxins, and further impurities. A diafiltration into 6.7 M guanidinium hydrochloride containing solution was carried out. The obtained final solution contains denatured tetranectin-apolipoprotein A-I.

### Example 4

### Refolding and Lipidation of Tetranectin-Apolipoprotein A-I

### a) General Method

Pure crystalline POPC or DPPC (Lipoid, Switzerland) have been dissolved in an aqueous buffer (lipidation buffer) containing cholate in a molar ratio phospholipid:cholate of 1:1.35. The mixtures have been incubated under nitrogen atmosphere and protected from light at room temperature (POPC) or at 55° C. (DPPC) until a clear solution has been obtained. The clear lipid-cholate solution is cooled to 4° C. (POPC) or stored at 41° C. (DPPC). Purified tetranectin-apolipoprotein A-I has been added at 4° C. (POPC) or 41° C. (DPPC) at a defined apolipoprotein:phospholipid ratio. For lipid particle formation the reaction mixture was incubated over night at 4° C. (POPC) or 41° C. (DPPC) under nitrogen atmosphere and protected from light. Finally, cholate was removed by extensive dialysis (4° C./41° C.) against lipidation buffer. Finally samples were centrifuged to remove precipitated material.

Cholate solubilized lipid solutions containing pure POPC or pure DPPC have been prepared as described above. Lipid mixtures were prepared by combining the lipid solutions at the desired ratio followed by storage at the respective Tₘ (Tₘ=phase transition temperature). Lipid particle formation of tetranectin-apolipoprotein A-I was performed as described for pure lipid solutions but at the respective Tₘ of the lipid mixture chosen.

The following lipidation buffers have been tested:
1. 50 mM potassium phosphate buffer supplemented with 250 mM arginine hydrochloride, 7.5% sucrose at pH 7.5
2. 50 mM dipotassium hydrogen phosphate buffer supplemented with 250 mM arginine hydrochloride, 7.5% sucrose, 10 mM methionine at pH 7.5
3. 250 mM tris-hydroxylamino methane (TRIS) supplemented with 140 mM NaCl, 10 mM methionine at pH 7.5
4. 50 mM dipotassium hydrogen phosphate buffer supplemented with 250 mM arginine hydrochloride, 7% trehalose, 10 mM methionine at pH 7.5.

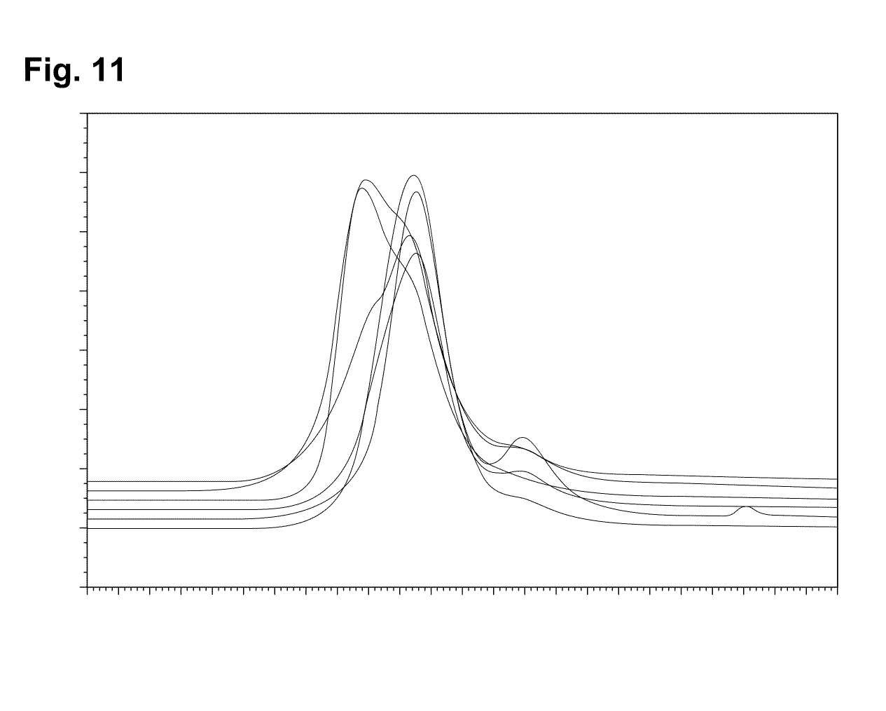

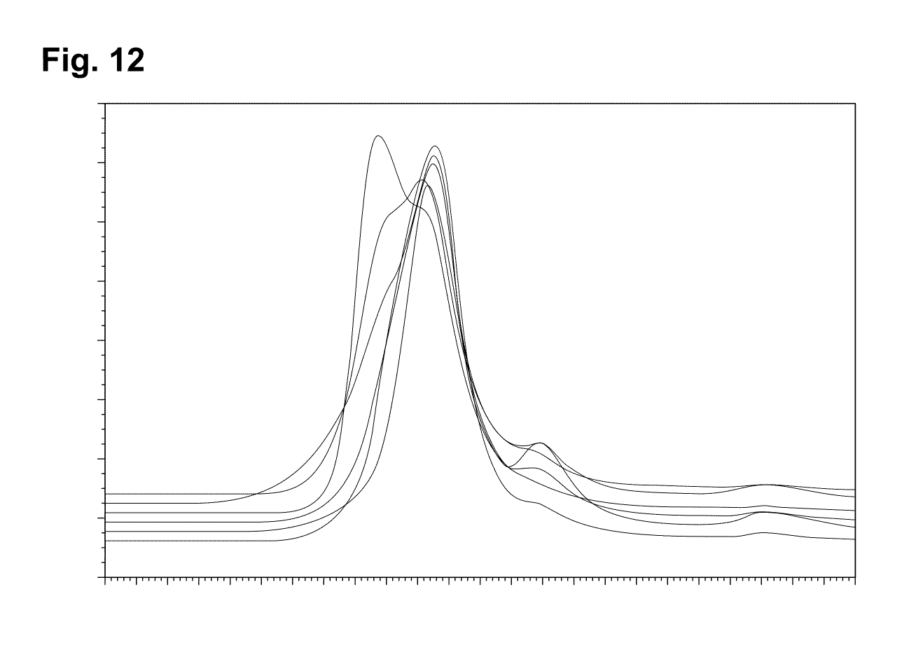

The homogeneity of the lipid particles formed from tetranectin-apolipoprotein A-I samples has been assessed by analytical SEC (FIGS. 11 and 12). Overall, the choice of the lipidation buffer has only a minor effect compared to the choice of phospholipid. DPPC-lipid particles elute as one main peak, whereas POPC-lipid particles shows a two peak pattern. The choice of lipidation buffer was influenced by the purification process of the apolipoprotein and the supply of stabilized lipid-free apolipoprotein. Lipid particle formation was shown to be feasible irrespective of the lipidation buffer. Among various buffers tested the most appropriate lipidation buffer was identified to be 250 mM Tris, 140 mM NaCl, 10 mM methionine, pH 7.5.

Lipidation mixtures contained a defined amount of apolipoprotein each and the amount of phospholipid, e.g. POPC, was calculated accordingly. All calculations of the molar amount of lipid were based on the tetranectin-apolipoprotein A-I monomer.

### b) POPC and Cholate

TABLE 6
Lipid particle formation with tetranectin-
apolipoprotein A-I as example using pure
POPC. Molar ratios apolipoprotein:phospholipid
are calculated for the protein monomer. Controls:
apolipoprotein incubated without addition
of lipid (pure Apo) and lipid without apolipoprotein
(no Apo).

| molar ratio | observa- | | | |
|---|---|---|---|---|
| apolipo- | tion after | protein conc. | protein conc. | |
| protein:phos- | overnight | before dialysis | after dialysis | observation |
| pholipid | incubation | [mg/ml] | [mg/ml] | after dialysis |
| 1:320 | clear | 0.67 | n.d. | turbid |
| 1:160 | clear | 1.34 | 1.47 | clear |
| 1:80 | clear | 2.68 | 2.6 | clear |
| 1:40 | clear | 5.36 | 4.87 | clear |
| 1:20 | turbid | 10.73 | 5.02 | turbid* |
| only Apo | turbid | 2.68 | 0.51 | turbid* |
| no Apo | clear | — | — | clear |

*clear after centrifugation

The molar ratios from 1:40 to 1:160 remain clear during the whole process. Neither turbidity through excess phospholipid nor protein precipitation was observed.

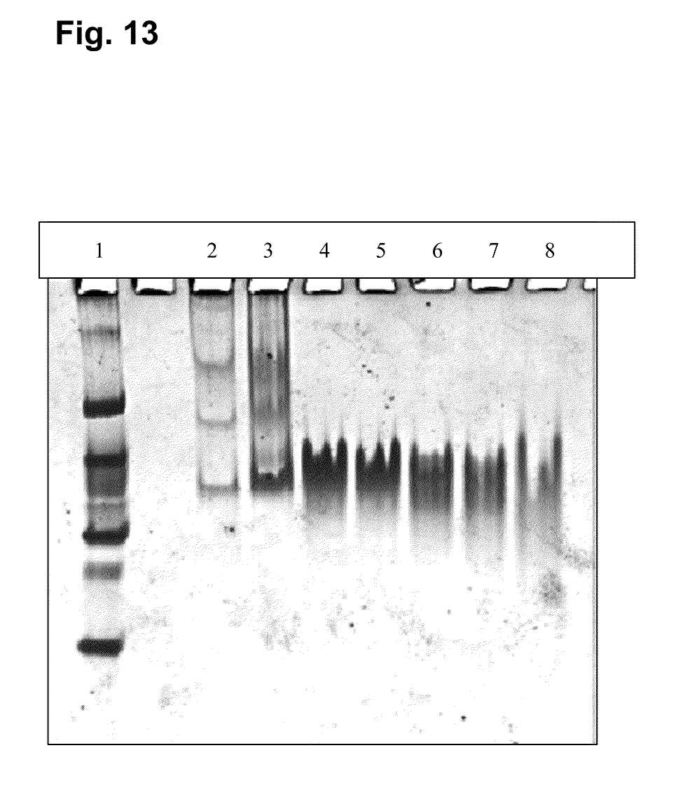

Lipid particle samples have been analyzed by native PAGE (see FIG. 13). The most homogeneous band pattern was found with the sample 1:80 (lane 4). In addition 1× freeze/thaw (−80° C.) did not alter appearance of the sample (lane 5). The band patterns of samples 1:320 and 1:160 indicate an inhomogeneous product resulting in multiple bands (lane 2 and 3). Samples 1:40 and also 1:20 have additional bands below the main product band (lane 6 and 7). The migration pattern of pure tetranectin-apolipoprotein A-I is shown in lane 8 of FIG. 13.

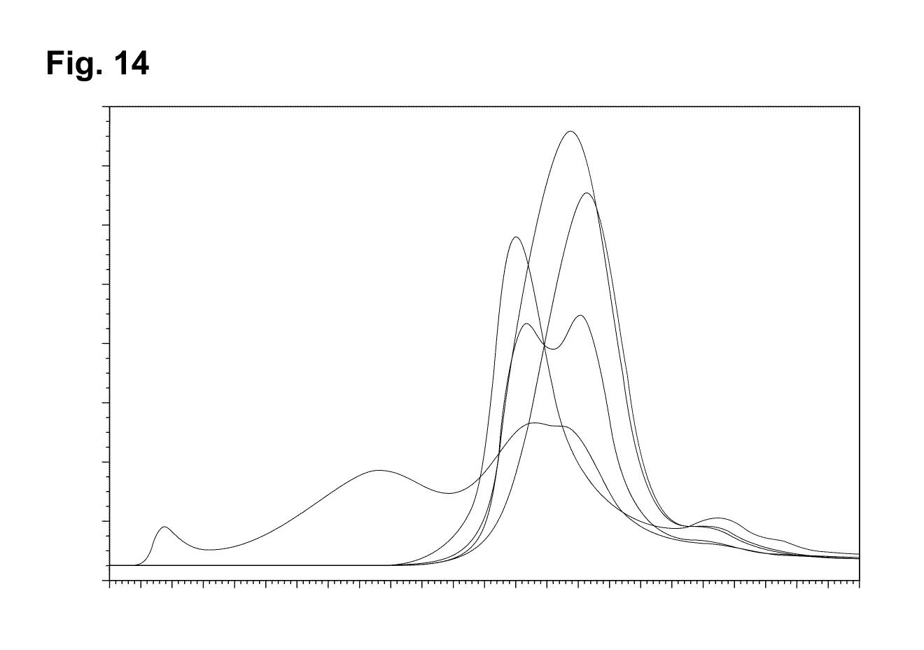

SEC-MALLS analysis was used to gain more detailed information on the homogeneity of the lipid particles and their apolipoprotein-phospholipid composition (protein-conjugate analysis). FIG. 14 shows the chromatogram of SEC resolved samples (UV280 detection). Here the 1:160 sample is divided into three separated peaks. The 1:80 sample appeared to contain at least two species of different size as displayed as double peak. The peak obtained from sample 1:20 shows the most homogeneous product.

The experiment was carried out using tetranectin-apolipoprotein A-I (3.84 mg/ml; 10 mg per sample) and the molar ratio apolipoprotein:phospholipid was increased from 1:40 to 1:80 in steps of 5. At molar ratios below 1:40 the lipid particle formation is incomplete. Molar ratios above 1:80 are excluded experimentally: after removal of cholate by dialysis the samples became turbid. Moreover the lipid particles became more inhomogeneous at higher lipid ratios.

TABLE 7
Lipid particle formation of tetranectin-apolipoprotein A-I using
pure POPC. Molar ratio apolipoprotein:phospholipid has been calculated
based on the tetranectin-apolipoprotein A-I monomer.

| molar ratio | | | | |
|---|---|---|---|---|
| apolipo- | protein conc. | protein conc. | | |
| protein:phos- | before dialysis | after dialysis | yield | observation |
| pholipid | [mg/ml]* | [mg/ml]* | [%] | after dialysis |
| 1:40 | 3.5 | 2.67 | 76 | precipitation |
| 1:45 | 3.5 | 2.74 | 78 | precipitation |
| 1:50 | 3.5 | 2.94 | 84 | precipitation |
| 1:55 | 3.5 | 3.05 | 87 | precipitation |
| 1:60 | 3.5 | 3.19 | 91 | precipitation |
| 1:65 | 3.5 | 3.34 | 95 | precipitation |
| 1:70 | 3.5 | 3.52 | 100** | |
| 1:75 | 3.5 | 3.56 | 100** | |
| 1:80 | 3.5 | 3.57 | 100** | |

*volume before and after dialysis 2.6 ml
**within SD of the method

During incubation at the transition temperature of −3° C. all samples remained optically clear. After removal of cholate by dialysis increasing turbidity of the samples 1:40 to 1:65 was observed. Precipitate could be removed by centrifugation and the samples remained clear afterwards.

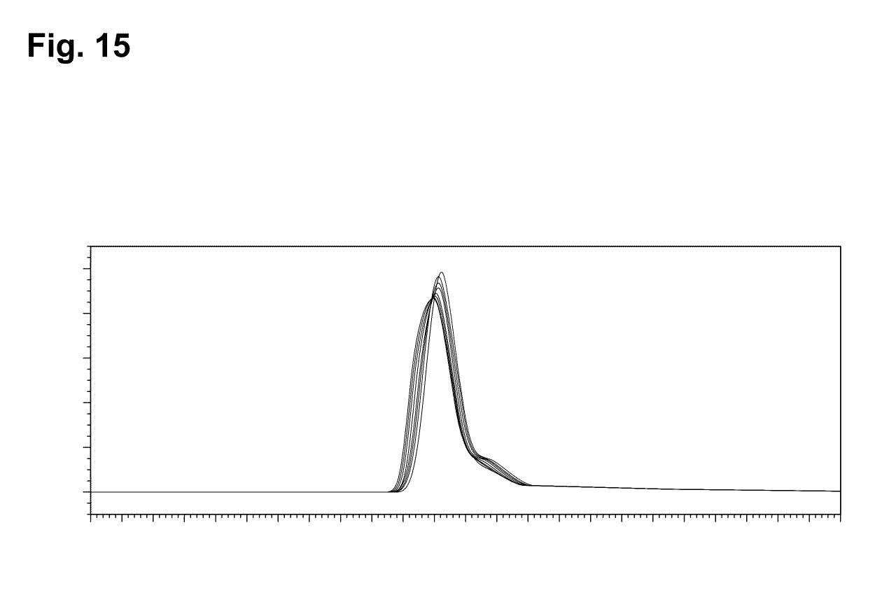

SEC-MALLS analysis was used to gain detailed information on the homogeneity of the formed lipid particles and their apolipoprotein-phospholipid composition (protein-conjugate analysis). All lipid particles were comparably homogeneous on analytical size exclusion chromatography (SEC; FIG. 15) displaying a minor post peak which is more pronounced at lower molar ratios. In addition, there is a noticeable shift in the peak pattern at higher molar ratios towards higher molecular weights. The respective retention times are given in Table 8.

TABLE 8
Summary of size exclusion chromatography results; percentages were
calculated by integration of the area under the curve (AUC).

| | retention time | main | post | |
|---|---|---|---|---|
| | main peak | peak | peak | total area |
| UV280 | [min.] | [%] | [%] | [mAU*min] |
| POPC 1:40 | 56.2 | 89.3 | 10.7 | 322.3 |
| POPC 1:45 | 55.9 | 89.7 | 10.4 | 331.3 |
| POPC 1:50 | 55.8 | 90.0 | 10.0 | 333.2 |
| POPC 1:55 | 55.7 | 91.0 | 9.1 | 342.5 |
| POPC 1:60 | 55.6 | 90.8 | 9.2 | 331.7 |
| POPC 1:65 | 55.3 | 90.9 | 9.2 | 337.2 |
| POPC 1:70 | 55.2 | 91.1 | 8.9 | 326.5 |
| POPC 1:75 | 55.1 | 91.3 | 8.7 | 347.1 |
| POPC 1:80 | 54.8 | 92.0 | 8.0 | 347.8 |

The protein-conjugate analysis (summarized in Table 8) enables the calculation of the total molecular weight of the protein (MW protein) and the lipid component (MW lipid) for each lipid particle eluted from the SEC column. Based on the molecular weights of tetranectin-apolipoprotein A-I monomer (32.7 kDa) and POPC (760 Da) the composition of the lipid particle can be calculated (n protein and n POPC). The molecular weight of the apolipoprotein component found in the lipid particle main peak at all molar ratios was approximately 100 kDa corresponding to a tetranectin-apolipoprotein A-I trimer per lipid particle. The ratio n(POPC)/n(protein monomer) gives the number of POPC molecules per tetranectin-apolipoprotein A-I monomer in the lipid particle. The number of POPC molecules per tetranectin-apolipoprotein A-I monomer varies between 54 and 75 though molar ratios from 1:40 up to 1:80 have been applied. The value % protein is a parameter for the degree of lipidation. The lower the percentage of the protein in the lipid particle, the higher the degree of lipidation.

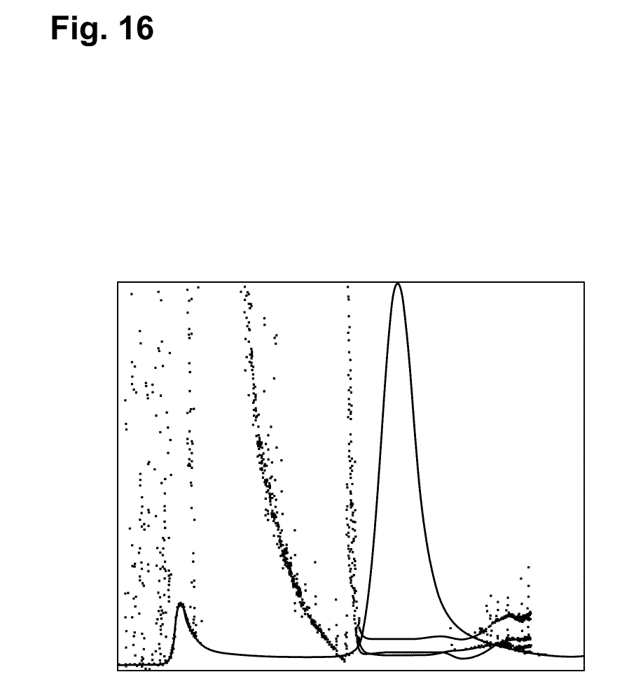

TABLE 9
Summary of protein conjugate analysis of lipid particles of POPC
and tetranectin-apolipoprotein A-I as shown in FIG. 16.

| | | MW total | MW Protein | | MW lipid | n | n (POPC)/ | |
|---|---|---|---|---|---|---|---|---|
| | | [kDa] | [kDa] | n (monomer) | [kDa] | (POPC) | n (monomer) | % protein |
| 1:40 | Main peak | 238 | 104 | 3.3 | 135 | 178 | 54 | 44 |
| | Post peak | 230 | 148 | 4.6 | 81 | 107 | 23 | 65 |
| 1:45 | Main peak | 238 | 101 | 3.2 | 138 | 182 | 57 | 42 |
| | Post peak | 184 | 118 | 3.7 | 66 | 87 | 24 | 64 |
| 1:50 | Main peak | 244 | 100 | 3.1 | 143 | 188 | 61 | 41 |
| | Post peak | 187 | 118 | 3.7 | 70 | 92 | 25 | 63 |
| 1:55 | Main peak | 247 | 99 | 3.1 | 148 | 195 | 63 | 40 |
| | Post peak | 182 | 107 | 3.3 | 75 | 99 | 30 | 59 |
| 1:60 | Main peak | 248 | 98 | 3.1 | 150 | 197 | 64 | 40 |
| | Post peak | 183 | 106 | 3.3 | 76 | 100 | 30 | 58 |
| 1:65 | Main peak | 255 | 97 | 3.0 | 158 | 208 | 69 | 38 |
| | Post peak | 191 | 103 | 3.2 | 88 | 116 | 36 | 54 |
| 1:70 | Main peak | 260 | 97 | 3.0 | 163 | 214 | 71 | 37 |
| | Post peak | 196 | 100 | 3.1 | 95 | 125 | 40 | 51 |
| 1:75 | Main peak | 266 | 99 | 3.1 | 168 | 221 | 71 | 37 |
| | Post peak | 208 | 118 | 3.7 | 91 | 120 | 32 | 56 |
| 1:80 | Main peak | 275 | 99 | 3.1 | 176 | 232 | 75 | 36 |
| | Post peak | 215 | 112 | 3.5 | 103 | 136 | 39 | 52 |

### c) DPPC and Cholate

Prior to lipidation the tetranectin-apolipoprotein A-I was dialyzed against 50 mM KH₂PO₄, 250 mM arginine hydrochloride, 7% trehalose, 10 mM methionine at pH 7.5. Tetranectin-apolipoprotein A-I (3.84 mg/ml, 3 mg per sample) has been lipidated using molar ratios from 1:60 to 1:100 increasing lipid concentrations in steps of 5. The lipidation buffer was 250 mM Tris-HCl, 140 mM NaCl, 10 mM methionine, pH 7.5.

TABLE 10
Sample overview of lipid particles of apolipoprotein with DPPC.

| molar ratio | | |
|---|---|---|
| apolipo- | observation | yield based on |
| protein:phos- | after o/n | protein |
| pholipid* | incubation | [%] |
| 1:20 | clear | 85 |
| 1:40 | clear | 88 |
| 1:60 | clear | 89 |
| 1:80 | clear | 91 |
| 1:100 | clear | 94 |
| only Apo | clear | 86 |
| no Apo | clear | DPPC precipitated |

*calculated for protein monomer

During lipid particle formation neither precipitation of protein nor turbidity through excess lipid was observed. The yield of tetranectin-apolipoprotein A-I in the final product was higher the more DPPC was used for lipidation.

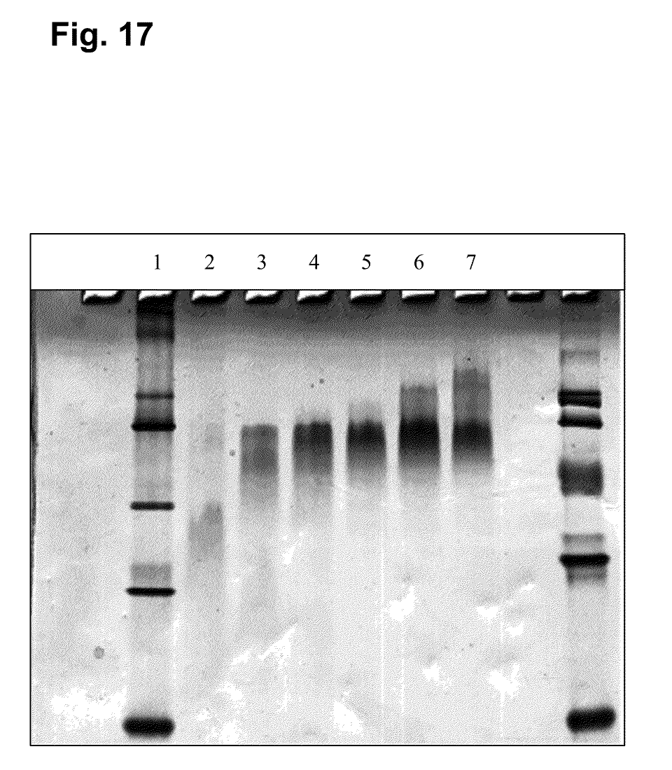

Residual lipid-free apolipoprotein was found in the 1:20 sample on native PAGE (lane 3, FIG. 17). The 1:40 and 1:60 sample look most homogeneous (lanes 4 and 5) on native PAGE whereas the 1:80 and 1:100 samples contain additional higher molecular bands above the main lipid particle band (lanes 6 and 7).

SEC-MALLS protein conjugate analysis was used to characterize the composition of the lipid particles obtained after DPPC lipid particle formation (MW DPPC: 734 Da). Homogeneous SEC peaks were obtained at molar ratios of 1:80 and below. At higher lipid ratios a pre-peak emerged (see e.g. 1:90 sample in Table 11).

TABLE 11
Summary SEC-MALLS protein conjugate analysis of lipid particles
of DPPC and tetranectin-apolipoprotein A-I.

| molar ratio | | | MW Protein | | | n (DPPC)/ | |
|---|---|---|---|---|---|---|---|
| apolipoprotein:phospholipid | peak | MW total [kDa] | [kDa] | n (protein) | MW lipid [kDa] | n (protein) | % protein |
| 1:60 | 1 | 724 | 298 | 9.0 | 425 | 193 | 41.2 |
| 1:65 | 1 | 281 | 109 | 3.3 | 171 | 77 | 38.9 |
| 1:70 | 1 | 273 | 103 | 3.1 | 169 | 76 | 37.9 |
| 1:75 | 1 | 286 | 103 | 3.1 | 183 | 83 | 36.0 |
| 1:80 | 1 | 295 | 100 | 3.0 | 194 | 88 | 34.1 |
| 1:85 | 1 | 307 | 99 | 3.0 | 207 | 94 | 32.6 |
| 1:90 | 1 | 361 | 117 | 3.5 | 244 | 110 | 32.6 |
| | 2 | 319 | 101 | 3.0 | 217 | 98 | 31.8 |
| 1:95 | 1 | 397 | 134 | 4.0 | 262 | 118 | 33.8 |
| | 2 | 327 | 100 | 3.0 | 226 | 102 | 30.8 |
| 1:100 | 1 | 405 | 132 | 4.0 | 273 | 123 | 32.6 |
| | 2 | 344 | 101 | 3.0 | 243 | 110 | 29.3 |

The highest degree of lipidation (lowest percentage of protein) is found with the 1:80 to 1:90 molar ratios. In addition DLS revealed most homogeneous particle formation at ratios 1:80 to 1:90 (>98%) at a particle size of 14-17 nm.

### d) 75% DPPC/25% POPC

The lipid particle formation was carried out accordingly as reported in items a) to c) of this example with the following parameters:
Protein: tetranectin-apolipoprotein A-I at 3.84 mg/ml, 3 mg per sample
Lipidation buffer: 250 mM Tris-HCl, 140 mM NaCl, 10 mM methionine pH 7.5
Lipidation: at 34° C.
Dialysis: at 4° C.
Molar ratios tested: 1:60 to 1:100 with increasing the lipid in steps of 5

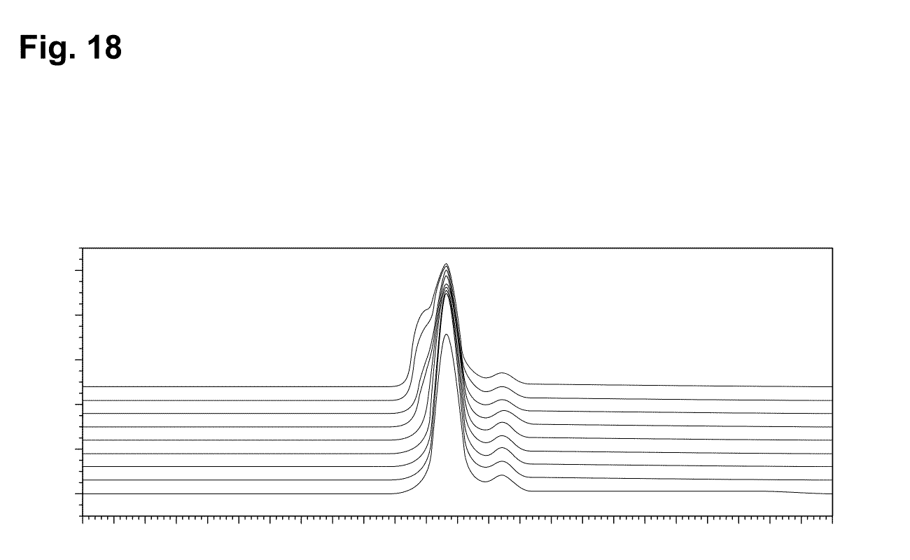

Lipid particle formation was straight forward and comparable to the process using pure lipids. All samples remained clear during the process and dialysis. The yield of lipid particles was similar for all ratios tested (˜85%). SEC-MALLS analysis showed that the molar ratio of 1:80 resulted in the most homogeneous lipid particles with 90.9% main peak, no pre-peak and 9.1% post-peak. Protein conjugate analysis revealed the presence of one tetranectin-apolipoprotein A-I trimer per lipid particle in the main species of all samples (see FIG. 18 and Tables 12 and 13).

TABLE 12
Summary of SEC results; percentages were
calculated by integration of the AUC.

| | Retention | Pre | Main | Post | total |
|---|---|---|---|---|---|
| | time Main | peak | peak | peak | [mAU * |
| UV280 | peak | [%] | [%] | [%] | min] |
| 75/25 DPPC/POPC 1:60 | 58.3 | — | 89.7 | 10.3 | 360.5 |
| 75/25 DPPC/POPC 1:65 | 58.3 | — | 89.2 | 10.8 | 383.7 |
| 75/25 DPPC/POPC 1:70 | 58.3 | — | 89.5 | 10.5 | 376.8 |
| 75/25 DPPC/POPC 1:75 | 58.4 | — | 90.3 | 9.7 | 367.0 |
| 75/25 DPPC/POPC 1:80 | 58.3 | — | 90.9 | 9.1 | 383.5 |
| 75/25 DPPC/POPC 1:85 | 58.2 | 10.4 | 79.5 | 10.1 | 356.4 |
| 75/25 DPPC/POPC 1:90 | 58.3 | 10.2 | 81.5 | 8.3 | 344.6 |
| 75/25 DPPC/POPC 1:95 | 58.0 | 16.9 | 74.9 | 8.2 | 377.4 |
| 75/25 DPPC/POPC 1:100 | 58.0 | 21.0 | 70.4 | 7.7 | 365.0 |

TABLE 13
Summary protein-conjugate analysis of 75% DPPC/25% POPC and
tetranectin-apolipoprotein A-I lipid particles.

| | | | MW protein | n (protein | MW lipid | | n (lipid)/ | |
|---|---|---|---|---|---|---|---|---|
| | | MW total | [kDa] | monomer) | [kDa] | n (lipid) | n (monomer) | % protein |
| 1:60 | Main peak | 257 | 96 | 3.0 | 161 | 217 | 72 | 37 |
| | Post peak | 92 | 75 | 2.3 | 17 | 23 | 10 | 82 |
| 1:65 | Main peak | 263 | 95 | 3.0 | 167 | 226 | 76 | 36 |
| | Post peak | 116 | 102 | 3.2 | 14 | 19 | 6 | 88 |
| 1:70 | Main peak | 268 | 95 | 3.0 | 173 | 234 | 79 | 35 |
| | Post peak | 93 | 83 | 2.6 | 10 | 14 | 5 | 89 |
| 1:75 | Main peak | 275 | 95 | 3.0 | 180 | 243 | 82 | 34 |
| | Post peak | 98 | 82 | 2.6 | 16 | 22 | 8 | 84 |
| 1:80 | Main peak | 279 | 95 | 3.0 | 184 | 248 | 84 | 34 |
| | Post peak | 97 | 86 | 2.7 | 11 | 15 | 6 | 89 |
| 1:85 | Pre peak | 329 | 104 | 3.3 | 224 | 302 | 93 | 32 |
| | Main peak | 291 | 96 | 3.0 | 195 | 263 | 88 | 33 |
| | Post peak | 129 | 107 | 3.3 | 22 | 30 | 9 | 83 |
| 1:90 | Pre peak | 443 | 107 | 3.3 | 237 | 320 | 96 | 31 |
| | Main peak | 293 | 95 | 3.0 | 197 | 266 | 90 | 33 |
| | Post peak | 126 | 102 | 3.2 | 25 | 34 | 11 | 81 |
| 1:95 | Pre peak | 384 | 110 | 3.4 | 274 | 370 | 108 | 29 |
| | Main peak | 303 | 96 | 3.0 | 207 | 280 | 93 | 32 |
| | Post peak | 130 | 103 | 3.2 | 27 | 36 | 11 | 79 |
| 1:100 | Pre peak | 398 | 111 | 3.5 | 287 | 388 | 112 | 28 |
| | Main peak | 310 | 96 | 3.0 | 213 | 288 | 96 | 31 |
| | Post peak | 122 | 86 | 2.7 | 36 | 49 | 18 | 71 |

### e) 50% DPPC/50% POPC

The lipid particle formation was carried out accordingly as reported in items a) to c) of this example with the following parameters:
Protein: tetranectin-lipoprotein A-I at 3.84 mg/ml, 3 mg per sample
Lipidation buffer: 250 mM Tris-HCl, 140 mM NaCl, 10 mM methionine, pH 7.5
Lipidation: at 27° C.
Dialysis: at room temperature
Molar ratios tested: 1:60 to 1:100 with increasing lipid in steps of 5

All samples remained clear during the process and dialysis. The yield of lipid particles was similar for all ratios tested.

TABLE 14
Summary of SEC results; percentages were
calculated by integration of the AUC.

| | Retention time | Pre | Main | Post | |
|---|---|---|---|---|---|
| | Main peak | peak | peak | peak | total |
| UV280 | [min] | [%] | [%] | [%] | [mAU*min] |
| 50/50 DPPC/ | 58.2 | — | 88.9 | 11.1 | 341.3 |
| POPC 1:60 | | | | | |
| 50/50 DPPC/ | 58.3 | — | 89.3 | 10.7 | 349.6 |
| POPC 1:65 | | | | | |
| 50/50 DPPC/ | 58.3 | — | 89.9 | 10.1 | 336.9 |
| POPC 1:70 | | | | | |
| 50/50 DPPC/ | 58.2 | 6.1 | 84.3 | 9.6 | 347.4 |
| POPC 1:75 | | | | | |
| 50/50 DPPC/ | 58.1 | 8.5 | 82.2 | 9.3 | 356.9 |
| POPC 1:80 | | | | | |
| 50/50 DPPC/ | 58.0 | 11.3 | 79.8 | 8.9 | 352.7 |
| POPC 1:85 | | | | | |
| 50/50 DPPC/ | 58.0 | 14.4 | 77.1 | 8.5 | 356.5 |
| POPC 1:90 | | | | | |
| 50/50 DPPC/ | 58.0 | 19.3 | 72.6 | 8.1 | 367.0 |
| POPC 1:95 | | | | | |
| 50/50 DPPC/ | 57.9 | 36.6 | 65.8 | 7.6 | 365.3 |
| POPC 1:100 | | | | | |

Using a lipid mixture of 50% DPPC and 50% POPC for lipid particle formation of tetranectin-apolipoprotein A-I the most homogeneous product was obtained at a molar ratio of 1:70 (see Table 14). The product was 89.9% pure with respect to the main peak and contained one single tetranectin-apolipoprotein A-I trimer (see Table 15).

TABLE 15
Summary protein conjugate analysis of lipid particles with 50%
DPPC/50% POPC and tetranectin-apolipoprotein A-I.

| | | | | n (protein | | | n (lipid)/ | |
|---|---|---|---|---|---|---|---|---|
| | | MW total | MW protein | monomer) | MW lipid | n (lipid) | n (monomer) | % protein |
| 1:60 | Main peak | 331 | 124 | 3.9 | 207 | 277 | 71 | 38 |
| | Post peak | 131 | 106 | 3.3 | 24 | 32 | 10 | 81 |
| 1:65 | Main peak | 264 | 95 | 2.9 | 169 | 226 | 78 | 36 |
| | Post peak | 127 | 112 | 3.5 | 16 | 21 | 6 | 88 |
| 1:70 | Main peak | 273 | 96 | 3.0 | 178 | 238 | 79 | 35 |
| | Post peak | 258 | 213 | 6.7 | 45 | 60 | 9 | 82 |
| 1:75 | Pre peak | 319 | 108 | 3.4 | 211 | 282 | 83 | 34 |
| | Main peak | 271 | 93 | 2.9 | 178 | 238 | 82 | 34 |
| | Post peak | 126 | 106 | 3.3 | 20 | 27 | 8 | 84 |
| 1:80 | Pre peak | 333 | 108 | 3.4 | 225 | 301 | 89 | 32 |
| | Main peak | 278 | 95 | 2.9 | 184 | 246 | 85 | 34 |
| | Post peak | 122 | 100 | 3.1 | 21 | 28 | 9 | 83 |
| 1:85 | Pre peak | 359 | 109 | 3.4 | 250 | 335 | 98 | 30 |
| | Main peak | 284 | 94 | 2.9 | 189 | 253 | 87 | 33 |
| | Post peak | 132 | 118 | 3.7 | 14 | 19 | 5 | 89 |
| 1:90 | Pre peak | 373 | 109 | 3.4 | 264 | 353 | 104 | 29 |
| | Main peak | 286 | 94 | 2.9 | 192 | 257 | 89 | 33 |
| | Post peak | 133 | 110 | 3.4 | 23 | 31 | 9 | 83 |
| 1:95 | Pre peak | 390 | 111 | 3.5 | 278 | 372 | 106 | 29 |
| | Main peak | 290 | 94 | 2.9 | 195 | 261 | 90 | 33 |
| | Post peak | 162 | 136 | 4.3 | 26 | 35 | 8 | 84 |
| 1:100 | Pre peak | 404 | 113 | 3.5 | 291 | 390 | 111 | 28 |
| | Main peak | 293 | 94 | 2.9 | 199 | 266 | 92 | 32 |
| | Post peak | 142 | 107 | 3.3 | 35 | 47 | 14 | 75 |

### f) 25% DPPC/75% POPC

The lipid particle formation was carried out accordingly as reported in items a) to c) of this example with the following parameters:
Protein: tetranectin-apolipoprotein A-I at 3.84 mg/ml, 3 mg per sample
Lipidation buffer: 250 mM Tris-HCl, 140 mM NaCl, 10 mM methionine, pH 7.5
Lipidation: at 18° C.
Dialysis: at room temperature
Molar ratios tested: 1:60 to 1:100 with increasing lipid in steps of 5

Lipid particle formation was straight forward and comparable to the process using pure lipids. All samples remained clear during the process and dialysis.

TABLE 16
Summary of SEC results; percentages were
calculated by integration of the AUC.

| | Retention time | Pre | Main | Post | |
|---|---|---|---|---|---|
| | Main peak | peak | peak | peak | total |
| UV280 | [min] | % | % | % | [mAU*min] |
| 25/75 DPPC/ | 58.2 | — | 90.2 | 9.8 | 342.6 |
| POPC 1:60 | | | | | |
| 25/75 DPPC/ | 58.2 | 4.6 | 85.9 | 9.4 | 345.6 |
| POPC 1:65 | | | | | |
| 25/75 DPPC/ | 58.1 | 8.8 | 82.3 | 8.9 | 353.2 |
| POPC 1:70 | | | | | |
| 25/75 DPPC/ | 58.0 | 9.0 | 82.4 | 8.6 | 357.5 |
| POPC 1:75 | | | | | |
| 25/75 DPPC/ | 57.9 | 10.8 | 81.2 | 8.0 | 356.7 |
| POPC 1:80 | | | | | |
| 25/75 DPPC/ | 57.9 | 21.2 | 71.0 | 7.8 | 366.3 |
| POPC 1:85 | | | | | |
| 25/75 DPPC/ | 57.8 | 26.1 | 66.4 | 7.5 | 357.8 |
| POPC 1:90 | | | | | |
| 25/75 DPPC/ | 57.7 | 32.7 | 60.5 | 6.8 | 365.9 |
| POPC 1:95 | | | | | |
| 25/75 DPPC/ | 57.6 | 36.1 | 57.5 | 6.4 | 373.4 |
| POPC 1:100 | | | | | |

Using a lipid mixture of 25% DPPC and 75% POPC for lipid particle formation of tetranectin-apolipoprotein A-I the most homogeneous product was obtained at a molar ratio of 1:60 (see Table 17). The product was 90.2% pure with respect to the main peak and contained one single tetranectin-apolipoprotein A-I trimer (see Table 15).

TABLE 17
Summary protein conjugate analysis of lipid particles of 25%
DPPC/75% POPC and tetranectin-apolipoprotein A-I.

| | | | | n (protein | | | n (lipid)/ | |
|---|---|---|---|---|---|---|---|---|
| | | MW total | MW protein | monomer) | MW lipid | n (lipid) | n (monomer) | % protein |
| 1:60 | Main peak | 254 | 100 | 3.1 | 153 | 203 | 66 | 40 |
| | Post peak | 127 | 110 | 3.4 | 17 | 23 | 7 | 86 |
| 1:65 | Pre peak | 272 | 132 | 4.1 | 141 | 187 | 46 | 48 |
| | Main peak | 259 | 100 | 3.1 | 159 | 211 | 68 | 39 |
| | Post peak | 183 | 131 | 4.1 | 7 | 9 | 2 | 95 |
| 1:70 | Pre peak | 280 | 121 | 3.8 | 159 | 211 | 56 | 43 |
| | Main peak | 264 | 99 | 3.1 | 165 | 219 | 71 | 38 |
| | Post peak | 119 | 105 | 3.3 | 14 | 19 | 6 | 88 |
| 1:75 | Pre peak | 291 | 109 | 3.4 | 183 | 243 | 71 | 37 |
| | Main peak | 268 | 98 | 3.1 | 170 | 226 | 73 | 37 |
| | Post peak | 120 | 101 | 3.2 | 19 | 25 | 8 | 84 |
| 1:80 | Pre peak | 311 | 114 | 3.6 | 197 | 261 | 73 | 37 |
| | Main peak | 276 | 96 | 3.0 | 176 | 234 | 78 | 36 |
| | Post peak | 137 | 127 | 4.0 | 10 | 13 | 3 | 93 |
| 1:85 | Pre peak | 331 | 115 | 3.6 | 216 | 287 | 80 | 35 |
| | Main peak | 278 | 98 | 3.1 | 180 | 239 | 77 | 35 |
| | Post peak | 139 | 117 | 3.7 | 22 | 29 | 8 | 85 |
| 1:90 | Pre peak | 345 | 113 | 3.5 | 232 | 308 | 88 | 33 |
| | Main peak | 285 | 98 | 3.1 | 187 | 248 | 80 | 34 |
| | Post peak | 143 | 110 | 3.4 | 33 | 44 | 13 | 77 |
| 1:95 | Pre peak | 363 | 115 | 3.6 | 248 | 329 | 91 | 32 |
| | Main peak | 292 | 97 | 3.0 | 194 | 257 | 86 | 33 |
| | Post peak | 155 | 122 | 3.8 | 33 | 44 | 12 | 79 |
| 1:100 | Pre peak | 377 | 117 | 3.7 | 260 | 345 | 93 | 31 |
| | Main peak | 298 | 98 | 3.1 | 200 | 265 | 86 | 33 |
| | Post peak | 160 | 114 | 3.6 | 46 | 61 | 17 | 71 |

### g) Lipid Particle Formation Using Zwittergent

The lipid particle formation was carried out accordingly as reported in items a) to c) of this example with the following parameters and the exception that cholate was replaced by the synthetic detergent Zwittergent:
Protein: tetranectin-apolipoprotein A-I at 23.5 mg/ml
Buffer: 50 mM Tris-HCl, 7.2 M guanidinium hydrochloride, 10 mM Methionine, pH 8
Lipidation buffer: 250 mM Tris-HCl, 140 mM NaCl, pH 7.5

100% POPC, molar ratio apolipoprotein:phospholipid=1:60

TABLE 18
Sample overview of various approaches and observations/
parameters of lipid particle formation.

| | | turbidity | | | | | | |
|---|---|---|---|---|---|---|---|---|
| | | dissolved | | after | volume after | c after dialysis | | yield |
| sample | detergent [%] | lipid | lipidation | dialysis | dialysis [ml] | [μg/ml] | [mg] TN-Apo A-I | [%] |

Zwittergent 3-8

| | | | | | | | | |
|---|---|---|---|---|---|---|---|---|
| 0.1 × | 0.8 | +++ | +++ | +++ | 2.1 | 2230.18 | 4.68 | 99.6 |
| CMC | | | | | | | | |
| 0.5 × | 4.2 | ++ | ++ | + | 2.9 | 1536.81 | 4.46 | 94.8 |
| CMC | | | | | | | | |
| 1 × CMC | 8.4 | + | + | + | 3 | 1475.07 | 4.43 | 94.2 |
| 2 × CMC | 16.7 | − | − | − | 4.3 | 1081.27 | 4.65 | 98.9 |
| 3 × CMC | 25.1 | − | − | − | 5.5 | 839.85 | 4.62 | 98.3 |

Zwittergent 3-10

| | | | | | | | | |
|---|---|---|---|---|---|---|---|---|
| 0.1 × | 0.1 | +++ | +++ | +++ | 2 | 2361.56 | 4.72 | 100.5 |
| CMC | | | | | | | | |
| 0.5 × | 0.6 | +++ | ++ | ++ | 2 | 2221.38 | 4.44 | 94.5 |
| CMC | | | | | | | | |
| 1 × CMC | 1.2 | ++ | + | + | 2.1 | 2267.16 | 4.76 | 101.3 |
| 2 × CMC | 2.5 | + | + | (+) | 2.3 | 2082.18 | 4.79 | 101.9 |
| 5 × CMC | 6.2 | − | − | − | 2.5 | 1941.61 | 4.85 | 103.3 |
| 10 × | 12.3 | − | − | − | 4 | 1073.92 | 4.30 | 91.4 |
| CMC | | | | | | | | |

Zwittergent 3-12

| | | | | | | | | |
|---|---|---|---|---|---|---|---|---|
| 0.1 × | 0.01 | +++ | +++ | +++ | 2 | 2722.85 | 5.45 | 115.9 |
| CMC | | | | | | | | |
| 1 × CMC | 0.1 | +++ | +++ | +++ | 2 | 2158.81 | 4.32 | 91.9 |
| 2 × CMC | 0.2 | +++ | +++ | ++ | 2 | 2636 | 5.27 | 112.2 |
| 20 × | 1.9 | + | + | + | 2.1 | 2525.69 | 5.30 | 112.8 |
| CMC | | | | | | | | |
| 100 × | 9.4 | − | − | − | 3.5 | 1567.85 | 5.49 | 116.8 |
| CMC | | | | | | | | |
| 300 × | 28.1 | − | − | − | 5.6 | 1069.04 | 5.99 | 127.4 |
| CMC | | | | | | | | |

Cholate

| | | | | | | | | |
|---|---|---|---|---|---|---|---|---|
| 0.1 × | 0.06 | +++ | +++ | +++ | 2 | 2323.09 | 4.65 | 98.9 |
| CMC | | | | | | | | |
| 0.5 × | 0.3 | + | − | − | 2 | 2301.15 | 4.60 | 97.9 |
| CMC | | | | | | | | |
| 1 × CMC | 0.6 | − | − | − | 2 | 2316.86 | 4.63 | 98.6 |
| 2 × CMC | 1.2 | − | − | − | 2.5 | 1178.72 | 2.95 | 62.7 |
| 5 × CMC | 3 | − | − | − | 2.5 | 2435.34 | 6.09 | 129.5 |
| 10 × | 6 | − | − | − | 3.5 | 1814.69 | 6.35 | 135.1 |
| CMC | | | | | | | | |

Lipid particles comprising tetranectin-apolipoprotein A-I have been analyzed on native PAGE. Lipid-free tetranectin-apolipoprotein A-I migrates at 140 kDa (lanes 1 in FIG. 19), whereas lipid particles show a characteristic shift to a higher molecular weight between 232 kDa and 440 kDa.

Lipid-free tetranectin-apolipoprotein A-I but no lipid particles were detected in all samples prepared with only 0.1×CMC of the respective detergent (FIG. 19, lanes 2, 8, 13, and 19). However, a detergent concentration of 0.5×CMC was sufficient for Zwittergent 3-8 and 3-10 to enable the lipid particle formation with tetranectin-apolipoprotein A-I (lanes 3, 9, and 14). With Zwittergent 3-12 lipid particle formation did not occur until a concentration of 2.0×CMC was reached (lane 21).

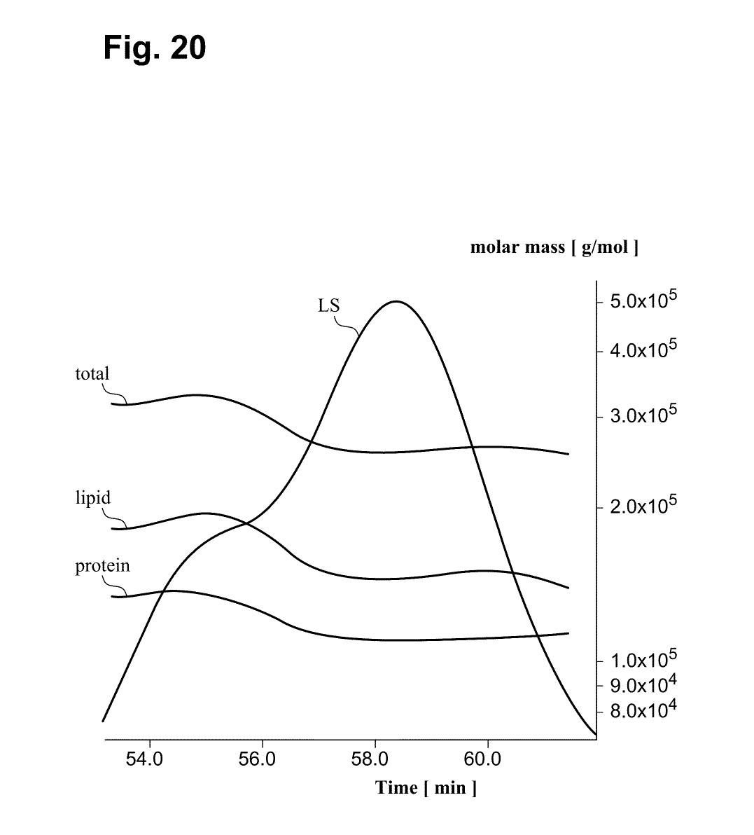

FIG. 20 shows the SEC-MALLS chromatogram of lipid particles comprising tetranectin-apolipoprotein A-I using 3×CMC Zwittergent 3-8 and POPC (molar ratio apolipoprotein:phospholipid=1:60). Results of the protein conjugate analysis are summarized in Table 18. The lipid particle fraction consists of two different species as displayed in two overlapping peaks in the SEC chromatogram. However, these two species are very similar, differentiating mainly in the number of tetranectin-apolipoprotein A-I molecules per particle (4.2 for peak 1 and 3.5 for peak 2).

TABLE 19
Summary of protein-conjugate analysis of lipid
particles formed in the presence of Zwittergent 3-8.

| | | | | | | | | | Rh (w) |
|---|---|---|---|---|---|---|---|---|---|
| | | | | n (protein | | | n (lipid)/ | | (QELS) |
| ×CMC | | MW total | MW protein | monomer) | MW lipid | n (lipid) | n (monomer) | % protein | [nm] |
| 2 | Pre peak | 345 | 147 | 4.6 | 198 | 261.5 | 57 | 42.5 | 7.7 |
| | Main peak | 268 | 113 | 3.6 | 154 | 203.2 | 56 | 42.4 | 6.5 |
| 3 | Pre peak | 323 | 134 | 4.2 | 188 | 249.9 | 60 | 41.6 | 7.4 |
| | Main peak | 257 | 110 | 3.5 | 146 | 192.9 | 55 | 43.0 | 6.5 |

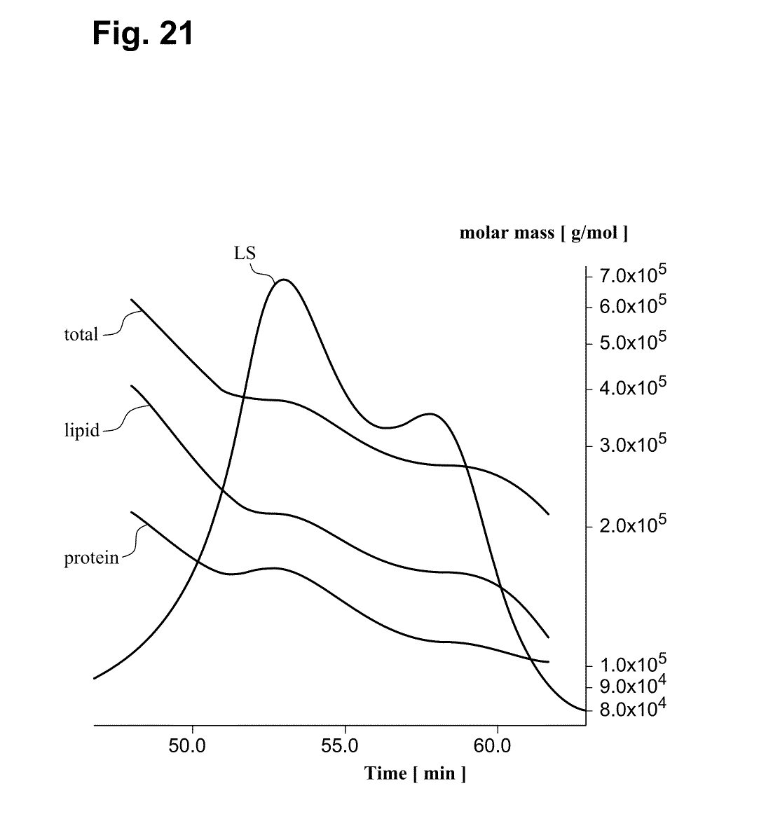

FIG. 21 shows the chromatogram of SEC-MALLS analysis and Table 19 the summary of the protein conjugate analysis for lipid particles comprising tetranectin-apolipoprotein A-I using 2×CMC Zwittergent 3-10 and POPC (molar ratio apolipoprotein:phospholipid=1:60). Both peaks contain lipid particles comprising 3.5 and 5 tetranectin-apolipoprotein A-I molecules, respectively.

TABLE 20
Summary of protein-conjugate analysis of lipid
particles formed in the presence of Zwittergent 3-10.

| | | | | | | | | | Rh (w) |
|---|---|---|---|---|---|---|---|---|---|
| | | | | n (protein | | | n (lipid)/ | | (QELS) |
| ×CMC | | MW total | MW protein | monomer) | MW lipid | n (lipid) | n (monomer) | % protein | [nm] |
| 2 | Pre peak | 373 | 161 | 5.0 | 211 | 278.7 | 56 | 43.2 | 7.8 |
| | Main peak | 272 | 112 | 3.5 | 159 | 210.3 | 60 | 41.4 | 6.6 |
| 5 | Pre peak | 345 | 150 | 4.7 | 195 | 256.6 | 55 | 43.6 | 7.5 |
| | Main peak | 263 | 112 | 3.5 | 151 | 199.1 | 57 | 42.6 | 6.6 |
| 10 | Pre peak | 405 | 151 | 4.7 | 253 | 334.1 | 71 | 37.4 | 7.9 |
| | Main peak | 265 | 110 | 3.3 | 154 | 203.2 | 58 | 41.8 | 6.5 |

The results of lipid particle formation comprising tetranectin-apolipoprotein A-I using Zwittergent 3-12 and POPC (molar ratio apolipoprotein:phospholipid=1:60) are summarized in Table 21. The lipid particle fraction consists of two different species as displayed in two overlapping peaks in the SEC chromatogram. However, these two species are very similar, differentiating mainly in the number of tetranectin-apolipoprotein A-I molecules per particle.

TABLE 21
Summary of protein-conjugate analysis of lipid
particles formed in the presence of Zwittergent 3-12.

| | | | | | | | | | Rh (w) |
|---|---|---|---|---|---|---|---|---|---|
| | | | | n (protein | | | n (lipid)/ | | (QELS) |
| ×CMC | | MW total | MW protein | monomer) | MW lipid | n (lipid) | n (monomer) | % protein | [nm] |
| 100 | Main peak | 487 | 342 | 10.7 | 145 | 191.3 | 18 | 70.2 | 11.9 |
| 300 | Main peak | 241 | 208 | 6.5 | 32 | 43.3 | 7 | 86.4 | 8.5 |

The results of lipid particle formation comprising tetranectin-apolipoprotein A-I using cholate and POPC (molar ratio apolipoprotein:phospholipid=1:60) are summarized in Table 21. The lipid particle fraction consists of two different species as displayed in two overlapping peaks in the SEC chromatogram. However, these two species are very similar, differentiating mainly in the number of tetranectin-apolipoprotein A-I molecules per particle.

TABLE 22
Summary of protein-conjugate analysis of lipid particles formed in
the presence of cholate.

| | | | | n (protein | | | n (lipid)/ | | Rh (w) |
|---|---|---|---|---|---|---|---|---|---|
| CMC | | MW total | MW protein | monomer) | MW lipid | n (lipid) | n (monomer) | % protein | (QELS) [nm] |
| 0.5 | Pre peak | 1295 | 461 | 14.5 | 829 | 1091 | 75 | 35.9 | 12.7 |
| | Main peak | 361 | 153 | 4.8 | 207 | 273 | 57 | 42.5 | 7.7 |
| | Post peak | 283 | 115 | 3.6 | 168 | 221 | 62 | 40.6 | 6.8 |
| 1 | Pre peak | 1050 | 414 | 12.9 | 623 | 836 | 65 | 39.5 | 11.8 |
| | Main peak | 337 | 154 | 4.8 | 182 | 240 | 50 | 45.9 | 7.6 |
| | Post peak | 284 | 121 | 3.8 | 162 | 214 | 56 | 42.7 | 6.9 |
| 2 | Pre peak | 332 | 143 | 4.5 | 188 | 248 | 55 | 43.2 | 7.3 |
| | Main peak | 269 | 111 | 3.5 | 158 | 209 | 60 | 41.2 | 6.5 |
| 5 | Pre peak | 314 | 143 | 4.5 | 171 | 225 | 50 | 45.6 | 7.5 |
| | Main peak | 278 | 118 | 3.7 | 158 | 208 | 56 | 42.7 | 6.8 |
| 10 | Pre peak | 292 | 135 | 4.2 | 156 | 206 | 50 | 46.3 | 7.3 |
| | Main peak | 271 | 115 | 3.6 | 155 | 204 | 57 | 42.6 | 6.6 |

### Example 5

### Rapid Dilution Method for Refolding and Lipid Particle Formation

### a) POPC and Sodium Cholate

Tetranectin-apolipoprotein A-I was expressed in E. coli and purified according to Examples 1 to 3 (protocol 1). After purification, the buffer was exchanged by diafiltration to a solution containing 250 mM Tris, 140 mM NaCl, 6.7 M guanidinium hydrochloride, pH 7.4. The protein concentration was adjusted to 28 mg/ml.

A lipid stock solution was prepared by dissolving 100 moles/l of POPC in a buffer containing 250 mM Tris-HCl, 140 mM NaCl, 135 mM sodium cholate, pH 7.4 at room temperature. The lipid stock solution was incubated for 2 hours at room temperature. Refolding buffer was prepared by diluting 77 ml of the lipid stock mixture into 1478 ml of 250 mM Tris-HCl, 140 mM NaCl, pH 7.4. This buffer was stirred for an additional 7 hours at room temperature.

Refolding and lipid particle formation was initiated by the addition of 162 ml tetranectin-apolipoprotein A-I in 250 mM Tris, 140 mM NaCl, 6.7 M guanidinium hydrochloride, pH 7.4 to refolding buffer. This results in a 1:10 dilution of the guanidinium hydrochloride. The solution was incubated at room temperature for 16 hours while constantly stirring. The removal of the detergent was carried out by diafiltration.

TABLE 23
Summary protein conjugate analysis of
lipid particle obtained by rapid dilution with POPC.

| | MW | MW | | MW | | | |
|---|---|---|---|---|---|---|---|
| | total | protein | n (protein | lipid | | n (lipid)/ | % |
| Peak | [kDa] | [kDa] | monomer) | [kDa] | n (lipid) | n (protein) | protein |
| Pre | 347 | 141 | 4.4 | 207 | 272 | 62 | 41 |
| Peak | | | | | | | |
| Main | 269 | 111 | 3.5 | 159 | 209 | 60 | 41 |
| Peak | | | | | | | |

Tetranectin-apolipoprotein A-I was expressed in E. coli and purified according to Examples 1 to 3 (protocol 2). After purification, the buffer was exchanged by diafiltration to a solution containing 50 mM Tris, 10 mM L-methionine, 6.7 M guanidinium hydrochloride, pH 7.4. The protein concentration was adjusted to 20.4 mg/ml.

A lipid stock solution was prepared by dissolving 100 moles/l of phospholipid (POPC:DPPC in a ratio 3:1) in a buffer containing 250 mM Tris-HCl, 140 mM NaCl, 10 mM L-methionine, 135 mM sodium cholate, pH 7.4 at room temperature. Refolding buffer was prepared by diluting 3.7 ml of the lipid stock solution into 35.6 ml of 250 mM Tris-HCl, 140 mM NaCl, pH 7.4. This buffer was stirred for an additional 2 hours at room temperature.

Refolding and lipid particle formation was initiated by the addition of 9.8 ml tetranectin-apolipoprotein A-I in 50 mM Tris, 10 mM L-methionine, 6.7 M guanidinium hydrochloride, pH 8.0 to refolding buffer. This results in a 1:5 dilution of the guanidinium hydrochloride. The solution was incubated at room temperature over night while constantly stirring. The removal of the detergent was carried out by diafiltration.

TABLE 24
Summary protein conjugate analysis of lipid particle obtained by
rapid dilution with a POPC/DPPC/cholate mixture.

| | MW | MW | n Protein | MW | | n Lipid/ | |
|---|---|---|---|---|---|---|---|
| Peak | total [kDa] | Protein [kDa] | (APO-Monomer) | Lipid [kDa] | n Lipid | n Protein | % Protein |
| Pre | 419 | 167 | 5.2 | 251 | 333 | 64 | 41 |
| Peak | | | | | | | |
| Main | 252 | 101 | 3.2 | 151 | 200 | 63 | 41 |
| Peak | | | | | | | |

### b) POPC and DPPC and Sodium Cholate

Tetranectin-apolipoprotein A-I was expressed in E. coli and purified according to Examples 1 to 3. After purification, the buffer was exchanged by diafiltration into a solution containing 250 mM Tris, 140 mM NaCl, 6.7 M guanidinium hydrochloride, pH 7.4. The protein concentration was adjusted to 30 mg/ml.

Two separate lipid stock solutions were prepared. Solution A was prepared by dissolving 100 moles/l of POPC in a buffer containing 250 mM Tris-HCl, 140 mM NaCl, 135 mM sodium cholate, pH 7.4 at room temperature. Solution B was prepared by dissolving 100 moles/l of DPPC in 250 mM Tris-HCl, 140 mM NaCl, 135 mM sodium cholate, pH 7.4 at 41° C. Lipid stock solutions A and B were mixed in a ratio of 3:1 and incubated for 2 hours at room temperature. Refolding buffer was prepared by diluting 384 ml of the lipid stock mixture into 6365 ml of 250 mM Tris-HCl, 140 mM NaCl, pH 7.4. This buffer was stirred for an additional 24 hours at room temperature.

Refolding and lipid particle formation was initiated by the addition of 750 ml tetranectin-apolipoprotein A-I solution in 250 mM Tris, 140 mM NaCl, 6.7 M guanidinium hydrochloride, pH 7.4 to the refolding buffer. This results in a 1:10 dilution of the guanidinium hydrochloride. The solution was incubated at room temperature for at least 12 hours while constantly stirring. Detergent removal was carried out by diafiltration.

TABLE 25
Summary protein conjugate analysis of lipid particle obtained by
rapid dilution with POPC:DPPC = 1:1.

| | | MW protein | n (protein | | | n (lipid)/ | |
|---|---|---|---|---|---|---|---|
| Peak | MW total [kDa] | [kDa] | monomer) | MW lipid [kDa] | n (lipid) | n (protein) | % protein |
| Main | 263 | 102 | 3.2 | 161 | 214 | 67 | 39 |
| peak | | | | | | | |
| Post | 182 | 85 | 2.7 | 97 | 129 | 48 | 47 |
| peak | | | | | | | |

### c) Different Guanidinium Hydrochloride Concentrations

Tetranectin-apolipoprotein A-I according to the invention was expressed in E. coli and purified over a metal chelate affinity chromatographic process from inclusion bodies (see Examples 1 to 3). After purification, the buffer was exchanged by diafiltration into a solution containing 250 mM Tris, 140 mM NaCl, 6.7 M guanidinium hydrochloride, pH 7.4. The protein concentration was adjusted to 28 mg/ml.

A lipid stock solution was prepared by dissolving 100 moles/l of POPC in a buffer containing 250 mM Tris-HCl, 140 mM NaCl, 135 mM sodium cholate, pH 7.4 at room temperature. The lipid stock solution was incubated for 2 hours at room temperature. Refolding buffer was prepared by diluting lipid stock solution into 250 mM Tris-HCl, 140 mM NaCl, pH 7.4. This buffer was stirred for an additional 12 hours at room temperature. Varying amounts of tetranectin-apolipoprotein A-I were diluted into refolding buffer: 1:5, 1:7.5, 1:10, 1:12.5. This results in different residual concentrations of guanidinium hydrochloride in the refolding buffer. The solution was allowed to stir at room temperature o/n to initiate refolding and lipid particle formation. Detergent removal was carried out by dialysis.

TABLE 26
Summary protein conjugate analysis of lipid particle obtained by
rapid dilution with different dilution ratios.

| | | | MW protein | n (protein | | | n (lipid)/ | |
|---|---|---|---|---|---|---|---|---|
| dilution | Peak | MW total [kDa] | [kDa] | monomer) | MW lipid [kDa] | n (lipid) | n (protein) | % protein |
| 1:5 | Main | 273 | 103 | 3.2 | 170 | 226 | 70 | 38 |
| 1:7.5 | Main | 272 | 100 | 3.1 | 173 | 230 | 73 | 37 |
| 1:10 | Main | 266 | 106 | 3.3 | 160 | 212 | 64 | 40 |
| 1:12.5 | Main | 281 | 101 | 3.2 | 180 | 239 | 76 | 36 |

### d) POPC and Sodium Cholate in the Presence of Urea

Tetranectin-apolipoprotein A-I is expressed in E. coli and purified according to Examples 1 to 3. After purification, the buffer is exchanged by diafiltration to a solution containing 250 mM Tris, 140 mM NaCl, 6.7 M urea, pH 7.4. The protein concentration is adjusted to 28 mg/ml.

A lipid stock solution is prepared by dissolving 100 moles/l of POPC in a buffer containing 250 mM Tris-HCl, 140 mM NaCl, 135 mM sodium cholate, pH 7.4 at room temperature. The lipid stock solution is incubated for 2 hours at room temperature. Refolding buffer is prepared by diluting 77 ml of the lipid stock mixture into 1478 ml of 250 mM Tris-HCl, 140 mM NaCl, pH 7.4. This buffer is stirred for an additional 7 hours at room temperature.

Refolding and lipid particle formation is initiated by the addition of 162 ml tetranectin-apolipoprotein A-I solution in 250 mM Tris, 140 mM NaCl, 6.7 M urea, pH 7.4 to refolding buffer. This results in a 1:10 dilution of the urea. The solution is incubated at room temperature for 16 hours while constantly stirring. The removal of the detergent is carried out by diafiltration.

### e) POPC and Sodium Cholate and Wild-Type Apolipoprotein A-I

In another exemplary second method human apolipoprotein A-I (wild-type apolipoprotein A-I) in 6.7 M guanidinium hydrochloride, 50 mM Tris, 10 mM methionine, at pH 8.0 was diluted 1:5 (v/v) into lipidation buffer resulting in a protein concentration of 0.6 mg/ml. The lipidation buffer was consisting of 7 mM cholate, 4 mM POPC and 1.3 mM DPPC corresponding to a lipid to protein ratio of 240:1. SEC-MALLS was employed to analyze complex formation. Approximately two apolipoprotein molecules were found in a complex consisting of around 200 lipid molecules.

TABLE 27
Summary of protein conjugate analysis.

| | | | | | | Number | |
|---|---|---|---|---|---|---|---|
| Starting | | MW | MW | n (protein | | of | Ratio |
| material | | total | protein | monomer) | MW lipids | lipids | lipid:protein |
| denatured | Mainpeak | 235 | 71 | 2.2 | 163 | 216 | 1:97 |

### Example 6

### Lipid Particle Formation Starting from Denatured or Native Protein

The method as reported in Example 4 (first method) requires native apolipoprotein for lipid particle formation whereas the method reported in Example 5 (second method) starts with fully denatured apolipoprotein for lipid particle formation.

In an exemplary first method denatured tetranectin-apolipoprotein A-I in 6.7 M guanidinium hydrochloride, 50 mM Tris, 10 mM methionine, at pH 8.0 was extensively dialyzed against a buffer consisting of 250 mM Tris, 140 mM NaCl, 10 mM methionine, at pH 7.5 at a protein concentration of 3.46 mg/ml. A mixture of POPC and cholate was then added to yield a final concentration of 6 mM POPC and 8 mM cholate in the solution. This corresponds to a ratio of 60 molecules of POPC per molecule of tetranectin-apolipoprotein A-I monomer (60:1). The detergent was subsequently removed by diafiltration. Analysis of formed protein-lipid complexes was by SEC-MALLS. Using this method a heterogeneous product was formed wherein approximately 60% of the formed species comprised more than three tetranectin-apolipoprotein A-I monomers.

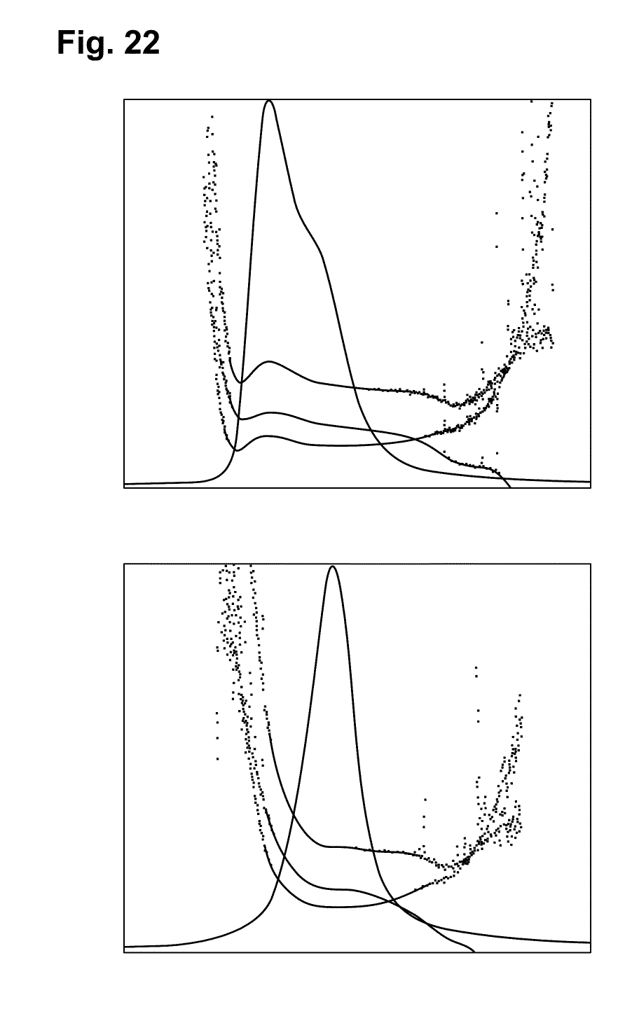

In an exemplary second method denatured tetranectin-apolipoprotein A-I in 6.7 M guanidinium hydrochloride, 50 mM Tris, 10 mM methionine, at pH 8.0 was directly diluted 1:10 (v/v) into lipidation buffer resulting in a protein concentration of 2.5 mg/ml. The lipidation buffer was consisting of 6 mM cholate and 4.5 mM POPC corresponding to a lipid to protein ratio of 60:1. Using this method a homogenous product was formed comprising more than 90% of a single formed species wherein 60 molecules of lipid where bound per molecule of tetranectin-apolipoprotein A-I (see FIG. 22).

TABLE 28
Summary of protein conjugate analysis.

| Starting | | | | n (protein | | Number of | Ratio |
|---|---|---|---|---|---|---|---|
| material | | MW total | MW protein | monomer) | MW lipids | lipids | lipid:protein |
| native | Prepeak (60%) | 321 | 131 | 4.1 | 190 | 250 | 61 |
| | Mainpeak (40%) | 269 | 107 | 3.3 | 162 | 213 | 65 |
| denatured (>90%) | Mainpeak | 269 | 111 | 3.5 | 159 | 209 | 60 |

### Example 7

### Lipidation of Insulin-F with Cholate- and Zwittergent-Solubilized POPC/DPPC

The protein chosen for lipid particle formation is commercially available Insulin (Humalog®, Insulin Lispro, Lilly). The molecular weight of the protein is 5808 Da. To increase the detection limit for insulin in the lipid particle the protein has been labeled with NHS-fluorescein (6-[fluorescein-5(6)-carboxamido]hexanoic acid N-hydroxysuccinimide ester, Sigma Aldrich #46940-5MG-F).

Zwittergent- and cholate-mediated lipidation of NHS-Fluorescein-labeled Insulin (Insulin-F) were carried out as reported in Example 4 using a 1:1 mixture of POPC and DPPC. A 0.5 mM lipid mixture was dissolved in either 1×CMC cholate, 2×CMC Zwittergent 3-8 or 5×CMC Zwittergent 3-10 in PBS pH 7.4. Solubilization of the lipids was achieved at 45° C. for 1 h in an ultrasonic bath. Insulin-F was added to the solubilized lipid at a molar ratio protein:lipid of 1:2 (Zwittergent 3-8) or 1:1.2 (Zwittergent 3-10 and cholate). The lipidation mixtures were incubated for one hour at room temperature followed by extensive dialysis against PBS pH 7.4 to remove the detergent.

The formed lipid particles and control samples were analyzed on SE-HPLC using fluorescence detection (494 nm ext., 521 nm em.) and UV280 absorption. Three different samples per lipidation approach were analyzed on SE-HPLC: Insulin-F dissolved in PBS, liposomes without Insulin F in PBS and lipid particles comprising Insulin-F. Non-lipidated Insulin-F elutes from the column at about 40 min. elution time and the peak is detected by fluorescence and UV280 detection. Lipidated Insulin-F samples elute from the column as two separate peaks detected by fluorescence and UV280. The late peak (peak maximum at approx. 40 min.) co-migrates with the Insulin-F control sample. The early peak at 15 min. elution time has a higher molecular weight then pure Insulin-F and consists of lipidated Insulin-F. Protein free lipid particles elute at 15 min. elution time.

### Example 8

### Application of Apolipoprotein

### a) Impact of DPPC and POPC on LCAT Activity

Lipid particles comprising either palmitoyl oleoyl phosphatidylcholine (POPC) or dipalmitoyl phosphatidylcholine (DPPC) and either recombinant wild-type apolipoprotein A-I or tetranectin-apolipoprotein A-I were examined for their ability to support cholesterol esterification by LCAT.

Tritiated cholesterol (4%; relative to the phosphatidylcholine content on a molar basis) was incorporated in the lipid particle by addition of an ethanolic cholesterol solution. The capacity of the resulting protein-lipid complex to support LCAT catalyzed cholesterol esterification was tested in presence of 0.2 μg/ml recombinant LCAT enzyme (ROAR biochemical) in 125 μl (10 mM Tris, 150 mM NaCl, 1 mM EDTA, 1 mM NaN₃; pH 7.4; 2 mg/ml HuFAF Albumin; 4 mM Beta mercaptoethanol) for 1 hour at 37° C. The reaction was stopped by addition of chloroform:methanol (2:1) and lipids were extracted. “Percent” esterification was calculated after cholesterol-cholesteryl ester separation by TLC and scintillation counting. As less than 20% of the tracer was incorporated into the formed ester, the reaction rate could be considered constant under the experimental conditions. Data were fitted to the Michaelis Menten equation using XLfit software (IDBS). For a visualization of the results see FIG. 3.

### b) Impact of DPPC/POPC Mixtures on LCAT Activity

Lipid particles were prepared using cholate as detergent by mixing recombinant wild-type apolipoprotein A-I with ³H cholesterol, a DPPC/POPC mixture, and cholate in 1:4:80:113 molar ratios. DPPC/POPC mixtures contained either 100% POPC; 75% POPC; 50% POPC; 25% POPC.

After cholate removal by dialysis, the capacity of the resulting protein-lipid complex to support LCAT catalyzed cholesterol esterification was tested. ³H cholesterol (4%; relative to the phosphatidylcholine content on a molar basis) was incorporated in the lipid particle by addition of an ethanolic cholesterol solution. The capacity of the resulting protein-lipid complex to support LCAT catalyzed cholesterol esterification was tested in presence of 0.2 μg/ml recombinant LCAT enzyme (ROAR biochemical) in 125 μl (10 mM Tris, 150 mM NaCl, 1 mM EDTA, 1 mM NaN₃; pH 7.4; 2 mg/ml HuFAF Albumin; 4 mM beta mercaptoethanol) for 1 hour at 37° C. The reaction was stopped by addition of chloroform:methanol (2:1) and lipids were extracted. “Percent” esterification was calculated after cholesterol-cholesteryl ester separation by TLC and scintillation counting. As less than 20% of the tracer was incorporated into esters, the reaction rate could be considered as constant in the experimental conditions. Data were fitted to the Michaelis Menten equation using XLfit software (IDBS) and are shown in FIG. 4.

TABLE 3a
Apparent kinetic parameters.

| substrate | Kₘ | Vₘₐₓ |
|---|---|---|
| [% POPC] | [nM] | [n mole ester/h/U LCAT] |
| 100 | 4.6 | 1.6 |
| 75 | 0.4 | 1.9 |
| 50 | 0.5 | 1.8 |
| 25 | 1.0 | 1.7 |
| 0 | 6.9 | 1.8 |

### c) Cholesterol Efflux to THP-1 Derived Foam Cells

Macrophage like human THP-1 cells, were obtained by exposing THP-1 monocytic leukemia cells to phorbol myristate acetate. Subsequently cells were loaded by further culture in the presence of acetylated LDL containing ³H Cholesterol tracer. These model foam cells were then exposed for 4 h-8 h to cholesterol acceptor test compounds (see below).

Cell culture supernatants were harvested and cells lysed in 5% NP40. Fractional efflux was calculated as the ratio of cholesterol radioactivity in the supernatant relative to the sum of the radioactivity in the cells plus supernatant. Efflux from cell exposed to medium containing no acceptors was subtracted and efflux velocity calculated by linear fit. Efflux velocity was standardized using efflux from cells to 10 μg/ml wild-type apolipoprotein A-I as reference (relative efflux velocity). Relative efflux velocities obtained in two separate experiments were plotted as function of cholesterol acceptor concentration and data fitted to the Michaelis Menten equation.

Parallel experiments were performed using cells exposed to a RXR-LXR agonist that is known to upregulate ABCA-1 transporters, and bias cholesterol transport toward ABCA-1 mediated efflux.

Only a modest influence of the lipid mixture was observed in the tested series (FIG. 5 and Table 29).

TABLE 29
Different samples.

| | molar ratio | |
|---|---|---|
| tetranectin- | apolipo- | |
| apolipoprotein | protein:phos- | preparation |
| A-I with | pholipid | method |
| 100% POPC/ | 1:60 | cholate |
| 0% DPPC | | |
| 75% POPC/ | 1:60 | cholate |
| 25% DPPC | | |
| 50% POPC/ | 1:70 | cholate |
| 50% DPPC | | |
| 0% POPC/ | 1:80 | cholate |
| 100% DPPC | | |
| — | not | |

RXR-LXR pretreatment of the foam cells strongly increased efflux to the non-lipidated material with a six-fold increase of the maximal velocity over non treated cells. Impact on lipid particles was much less, with a two-fold increase, reflecting lower contribution of the ABCA-1 transporter to the cholesterol efflux (FIG. 6).

### d) In Vivo Study

Five lipid particle variants were studied:
i) only POPC
ii) only DPPC
iii) POPC:DPPC 3:1
iv) POPC:DPPC 1:1
v) DPPC:SM 9:1

Rabbits were intravenous infused over 0.5 h at 80 mg/kg (n=3 rabbits/test compound) followed by serial blood sampling over 96 h post infusion.

Analysis of apolipoprotein levels with an ELISA:
drug levels
data on plasma values of liver enzymes, cholesterol, cholesterol ester.

Plasma concentrations are very similar for all tested compositions showing little pronounced initial “distribution” phase followed by log-linear decline of concentrations (FIG. 7, Table 3).

TABLE 3
Pharmacokinetic data.

| tetranectin- | | | | |
|---|---|---|---|---|
| apolipoprotein | C_{L} | vₛₛ | T_{1/2} | Cₘₐₓ |
| A-I with | [ml/h/kg] | [ml/kg] | [h] | [mg/m] |
| 100% POPC/ | 0.897 ± 0.216 | 45.0 ± 2.5 | 36.9 ± 8.2 | 2.40 ± 0.19 |
| 0% DPPC | | | | |
| 0% POPC/ | 0.922 ± 0.098 | 37.8 ± 4.9 | 30.2 ± 7.7 | 2.29 ± 0.19 |
| 100% DPPC | | | | |
| 75% POPC/ | 0.815 ± 0.064 | 37.8 ± 5.6 | 34.2 ± 4.5 | 2.65 ± 0.28 |
| 25% DPPC | | | | |
| 50% POPC/ | 0.850 ± 0.135 | 43.1 ± 5.9 | 38.6 ± 10.6 | 2.34 ± 0.31 |
| 50% DPPC | | | | |
| 90% DPPC/ | 1.28 ± 0.62 | 50.7 ± 8.7 | 31.3 ± 8.2 | 1.91 ± 0.33 |
| 10% SM | | | | |

The determined pharmacokinetic (PK) parameters were similar for all tested compounds. Also a low inter-individual variability has been found. The determined half-lives are close to 1.5 days, i.e. increased compared to wild-type apolipoprotein A-I. The volume of distribution is similar to plasma volume (ca. 40 ml/kg in rabbits).

### f) Cholesterol Mobilization

Cholesterol is mobilized and esterified in plasma. Plasma cholesteryl ester levels do continue to increase even after tetranectin-apolipoprotein A-I is already decreasing. When plasma tetranectin-apolipoprotein A-I levels have decreased to 0.5 mg/ml (about 50% of normal wild-type apolipoprotein A-I) increased cholesterol ester levels are still detectable (FIG. 8).

### g) Liver Enzyme Release

Lipid particles comprising tetranectin-apolipoprotein A-I containing POPC do not induce liver enzyme release (FIG. 1). Similar to the rabbit, a single i.v. injection of the tetranectin-apolipoprotein A-I according to the current invention containing POPC or POPC/DPPC mixtures are safe in mice. The apolipoprotein composition containing DPPC:POPC at a molar ratio of 1:3 was comparable to POPC alone (FIG. 9).

No significant hemolysis was observed until two hours post infusion in any of the five preparations. Hemolysis was determined photometrically as red color in plasma samples obtained at two hours after i.v. application of tetranectin-apolipoprotein A-I. 100% hemolysis of whole blood (generated by 0.44% Triton X-100-final concentration) was used for calibration (FIG. 10).

### h) Anti-Inflammatory Effects of Tetranectin-Apolipoprotein A-I on Human Umbilical Vein Endothelial Cells

Passage 5-10 HUVECs (human umbilical vein endothelial cells) were incubated in the respective tetranectin-apolipoprotein A-I preparations for 16 h and stimulated with TNFα for the final 4 hours. VCAM1 surface expression was detected with specific antibodies by FACS.

### Example 9

### Lipid Particle Stability

Wild-type Apolipoprotein A-I containing an N-terminal histidine-tag and an IgA protease cleavage site was expressed in E. coli and purified by column chromatography as reported in the examples above. The histidine-tag was removed by IgA protease cleavage. Lipid particles (HDL particles) were assembled using a 1:150 ratio of protein to Lipoid 5100 soybean phospholipid mixture. The particles were stored in a buffer containing 5 mM sodium phosphate and 1% sucrose at pH value of 7.3. SE-HPLC revealed three distinct peaks upon incubation after lipidation and incubation for 10 days. After incubation at 40° C., a predominant peak at retention time 10.8 minutes can be detected (47% of total protein), which is absent in the sample stored at 5° C. The 10.8 minutes peak indicates the formation of soluble large molecular weight assemblies due to protein destabilization.

HDL particles containing tetranectin-apolipoprotein A-I as reported herein which were obtained starting from a POPC:DPPC mixture (ratio POPC to DPPC of 3:1) were also incubated at 5° C. and 40° C. Incubation at elevated temperature lead to a slight degree of pre-peak formation, but no significant shift to high molecular weight assemblies at 10.8 minutes (<2% increase at 11 minutes). This indicates improved HDL particle stability compared to the particle containing wild-type apolipoprotein A-I.

### Example 10

### Cholesterol Mobilization

The efficiency at which cholesterol is mobilized into the blood can be shown by monitoring the ratio of cholesterol concentration in the blood to apolipoprotein concentration in the blood, especially when the ratio of the AUC values (area under the curve) of these parameters determined in vivo time dependent after application is taken.

In this experiment the following substances were analyzed:
wild-type apolipoprotein A-I containing an N-terminal histidine-tag and an IgA protease cleavage site expressed in E. coli and purified by column chromatography as reported in the examples above; the histidine-tag was removed by IgA protease cleavage; lipid particles (HDL particles) were assembled using a 1:150 ratio of protein to Lipoid S100 soybean phospholipid mixture,
apolipoprotein A-I Milano variant; lipid particles (HDL particles) were assembled using a 1:40 ratio of protein to POPC,
tetranectin-apolipoprotein A-I as reported herein; lipid particles (HDL particles) were assembled using a 1:60 ratio of protein to POPC and DPPC (POPC and DPPC at a ratio of 3:1).

The three HDL particles were applied to rats. The values obtained for the respective AUC ratios are shown in Table 30.

TABLE 30
Cholesterol mobilization.

| | | AUC(time dependent concentra- |
|---|---|---|
| | | tion cholesterol in blood) |
| | | AUC (time dependent apolipo- |
| | lipids | protein A-I concentration in blood) |
| wt-apolipoprotein | soybean | 0.0002 (mmol/l)/(μg/ml)). |
| A-I | phospholipid | |
| | mixture | |
| apolipoprotein A-I | POPC | 0.0004 (mmol/l)/(μg/ml)). |
| Milano variant | | |
| tetranectin- | POPC:DPPC | 0.0013 (mmol/l)/(μg/ml) |
| apolipoprotein A-I | 3:1 | |
| as reported herein | | |

1-28. (canceled)

29. A method for producing a lipid particle comprising a polypeptide, characterized in that the lipid particle is formed in the presence of a synthetic detergent comprising Zwittergent 3-8 or Zwittergent 3-10.

30. The method according to claim 1, characterized in comprising:
i) providing a first solution comprising denatured polypeptide,
ii) adding the first solution to a second solution comprising at least one lipid and the synthetic detergent but which is free of the polypeptide, and
iii) removing the synthetic detergent from the solution obtained in ii) and thereby producing a lipid particle.

31. The method according to claim 29, characterized in comprising:
i) providing a solution comprising native polypeptide,
ii) adding a lipid and and the synthetic detergent to the solution of i), and
iii) removing the synthetic detergent from the solution obtained in ii) and thereby producing a lipid particle.

32. The method according to claim 30, characterized in that the polypeptide has an amino acid sequence selected from the amino acid sequences of SEQ ID NO: 01, 02, 04 to 52, 66, or 67, or comprises at least a contiguous fragment comprising at least 80% of the amino acid sequence of SEQ ID NO: 01, 02, 04 to 52, 66, or 67.

33. The method according to claim 32, characterized in that the polypeptide is a tetranectin-apolipoprotein A-I that has the amino acid sequence of SEQ ID NO: 01 or SEQ ID NO: 02 or SEQ ID NO: 66 or SEQ ID NO: 67.

34. The method according to claim 30, characterized in that the at least one lipid is two different phosphatidylcholines.

35. The method according to claim 34, characterized in that the first phosphatidylcholine is POPC and the second phosphatidylcholine is DPPC.

36. The method according to claim 30, characterized in that the method comprises after ii) and prior to iii) the following:
iia) incubating the solution obtained in ii).

37. The method according to claim 36, characterized in that the incubating is for about 0.5 hours to about 20 hours.

38. The method according to claim 30, characterized in that the detergent is a detergent with a high CMC.

39. The method according to claim 30, characterized in that the removing is by diafiltration or dialysis or adsorption.

40. A lipid particle obtained with a method according to claim 30.

41. A lipid particle obtained with a method according to claim 31.

42. A pharmaceutical composition comprising a lipid particle according to claim 30.

43. A pharmaceutical composition comprising a lipid particle according to claim 31.

44. A method for producing a lipid particle comprising:
i) providing a first solution comprising a denatured protein,
ii) adding the first solution to a second solution comprising at least one lipid and a detergent but not the protein, and
iii) removing the detergent from the solution obtained in ii) and thereby producing a lipid particle.

45. The method according to claim 44 characterized in that the protein has an amino acid sequence selected from the amino acid sequences of SEQ ID NO: 01, 02, 04 to 52, 66, or 67, or comprises at least a contiguous fragment comprising at least 80% of the amino acid sequence of SEQ ID NO: 01, 02, 04 to 52, 66, or 67.

46. The method according to claim 45, characterized in that the protein is a tetranectin-apolipoprotein A-I that has the amino acid sequence of SEQ ID NO: 01, or SEQ ID NO: 02, or SEQ ID NO: 66, or SEQ ID NO: 67.

47. The method according to claim 44, characterized in that the at least one lipid is two different phosphatidylcholines.

48. The method according to claim 47, characterized in that the first phosphatidylcholine is POPC and the second phosphatidylcholine is DPPC.

49. The method according to claim 44, characterized in that the detergent is selected from cholic acid, Zwittergent or a salt thereof.

50. The method according to claim 29, characterized in that the method comprises after ii) and prior to iii) the following step
iia) incubating the solution obtained in step ii).

51. The method according to claim 29, characterized in that the incubating and/or removing is at a temperature of from 4° C. to 45° C.

52. The method according to claim 49, characterized in that the incubating is for about 0.5 hours to about 20 hours.

53. The method according to claim 29, characterized in that the detergent is a detergent with a high CMC.

54. The method according to claim 29, characterized in that the removing is by diafiltration or dialysis or adsorption.

55. A lipid particle obtained with a method according to claim 29.

56. A pharmaceutical composition comprising a lipid particle according to claim 29.


SEQUENCE LISTING (105 sequences)

SEQ ID NO: 1 - protein, 285 aa, Artificial Sequence
  Description of Artificial Sequence Synthetic Tetranectin-apolipoprotein A-I (1) polypeptide
APIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 283 aa, Artificial Sequence
  Description of Artificial Sequence Synthetic Tetranectin-apolipoprotein A-I (2) polypeptide
IVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 5 aa, Homo sapiens
SLKGS

SEQ ID NO: 4 - protein, 22 aa, Artificial Sequence
  Description of Artificial Sequence Synthetic Apolipoprotein A-I mimetic (1) peptide
PVLDEFREKLNEELEALKQKLK

SEQ ID NO: 5 - protein, 22 aa, Artificial Sequence
  Description of Artificial Sequence Synthetic Apolipoprotein A-I mimetic (2) peptide
PVLDLFRELLNELLEALKQKLK

SEQ ID NO: 6 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 7 - protein, 100 aa, Homo sapiens
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 8 - protein, 396 aa, Homo sapiens [sequence omitted]

SEQ ID NO: 9 - protein, 366 aa, Homo sapiens
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 10 - protein, 83 aa, Homo sapiens
MRLFLSLPVLVVVLSIVLEGPAPAQGTPDVSSALDKLKEFGNTLEDKARELISRIKQSELSAKMREWFSETFQKVKEKLKIDS

SEQ ID NO: 11 - protein, 101 aa, Homo sapiens
MGTRLLPALFLVLLVLGFEVQGTQQPQQDEMPSPTFLTQVKESLSSYWESAKTAAQNLYEKTYLPAVDEKLRDLYSKSTAAMSTYTGIFTDQVLSVLKGEE

SEQ ID NO: 12 - protein, 99 aa, Homo sapiens
MQPRVLLVVALLALLASARASEAEDASLLSFMQGYMKHATKTAKDALSSVQESQVAQQARGWVTDGFSSLKDYWSTVKDKFSEFWDLDPEVRPTSAVAA

SEQ ID NO: 13 - protein, 127 aa, Homo sapiens
MSLLRNRLQALPALCLCVLVLACIGACQPEAQEGTLSPPPKLKMSRWSLVRGRMKELLETVVNRTRDGWQWFWSPSTFRGFMQTYYDDHLRDLGPLTKAWFLESKDSLLKKTHSLCPRLVCGDKDQG

SEQ ID NO: 14 - protein, 189 aa, Homo sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 15 - protein, 317 aa, Homo sapiens [sequence omitted]

SEQ ID NO: 16 - protein, 308 aa, Homo sapiens [sequence omitted]

SEQ ID NO: 17 - protein, 345 aa, Homo sapiens
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 18 - protein, 398 aa, Homo sapiens
MEGAALLRVSVLCIWMSALFLGVGVRAEEAGARVQQNVPSGTDTGDPQSKPLGDWAAGTMDPESSIFIEDAIKYFKEKVSTQNLLLLLTDNEAWNGFVAAAELPRNEADELRKALDNLARQMIMKDKNWHDKGQQYRNWFLKEFPRLKSELEDNIRRLRALADGVQKVHKGTTIANVVSGSLSISSGILTLVGMGLAPFTEGGSLVLLEPGMELGITAALTGITSSTMDYGKKWWTQAQAHDLVIKSLDKLKEVREFLGENISNFLSLAGNTYQLTRGIGKDIRALRRARANLQSVPHASASRPRVTEPISAESGEQVERVNEPSILEMSRGVKLTDVAPVSFFLVLDVVYLVYESKHLHEGAKSETAEELKKVAQELEEKLNILNNNYKILQADQEL

SEQ ID NO: 19 - protein, 337 aa, Homo sapiens
MNPESSIFIEDYLKYFQDQVSRENLLQLLTDDEAWNGFVAAAELPRDEADELRKALNKLASHMVMKDKNRHDKDQQHRQWFLKEFPRLKRELEDHIRKLRALAEEVEQVHRGTTIANVVSNSVGTTSGILTLLGLGLAPFTEGISFVLLDTGMGLGAAAAVAGITCSVVELVNKLRARAQARNLDQSGTNVAKVMKEFVGGNTPNVLTLVDNWYQVTQGIGRNIRAIRRARANPQLGAYAPPPHIIGRISAEGGEQVERVVEGPAQAMSRGTMIVGAATGGILLLLDVVSLAYESKHLLEGAKSESAEELKKRAQELEGKLNFLTKIHEMLQPGQDQ

SEQ ID NO: 20 - protein, 402 aa, Homo sapiens
MGLGQGWGWEASCFACLIRSCCQVVTFTFPFGFQGISQSLENVSGYYADARLEVGSTQLRTAGSCSHSFKRSFLEKKRFTEEATKYFRERVSPVHLQILLTNNEAWKRFVTAAELPRDEADALYEALKKLRTYAAIEDEYVQQKDEQFREWFLKEFPQVKRKIQESIEKLRALANGIEEVHRGCTISNVVSSSTGAASGIMSLAGLVLAPFTAGTSLALTAAGVGLGAASAVTGITTSIVEHSYTSSAEAEASRLTATSIDRLKVFKEVMRDITPNLLSLLNNYYEATQTIGSEIRAIRQARARARLPVTTWRISAGSGGQAERTIAGTTRAVSRGARILSATTSGIFLALDVVNLVYESKHLHEGAKSASAEELRRQAQELEENLMELTQIYQRLNPCHTH

SEQ ID NO: 21 - protein, 351 aa, Homo sapiens
MEGAALLKIFVVCIWVQQNHPGWTVAGQFQEKKRFTEEVIEYFQKKVSPVHLKILLTSDEAWKRFVRVAELPREEADALYEALKNLTPYVAIEDKDMQQKEQQFREWFLKEFPQIRWKIQESIERLRVIANEIEKVHRGCVIANVVSGSTGILSVIGVMLAPFTAGLSLSITAAGVGLGIASATAGIASSIVENTYTRSAELTASRLTATSTDQLEALRDILRDITPNVLSFALDFDEATKMIANDVHTLRRSKATVGRPLIAWRYVPINVVETLRTRGAPTRIVRKVARNLGKATSGVLVVLDVVNLVQDSLDLHKGAKSESAESLRQWAQELEENLNELTHIHQSLKAG

SEQ ID NO: 22 - protein, 433 aa, Homo sapiens
MPCGKQGNLQVPGSKVLPGLGEGCKEMWLRKVIYGGEVWGKSPEPEFPSLVNLCQSWKINNLMSTVHSDEAGMLSYFLFEELMRCDKDSMPDGNLSEEEKLFLSYFPLHKFELEQNIKELNTLADQVDTTHELLTKTSLVASSSGAVSGVMNILGLALAPVTAGGSLMLSATGTGLGAAAAITNIVTNVLENRSNSAARDKASRLGPLTTSHEAFGGINWSEIEAAGFCVNKCVKAIQGIKDLHAYQMAKSNSGFMAMVKNFVAKRHIPFWTARGVQRAFEGTTLAMTNGAWVMGAAGAGFLLMKDMSSFLQSWKHLEDGARTETAEELRALAKKLEQELDRLTQHHRHLPQKASQTCSSSRGRAVRGSRVVKPEGSRSPLPWPVVEHQPRLGPGVALRTPKRTVSAPRMLGHQPAPPAPARKGRQAPGRHRQ

SEQ ID NO: 23 - protein, 343 aa, Homo sapiens
MDNQAERESEAGVGLQRDEDDAPLCEDVELQDGDLSPEEKIFLREFPRLKEDLKGNIDKLRALADDIDKTHKKFTKANMVATSTAVISGVMSLLGLALAPATGGGSLLLSTAGQGLATAAGVTSIVSGTLERSKNKEAQARAEDILPTYDQEDREDEEEKADYVTAAGKIIYNLRNTLKYAKKNVRAFWKLRANPRLANATKRLLTTGQVSSRSRVQVQKAFAGTTLAMTKNARVLGGVMSAFSLGYDLATLSKEWKHLKEGARTKFAEELRAKALELERKLTELTQLYKSLQQKVRSRARGVGKDLTGTCETEAYWKELREHVWMWLWLCVCLCVCVYVQFT

SEQ ID NO: 24 - protein, 188 aa, Homo sapiens
MFHQIWAALLYFYGIILNSIYQCPEHSQLTTLGVDGKEFPEVHLGQWYFIAGAAPTKEELATFDPVDNIVFNMAAGSAPMQLHLRATIRMKDGLCVPRKWIYHLTEGSTDLRTEGRPDMKTELFSSSCPGGIMLNETGQGYQRFLLYNRSPHPPEKCVEEFKSLTSCLDSKAFLLTPRNQEACELSNN

SEQ ID NO: 25 - protein, 198 aa, Homo sapiens
MFKVIQRSVGPASLSLLTFKVYAAPKKDSPPKNSVKVDELSLYSVPEGQSKYVEEARSQLEESISQLRHYCEPYTTWCQETYSQTKPKMQSLVQWGLDSYDYLQNAPPGFFPRLGVIGFAGLIGLLLARGSKIKKLVYPPGFMGLAASLYYPQQAIVFAQVSGERLYDWGLRGYIVIEDLWKENFQKPGNVKNSPGTK

SEQ ID NO: 26 - protein, 268 aa, Homo sapiens
MAAIRMGKLTTMPAGLIYASVSVHAAKQEESKKQLVKPEQLPIYTAPPLQSKYVEEQPGHLQMGFASIRTATGCYIGWCKGVYVFVKNGIMDTVQFGKDAYVYLKNPPRDFLPKMGVITVSGLAGLVSARKGSKFKKITYPLGLATLGATVCYPVQSVIIAKVTAKKVYATSQQIFGAVKSLWTKSSKEESLPKPKEKTKLGSSSEIEVPAKTTHVLKHSVPLPTELSSEAKTKSESTSGATQFMPDPKLMDHGQSHPEDIDMYSTRS

SEQ ID NO: 27 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 28 - protein, 267 aa, Homo sapiens; identical to SEQ ID NO: 6

SEQ ID NO: 29 - protein, 267 aa, Homo sapiens; identical to SEQ ID NO: 6

SEQ ID NO: 30 - protein, 267 aa, Homo sapiens
MKATVLTLAVLFLTGSQARHFWQQDEPPQTPWDRVKDLVTVYVEALKDSGKDYVSQFEGSALGKQLNLKLLDNWDSVTSTVSKLREQLGPVTQEFWDNLEKETEGLRQEMSKDLEEVKAKVQPYLDDFQKKWQEEMELYRQKVEPLRAELHEGTRQKLHELHEKLSPLGEEVRDRARAHVDALRTHLAPYSDELRQRLAARLEALKENGGARLAEYHAKASEHLSTLSEKAKPALEDLRQGLLPVLESFKVSFLSALEEYTKKLSTQ

SEQ ID NO: 31 - protein, 265 aa, Homo sapiens
MKAVVLTLAVLFLTGSQARHFWQQDDPQSSWDRVKDFATVYVEAIKDSGRDYVAQFEASALGKQLNLKLLDNWDTLASTLSKVREQLGPVTQEFWDNLEKETASLRQEMHKDLEEVKQKVQPYLDEFQKKWHEEVEIYRQKVAPLGEEFREGARQKVQELQDKLSPLAQELRDRARAHVETLRQQLAPYSDDLRQRLTARLEALKEGGGSLAEYHAKASEQLKALGEKAKPVLEDLRQGLLPVLESLKVSILAAIDEASKKLNAQ

SEQ ID NO: 32 - protein, 264 aa, Homo sapiens
MKAWLTLAVLFLTGSQARHFWQQDDPQSPWDRVKDFATVYVDAIKDSGRDYVAQFEASALGKHLNLKLLDNWDSLGSTFTKVREQLGPVTQEFWDNLEKETEALRQEMSKDLEEVKKKVQPYLDDFQNKWQEEMETYRQKMAPLGAEFREGARQKVQELQEKLSPLAEELRDRLRAHVEALRQHVAPYSDDLRQRMAARFEALKEGGGSLAEYQAKAQEQLKALGEKAKPALEDLRQGLLPVLENLKVSILAAIDEASKKLNAQ

SEQ ID NO: 33 - protein, 266 aa, Homo sapiens
MKAALLTLAVLFLTGSQARHFWQQDEPQSPWDRVKDLATVYVDAVKDSGRDYVAQFEASALGKQLNLKLLDNWDSLSSTVTKLREQIGPVTQEFWDNLEKETEVLRQEMSKDLEEVKQKVQPYLDDFQKKWQEEVELYRQKVAPLGSELREGARQKLQELQEKLSPLAEELRDRARTHVDALRAQLAPYSDDLRERLAARLEALKEGGGASLAEYHARASEQLSALGEKARPALEDLRQGLLPVLESFKVSLLAAIDEATKKLNAQ

SEQ ID NO: 34 - protein, 206 aa, Homo sapiens
MKAVVLTLAVLFLTGSQARHFWQRDEPRSSWDKIKDFATVYVDTVKDSGREYVAQFEASAFGKQLNLKLLDNWDSLSSTVSKLQEQLGPVTQEFWDNLEKETEGLREEMNKDLQEVRQKVQPYLDEFQKKWQEEVERYRQKVEPLGAELRESARQKLTELQEKLSPLAEELRDSARTHVGLLPVLESFKASVQNVLDEATKKLNTQ

SEQ ID NO: 35 - protein, 265 aa, Homo sapiens
MKAVVLTLAVLFLTGSQARHFWQQDEPQSSWDRVRDLANVYVDAVKESGREYVSQLEASALGKQLNLKLVDNWDTLGSTFQKVHEHLGPVAQEFWEKLEKETEELRREINKDLEDVRQKTQPFLDEIQKKWQEDLERYRQKVEPLSAQLREGARQKLMELQEQVTPLGEDLRDSVRAYADTLRTQLAPYSEQMRKTLGARLEAIKEGGSASLAEYHAKASEQLSALGEKAKPVLEDIHQGLMPMWESFKTGVLNVIDEAAKKLTA

SEQ ID NO: 36 - protein, 264 aa, Homo sapiens
MKAVVLAVALVFLTGSQAWHVWQQDEPQSQWDKVKDFANVYVDAVKDSGRDYVSQFESSSLGQQLNLNLLENWDTLGSTVSQLQERLGPLTRDFWDNLEKETDWVRQEMNKDLEEVKQKVQPYLDEFQKKWKEDVELYRQKVAPLGAELQESARQKLQELQGRLSPVAEEFRDRMRTHVDSLRTQLAPHSEQMRESLAQRLAELKSNPTLNEYHTRAKTHLKTLGEKARPALEDLRHSLMPMLETLKTKAQSVIDKASETLTAQ

SEQ ID NO: 37 - protein, 259 aa, Homo sapiens
MKAAVLAVALVFLTGCQAWEFWQQDEPQSQWDRVKDFATVYVDAVKDSGRDYVSQFESSTLGKQLNLNLLDNWDTLGSTVGRLQEQLGPVTQEFWANLEKETDWLRNEMNKDLENVKQKMQPHLDEFQEKWNEEVEAYRQKLEPLGTELHKNAKEMQRHLKVVAEEFRDRMRVNADALRAKFGLYSDQMRENLAQRLTEIRNHPTLIEYHTKAGDHLRTLGEKAKPALDDLGQGLMPVLEAWKAKIMSMIDEAKKKLNA

SEQ ID NO: 38 - protein, 241 aa, Homo sapiens
DEAKSYWDQIKDMLTVYVDTAKDSGKDYLTSLDTSALGQQLNKKLADNWDTVSSALLKAREQMKPIAMEFWGNLEKDTEGLRQTVSKDLELVKEKVQPYLDSFQKKVEEELELYRQKVAPLSAEWREQARQKAQELQQKAGELGQQHRDRVRTHVDALRTDLAPYGEEARKLLLQRLQDIKAKSGDLAEYQTKLSEHLKSFGEKAQPTLQDLRHGLEPLWEGIKAGAMSMLEELGKKLNSQ

SEQ ID NO: 39 - protein, 264 aa, Homo sapiens
MRGVLVTLAVLFLTGTQARSFWQHDEPQTPLDRIRDMVDVYLETVKASGKDAIAQFESSAVGKQLDLKLADNLDTLSAAAAKLREDMAPYYKEVREMWLKDTEALRAELTKDLEEVKEKIRPFLDQFSAKWTEELEQYRQRLTPVAQELKELTKQKVELMQAKLTPVAEEARDRLRGHVEELRKNLAPYSDELRQKLSQKLEEIREKGIPQASEYQAKVMEQLSNLREKMTPLVQEFRERLTPYAENLKNRLISFLDELQKSVA

SEQ ID NO: 40 - protein, 264 aa, Homo sapiens
MRGVLVTLAVLFLTGTQARSFWQHDDPQTPLDRIRDMLDVYLETVKASGKDAISQFESSAVGKQLDLKLADNLDTLSAAAAKLREDMTPYYREVREMWLKDTEALRAELTKDLEEVKEKIRPFLDQFSAKWTEEVEQYRQRLAPVAQELKDLTKQKVELMQAKLTPVAEEVRDRLREQVEELRKNLAPYSSELRQKLSQKLEEIRERGIPQASEYQAKVVEQLSNLREKMTPLVQEFKERLTPYAENLKNRLIDLLDEVQKTMA

SEQ ID NO: 41 - protein, 264 aa, Homo sapiens
MRVVVVTLALLFLTGTQARYFWQHDEPQAPLDRLRDLVDVYLETVKASGKDAIAQFEASAVGKQLDLKLADNLDTLGAAAAKLREDMAPYYKEVREMWLKDTESLRAELTKDLEEVKEKIRPFLDQFSAKWTEELEQYRQRLAPVAEELKELTKQKVELMQQKLTPVAEEARDRLRGHVEELRKNLAPYSDELRQKLSQKLEEIREKGIPQAAEYQAKVVEQLSNLREKMTPLVQDFKERLTPYAENLKTRFISLLDELQKTVA

SEQ ID NO: 42 - protein, 262 aa, Homo sapiens
MKFLALALTILLAAGTQAFPMQADAPSQLEHVKAALSMYIAQVKLTAQRSIDLLDDTEYKEYKMQLTQSLDNLQQYADATSQSLAPYSEAFGTQLTDATAAVRAEVMKDVEELRSQLEPKRAELKEVLDKHIDEYRKKLEPLIKEHIELRRTEMEAFRAKMEPIVEELRAKVAINVEETKTKLMPIVEIVRAKLTERLEELRTLAAPYAEEYKEQMIKAVGEVREKVSPLSEDFKGQVGPAAEQAKQKLLAFYETISQAMKA

SEQ ID NO: 43 - protein, 262 aa, Homo sapiens
MKFLALALTILLAAATQAVPMQADAPSQLEHVKVAMMEYMAQVKETGQRSIDLLDDTEFKEYKVQLSQSLDNLQQYAQTTSQSLAPYSEAFGAQLTDAAAAVRAEVMKDVEDVRTQLEPKRAELKEVLDKHIDEYRKKLEPLIKEIVEQRRTELEAFRVKMEPVVEEMRAKVSTNVEETKAKLMPIVETVRAKLTERLEELRTLAAPYAEEYKEQMFKAVGEVREKVGPLTNDFKGQVGPAAEQAKEKLMDFYETISQAMKA

SEQ ID NO: 44 - protein, 258 aa, Homo sapiens
MKFLVLALTILLAAGTQAFPMQADAPSQLEHVKAALNMYIAQVKLTAQRSIDLLDDTEYKEYKMQLSQSLDNLQQFADSTSKSWPPTPRSSAPSCDATATVRAEVMKDVEDVRTQLEPKRAELTEVLNKHIDEYRKKLEPLIKQHIELRRTEMDAFRAKIDPVVEEMRAKVAVNVEETKTKLMPIVEIVRAKLTERLEELRTLAAPYAEEYKEQMFKAVGEVREKVAPLSEDFKARWAPPPRRPSKSSWLSTRPSARP

SEQ ID NO: 45 - protein, 262 aa, Homo sapiens
MKFVALALTLLLALGSQANLFQADAPTQLEHYKAAALVYLNQVKDQAEKALDNLDGTDYEQYKLQLSESLTKLQEYAQTTSQALTPYAETISTQLMENTKQLRERVMTDVEDLRSKLEPHRAELYTALQKHIDEYREKLEPVFQEYSALNRQNAEQLRAKLEPLMDDIRKAFESNIEETKSKVVPMVEAVRTKLTERLEDLRTMAAPYAEEYKEQLVKAVEEAREKIAPHTQDLQTRMEPYMENVRTTFAQMYETIAKAIQA

SEQ ID NO: 46 - protein, 260 aa, Homo sapiens
MKFAALALALLLAVGSHAASMQADAPSQLDHARAVLDVYLTQVKDMSLRAVNQLDDPQYAEFKTNLAQRIEEMYTQIKTLQGSVSPMTDSFYNTVMEVTKDTRESLNVDLEALKSSLAPQNEQLKQVIEKHLNDYRTLLTPIYNDYKTKHDEEMAALKTRLEPVMEELRTKIQANVEETKAVLMPMVETVRTKVTERLESLREVVQPYVQEYKEQMKQMYDQAQTVDTDALRTKITPLVEEIKVKMNAIFEIIAASVTKS

SEQ ID NO: 47 - protein, 396 aa, Homo sapiens
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVNTQAEQLRRQLTPYAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLKGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 48 - protein, 429 aa, Homo sapiens
MFLKAVVLTLALVAVTGARAEVSADQVATVMWDYFSQLSSNAKEAVEHLQKSELTQQLNALFQDKLGEVNTYAGDLQKKLVPFATELHERLAKDSEKLKEEIRKELEEVRARLLPHANEVSQKIGENVRELQQRLEPYTDQLRTQVNTQTEQLRRQLTPYAQRMERVLRENADSLQTSLRPHADQLKAKIDQNVEELKERLTPYADEFKVKIDQTVEELRRSLAPYAQDAQEKLNHQLEGLAFQMKKNAEELKARISASAEELRQRLAPLAEDMRGNLRGNTEGLQKSLAELGGHLDRHVEEFRLRVEPYGENFNKALVQQMEQLRQKLGPHAGDVEGHLSFLEKDLRDKVNSFFSTFKEKESQDNTLSLPEPEQQREQQQEQQQEQEQEQQQQQEQQQQQEQQREQQQQEQQQEQQQEQVQMLAPLES

SEQ ID NO: 49 - protein, 395 aa, Homo sapiens
MFLKAAVLTLALVAITGTRAEVTSDQVANVVWDYFTQLSNNAKEAVEQFQKTDVTQQLSTLFQDKLGDASTYADGVHNKLVPFVVQLSGHLAKETERVKEEIKKELEDLRDRMMPHANKVTQTFGENMQKLQEHLKPYAVDLQDQINTQTQEMKLQLTPYIQRMQTTIKENVDNLHTSMMPLATNLKDKFNRNMEELKGHLTPRANELKATIDQNLEDLRRSLAPLTVGVQEKLNHQMEGLAFQMKKNAEELQTKVSAKIDQLQKNLAPLVEDVQSKVKGNTEGLQKSLEDLNRQLEQQVEEFRRTVEPMGEMFNKALVQQLEQFRQQLGPNSGEVESHLSFLEKSLREKVNSFMSTLEKKGSPDQPQALPLPEQAQEQAQEQAQEQVQPKPLES

SEQ ID NO: 50 - protein, 401 aa, Homo sapiens
GARAEVSADQVATVMWDYFSQLSSNAKEAVEHLQKSELTQQLNALFQDKLGEVNTYAGDLQKKLVPFATELHERLAKDSKKLKEEIRKELEEVRARLLPHANEVSQKIGENVRELQQRLEPYTDQLRTQVNTQTEQLRRQLTPYAQRMERVLRENADSLQTSLRPHADQLKAKIDQNVEELKGRLTPYADEFKVKIDQTVEELRRSLAPYAQDAQEKLNHQLEGLAFQMKKNAEELKARISASAEELRQRLAPLAEDMRGNLRGNTEGLQKSLAELGGHLDRHVEEFRLRVEPYGENFNKALVQQMEQLRQKLGPHAGDVEGHLSFLEKDLRDKVNSFFSTFKEKESQDNTLSLPEPEQQQEQQQEQEQQQEQQEEQQQQEQQQEQEQQQEQVQMLAPLES

SEQ ID NO: 51 - protein, 382 aa, Homo sapiens
MFLKAVVLSLALVAVTGARAEVNADQVATVMWDYFSQLGSNAKKAVEHLQKSELTQQLNTLFQDKLGEVNTYTEDLQKKLVPFATELHERLTKDSEKLKEEIRRELEELRARLLPHATEVSQKIGDNVRELQQRLGPFTGGLRTQVNTQVQQLQRQLKPYAERMESVLRQNIRNLEASVAPYADEFKAKIDQNVEELKGSLTPYAEELKAKIDQNVEELRRSLAPYAQDVQEKLNHQLEGLAFQMKKQAEELKAKISANADELRQKLVPVAENVHGHLKGNTEGLQKSLLELRSHLDQQVEEFRLKVEPYGETFNKALVQQVEDLRQKLGPLAGDVEGHLSFLEKDLRDKVNTFFSTLKEEASQGQSQALPAQEKAQAPLEG

SEQ ID NO: 52 - protein, 391 aa, Homo sapiens
MFLKAVVLTVALVAITGTQAEVTSDQVANVMWDYFTQLSNNAKEAVEQLQKTDVTQQLNTLFQDKLGNINTYADDLQNKLVPFAVQLSGHLTKETERVREEIQKELEDLRANMMPHANKVSQMFGDNVQKLQEHLRPYATDLQAQINAQTQDMKRQLTPYIQRMQTTIQDNVENLQSSMVPFANELKEKFNQNMEGLKGQLTPRANELKATIDQNLEDLRSRLAPLAEGVQEKLNHQMEGLAFQMKKNAEELQTKVSTNIDQLQKNLAPLVEDVQSKLKGNTEGLQKSLEDLNKQLDQQVEVFRRAVEPLGDKFNMALVQQMEKFRQQLGSDSGDVESHLSFLEKNLREKVSSFMSTLQKKGSPDQPLALPLPEQVQEQVQEQVQPKPLES

SEQ ID NO: 53 - protein, 51 aa, Homo sapiens
EPPTQKPKKIVNAKKDVVNTKMFEELKSRLDTLAQEVALLKEQQALQTVCL

SEQ ID NO: 54 - protein, 40 aa, Homo sapiens
IVNAKKDVVNTKMFEELKSRLDTLAQEVALLKEQQALQTV

SEQ ID NO: 55 - protein, 9 aa, Homo sapiens
CDLPQTHSL

SEQ ID NO: 56 - protein, 6 aa, Artificial Sequence
  Description of Artificial Sequence Synthetic 6xHis tag
HHHHHH

SEQ ID NO: 57 - protein, 310 aa, Artificial Sequence
  Description of Artificial Sequence Synthetic fusion polypeptide
MCDLPQTHSLGSHHHHHHGSVVAPPAPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 58 - DNA, 35 bp, Artificial Sequence
  Description of Artificial Sequence Synthetic primer N1
aaaaaagcggccgcgacaattcgcgcgcgaaggcg

SEQ ID NO: 59 - DNA, 36 bp, Artificial Sequence
  Description of Artificial Sequence Synthetic primer N2
aaaaaagcggccgctcactgcccgctttccagtcgg

SEQ ID NO: 60 - protein, 7 aa, Artificial Sequence
  Description of Artificial Sequence Synthetic IgA protease cleavage site peptide
VVAPPAP

SEQ ID NO: 61 - protein, 5 aa, Artificial Sequence
  Description of Artificial Sequence Synthetic IgA protease cleavage site peptide
PAPSP

SEQ ID NO: 62 - protein, 4 aa, Artificial Sequence
  Description of Artificial Sequence Synthetic IgA protease cleavage site peptide
PPSP

SEQ ID NO: 63 - protein, 4 aa, Artificial Sequence
  Description of Artificial Sequence Synthetic IgA protease cleavage site peptide
PPAP

SEQ ID NO: 64 - protein, 4 aa, Artificial Sequence
  Description of Artificial Sequence Synthetic IgA protease cleavage site peptide
PPTP

SEQ ID NO: 65 - protein, 4 aa, Artificial Sequence
  Description of Artificial Sequence Synthetic IgA protease cleavage site peptide
PPGP

SEQ ID NO: 66 - protein, 285 aa, Artificial Sequence
  Description of Artificial Sequence Synthetic Tetranectin-apolipoprotein A-I polypeptide
XPIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 67 - protein, 307 aa, Artificial Sequence
  Description of Artificial Sequence Synthetic Tetranectin-apolipoprotein A-I polypeptide with N-terminal His-tag
MHHHHHHXXXXXXXXXXXXXXXXXIVNAKKDVVNTKMFEELKSRLDTLAQEVALLKEQQALQTVDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 68 - protein, 4 aa, Artificial Sequence
  Description of Artificial Sequence Synthetic linker  peptide
GSAP

SEQ ID NO: 69 - protein, 4 aa, Artificial Sequence
  Description of Artificial Sequence Synthetic linker 2 peptide
GSGP

SEQ ID NO: 70 - protein, 4 aa, Artificial Sequence
  Description of Artificial Sequence Synthetic linker 3 peptide
GSSP

SEQ ID NO: 71 - protein, 4 aa, Artificial Sequence
  Description of Artificial Sequence Synthetic linker 4 peptide
GSPP

SEQ ID NO: 72 - protein, 4 aa, Artificial Sequence
  Description of Artificial Sequence Synthetic linker 5 peptide
GGGS

SEQ ID NO: 73 - protein, 5 aa, Artificial Sequence
  Description of Artificial Sequence Synthetic linker 6 peptide
GGGGS

SEQ ID NO: 74 - protein, 8 aa, Artificial Sequence
  Description of Artificial Sequence Synthetic linker 7 peptide
GGGSGGGS

SEQ ID NO: 75 - protein, 10 aa, Artificial Sequence
  Description of Artificial Sequence Synthetic linker 8 peptide
GGGGSGGGGS

SEQ ID NO: 76 - protein, 12 aa, Artificial Sequence
  Description of Artificial Sequence Synthetic linker 9 peptide
GGGSGGGSGGGS

SEQ ID NO: 77 - protein, 15 aa, Artificial Sequence
  Description of Artificial Sequence Synthetic linker 10 peptide
GGGGSGGGGSGGGGS

SEQ ID NO: 78 - protein, 6 aa, Artificial Sequence
  Description of Artificial Sequence Synthetic linker 11 peptide
GGGSAP

SEQ ID NO: 79 - protein, 6 aa, Artificial Sequence
  Description of Artificial Sequence Synthetic linker 12 peptide
GGGSGP

SEQ ID NO: 80 - protein, 6 aa, Artificial Sequence
  Description of Artificial Sequence Synthetic linker 13 peptide
GGGSSP

SEQ ID NO: 81 - protein, 6 aa, Artificial Sequence
  Description of Artificial Sequence Synthetic linker 14 peptide
GGGSPP

SEQ ID NO: 82 - protein, 7 aa, Artificial Sequence
  Description of Artificial Sequence Synthetic linker 15 peptide
GGGGSAP

SEQ ID NO: 83 - protein, 7 aa, Artificial Sequence
  Description of Artificial Sequence Synthetic linker 16 peptide
GGGGSGP

SEQ ID NO: 84 - protein, 7 aa, Artificial Sequence
  Description of Artificial Sequence Synthetic linker 17 peptide
GGGGSSP

SEQ ID NO: 85 - protein, 7 aa, Artificial Sequence
  Description of Artificial Sequence Synthetic linker 18 peptide
GGGGSPP

SEQ ID NO: 86 - protein, 10 aa, Artificial Sequence
  Description of Artificial Sequence Synthetic linker 19 peptide
GGGSGGGSAP

SEQ ID NO: 87 - protein, 10 aa, Artificial Sequence
  Description of Artificial Sequence Synthetic linker 20 peptide
GGGSGGGSGP

SEQ ID NO: 88 - protein, 10 aa, Artificial Sequence
  Description of Artificial Sequence Synthetic linker 21 peptide
GGGSGGGSSP

SEQ ID NO: 89 - protein, 10 aa, Artificial Sequence
  Description of Artificial Sequence Synthetic linker 22 peptide
GGGSGGGSPP

SEQ ID NO: 90 - protein, 14 aa, Artificial Sequence
  Description of Artificial Sequence Synthetic linker 23 peptide
GGGSGGGSGGGSAP

SEQ ID NO: 91 - protein, 14 aa, Artificial Sequence
  Description of Artificial Sequence Synthetic linker 24 peptide
GGGSGGGSGGGSGP

SEQ ID NO: 92 - protein, 14 aa, Artificial Sequence
  Description of Artificial Sequence Synthetic linker 25 peptide
GGGSGGGSGGGSSP

SEQ ID NO: 93 - protein, 14 aa, Artificial Sequence
  Description of Artificial Sequence Synthetic linker 26 peptide
GGGSGGGSGGGSPP

SEQ ID NO: 94 - protein, 7 aa, Artificial Sequence
  Description of Artificial Sequence Synthetic linker 27 peptide
GGGGSAP

SEQ ID NO: 95 - protein, 7 aa, Artificial Sequence
  Description of Artificial Sequence Synthetic linker 28 peptide
GGGGSGP

SEQ ID NO: 96 - protein, 7 aa, Artificial Sequence
  Description of Artificial Sequence Synthetic linker 29 peptide
GGGGSSP

SEQ ID NO: 97 - protein, 7 aa, Artificial Sequence
  Description of Artificial Sequence Synthetic linker 30 peptide
GGGGSPP

SEQ ID NO: 98 - protein, 12 aa, Artificial Sequence
  Description of Artificial Sequence Synthetic linker 31 peptide
GGGGSGGGGSAP

SEQ ID NO: 99 - protein, 12 aa, Artificial Sequence
  Description of Artificial Sequence Synthetic linker 32 peptide
GGGGSGGGGSGP

SEQ ID NO: 100 - protein, 12 aa, Artificial Sequence
  Description of Artificial Sequence Synthetic linker 33 peptide
GGGGSGGGGSSP

SEQ ID NO: 101 - protein, 12 aa, Artificial Sequence
  Description of Artificial Sequence Synthetic linker 34 peptide
GGGGSGGGGSPP

SEQ ID NO: 102 - protein, 17 aa, Artificial Sequence
  Description of Artificial Sequence Synthetic linker 35 peptide
GGGGSGGGGSGGGGSAP

SEQ ID NO: 103 - protein, 17 aa, Artificial Sequence
  Description of Artificial Sequence Synthetic linker 36 peptide
GGGGSGGGGSGGGGSGP

SEQ ID NO: 104 - protein, 17 aa, Artificial Sequence
  Description of Artificial Sequence Synthetic linker 37 peptide
GGGGSGGGGSGGGGSSP

SEQ ID NO: 105 - protein, 17 aa, Artificial Sequence
  Description of Artificial Sequence Synthetic linker 38 peptide
GGGGSGGGGSGGGGSPP